(19) United States
(12) Patent Application Publication
(10) Pub. No.: US 2024/0026447 A1
(43) Pub. Date: Jan. 25, 2024

(54) METHOD OF ASSESSING THE CIRCADIAN RHYTHM OF A SUBJECT AND/OR ASSESSING AND PREDICTING THE ATHLETIC PERFORMANCE OF SAID SUBJECT

(71) Applicant: MOREIRA BORRALHO RELÓGIO, Angela, Berlin (DE)

(72) Inventors: Janina HESSE, Berlin (DE); Alireza AKHONDZADEH BASTI, Berlin (DE)

(73) Assignee: Angela MOREIRA BORRALHO RELÓGIO, Berlin (DE)

(21) Appl. No.: 18/023,177

(22) PCT Filed: Aug. 27, 2021
(86) PCT No.: PCT/EP2021/073798

§ 371 Date: Feb. 24, 2023

(30) Foreign Application Priority Data
Aug. 27, 2020 (EP) 20193227.4

(51) Int. Cl. C12Q 1/6876; C12Q 1/6851; G16B 25/10; G16B 40/20

(52) U.S. Cl. CPC C12Q 1/6876; C12Q 1/6851; G16B 25/10; G16B 40/20; C12Q 2600/158; C12Q 2600/124

(57) ABSTRACT

A method of assessing the circadian rhythm of a subject and/or assessing and predicting the athletic performance of said subject, including the steps of providing at least three samples of saliva, more preferably four samples of saliva, from the subject, wherein the samples have been taken at different time points over the day and determining gene expression of at least two members of genes for the core-clock network, in particular of at least two members of the group including ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, in particular of BMAL1 and PER2, in each sample. And the step of assessing and predicting by a computational step based on the expression levels of BMAL1 and PER2 over the day the circadian rhythm of the subject and/or the individual diurnal athletic performance times.

Subject matter of the present invention is a method of assessing the circadian rhythm of a subject and/or assessing and predicting the athletic performance of said subject, wherein said method comprises the steps of:
Providing at least three samples of saliva, more preferably four samples of saliva, from said subject, wherein said samples have been taken at different time points over the day,
Determining gene expression of at least two members of genes for the core-clock network, in particular of at least two members of the group comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, in each of said samples, and
Assessing and predicting by means of a computational step based on said expression levels of ARNTL (BMAL1) and PER2 over the day the circadian rhythm of said subject and/or the individual diurnal athletic performance times.

### STATE OF THE ART

The biological clock (also known as circadian clock) regulates several aspects of physiology and behavior via cellular and molecular mechanisms and plays a vital role in maintaining proper human health. This is no wonder, since about half of all human genes are rhythmically expressed in at least one tissue. The disruption of circadian rhythms is associated to several diseases including sleep disorders, depression, diabetes, Alzheimer's disease, obesity and cancer. This disruption might result from conflicting external (environmental) or internal (feeding/resting) signals that are not in synchrony with the internal biological time. This not only affects shift workers, but also most people subjected to societal routine (social jet lag). Furthermore, a desynchronized circadian clock was shown to negatively affect an individual's wellbeing, in particular in the context of metabolism, as well as physical and mental (cognitive) performance.

Recent studies have reported a role for clock dysregulation in the pathologies mentioned above, raising increased awareness from scientists, clinicians and the public. Thus, it is crucial to characterize the individual's internal clock and to adjust the external/internal factors, to avoid or to overcome circadian rhythm disruption. Moreover, the time for certain activities, such as sleep, sports or medicine intake, can be optimized based on the individual's internal timing. A proper functioning circadian clock, synchronized with the individual's behavioral habits, will improve fitness and reduce therapy/recovery time in patients.

Yet, the available methods for clock assessment are either not very accurate or mostly invasive and often require medical assistance over a period of several hours (tedious, time-consuming, and cost-intensive). So far, there are the following methods among the molecular approaches to determine the biological clock in humans:
1. Determination of time-point at which endogenous melatonin (or cortisol), reaches a predefined threshold value concentration (dim-light-melatonin-onset, aka DLMO). To do this, several blood or saliva samples (usually every 0.5-1 hour) under dim-light conditions are taken to determine the internal clock.
2. Blood samples (with one or more sampling times) are also used to identify biomarkers using a machine learning approach that could determine the expression phase of “time-indicating” genes and thus the internal clock timing.
3. In addition, the circadian phase (peak time of secretion/expression) in hair samples (hair follicle cells) or urine samples could reflect that of the individual behavioral rhythm. This strategy is more suitable for assessing the human peripheral clock.

As mentioned above, current methods of clock assessment are either invasive, require medical supervision, are time-consuming or do not provide detailed information on the gene expression level. Saliva plays numerous protective roles for oral tissue maintenance in humans. Adequate salivary flow and saliva content are directly related to health status. Previous studies have shown the potential of saliva and salivary transcriptome as a diagnostic tool, which underlines the importance of saliva sampling as a non-invasive diagnostic method. Time-course saliva sampling is also commonly used for estimating the evening dim-light melatonin onset (DLMO) in humans in order to determine their circadian phase (peak time of secretion/expression); a method that requires controlled dim-light conditions during the entire sampling time of 5-6 h.

The circadian rhythm was previously modeled with different approaches, starting with models that simply show oscillations such as phase-oscillators, and going up to molecular models, which model (part of) the molecular interactions underlying the circadian clock. In the present disclosure it is focused on molecular models, because these contain biological information that might be useful for predictions. Molecular models with simple feedback loops are often based on Goodwin's oscillator, e.g. (Ruoff and Rensing 1996), but the level of detail may also be extensive (Forger and Peskin 2003).

An objective is to provide a model at an intermediate state of complexity, complex enough to capture a significant part of the genetic network, but if the model is too complex, we cannot fit our data to the model without significant overfitting. Relogio et al. have published a model at this level of complexity, with 19 dynamical variables, which we use in the following (Relógio et al. 2011).

Other studies with mammalian clock models are named below, including respective literal citations from the respective papers.

(Becker-Weimann et al. 2004): “We present a mathematical model that reflects the essential features of the mammalian circadian oscillator to characterize the differential roles of negative and positive feedback loops. The oscillations that are obtained have a 24-h period and are robust toward parameter variations even when the positive feedback is replaced by a constantly expressed activator. This demonstrates the crucial role of the negative feedback for rhythm generation.” [7 dynamical variables]

(Forger and Peskin 2003): “Here we develop a detailed distinctly mammalian model by using mass action kinetics. Parameters for our model are found from experimental data by using a coordinate search method. The model accurately predicts the phase of entrainment, amplitude of oscillation, and shape of time profiles of clock mRNAs and proteins and is also robust to parameter changes and mutations.” [74 dynamical variables] There also is a stochastic version of this model (Forger and Peskin 2005).

(Leloup and Goldbeter 2003): “We present a computational model for the mammalian circadian clock based on the intertwined positive and negative regulatory loops involving the Per, Cry, Arntl (Bmal1), Clock, and Rev-Erb a genes. In agreement with experimental observations, the model can give rise to sustained circadian oscillations in continuous darkness, characterized by an antiphase relationship between Per/Cry/Rev-Erba and Arntl (Bmal1) mRNAs. Sustained oscillations correspond to the rhythms autonomously generated by suprachiasmatic nuclei. For other parameter values, damped oscillations can also be obtained in the model. These oscillations, which transform into sustained oscillations when coupled to a periodic signal, correspond to rhythms produced by peripheral tissues.” [19 dynamical variables] Bifurcation analysis of this model published in (Leloup and Goldbeter 2004).

(Mirsky et al. 2009): “In this study, we built a mathematical model from the regulatory structure of the intracellular circadian clock in mice and identified its parameters using an iterative evolutionary strategy, with minimum cost achieved through conformance to phase separations seen in cell-autonomous oscillators. The model was evaluated against the experimentally observed cell-autonomous circadian phenotypes of gene knockouts, particularly retention of rhythmicity and changes in expression level of molecular clock components.” “Most importantly, our model addresses the overlapping but differential functions of CRY1 and CRY2 in the clock mechanism: They antagonistically regulate period length and differentially control rhythm persistence and amplitude” [21 dynamical variables] This model focuses on the phase relationship between different genes.

(Kim and Forger 2012): “To understand the biochemical mechanisms of this timekeeping, we have developed a detailed mathematical model of the mammalian circadian clock. Our model can accurately predict diverse experimental data including the phenotypes of mutations or knockdown of clock genes as well as the time courses and relative expression of clock transcripts and proteins. Using this model, we show how a universal motif of circadian timekeeping, where repressors tightly bind activators rather than directly binding to DNA, can generate oscillations when activators and repressors are in stoichiometric balance.” [Ref: Kim, Jae Kyoung, and Daniel B Forger. 2012. “A Mechanism for Robust Circadian Timekeeping via Stoichiometric Balance.” Molecular Systems Biology 8 (December): 630. https://doi.org/10.1038/msb.2012.62.]

(Jolley et al. 2014): Focus on a new mechanism via D-box, and modern parameter estimation.

Besides these models of the mammalian circadian clock, various models have been published for non-mammalian systems, and intensively investigated, e.g. by bifurcation analysis.

### SUMMARY OF THE INVENTION

The role of the circadian clock in the daily fluctuations of sports performance in healthy individuals has been explored. Molecular (gene expression) and physiological (biomechanical muscle properties) features in humans have been measured, and the athletic performance of healthy individuals at different times of the day was recorded based on strength and endurance tests. The data shows circadian variations in gene expression, sports performance and muscle properties, and a correlation between the sports performance and the molecular and physiological data has been found. Computational/machine learning approaches using accessible human biological material in time series studies have been applied.

Establishing human saliva as the biological source of material, core-clock gene expression (e.g. ARNTL (BMAL1) and PER2) has been analyzed over time and compared to biomechanical muscle properties and athletic performance. In particular, it has been shown that e.g. ARNTL (BMAL1) and PER2 expression display distinctive daily fluctuations in saliva samples, which correlate to the oscillation amplitude and peak time of athletic performance during the day.

In addition to the core-clock genes the expression of the clock- and sports-related gene AKT1, a serine-threonine protein kinase, involved in metabolism and the response to aerobic exercise has been measured and it was shown that fluctuations of AKT1 expression during the day for all participants tested have significant correlation to the temporal profile of ARNTL (BMAL1), which hints towards the circadian regulation of exercise and athletic performance throughout the day.

According to the present invention personalized predictions of athletic performance have been enabled. The inventors' study revealed that the variation in the expression of the core-clock genes ARNTL (BMAL1) and PER2 across the day, their ratio of expression (e.g. ARNTL (BMAL1) over PER2), and their average expression can be used as predictors for individual optimal sports performance time, both for strength exercises and endurance exercises.

In one embodiment, with regard to PER2 the peak time of expression is used for the computational steps. In one embodiment, with regard to ARNTL (BMAL1) the overall difference in expression levels (between participants) is used for the computational steps.

### DETAILED DESCRIPTION OF THE INVENTION

Subject matter of the present invention is a method of assessing the circadian rhythm of a subject and/or assessing and predicting the athletic performance of said subject, wherein said method comprises the steps of:
Providing at least three samples of saliva, more preferably four samples of saliva, from said subject, wherein said samples have been taken at different time points over the day,
Determining gene expression of at least two members of genes for the core-clock network, in particular of at least two members of the group comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, in particular of ARNTL (BMAL1) and PER2, in each of said samples, and
Assessing and predicting by means of a computational step based on said expression levels of at least two members of the groups comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, over the day the circadian rhythm of said subject and/or the individual diurnal athletic performance times.

Subject matter of the present invention is a method of assessing the circadian rhythm of a subject and/or assessing and predicting the athletic performance of said subject, wherein said method comprises the steps of:
Providing at least three samples of saliva, more preferably four samples of saliva, from said subject, wherein said samples have been taken at different time points over the day,
Determining gene expression of ARNTL (BMAL1) and PER2 in each of said samples, and
Assessing and predicting by means of a computational step based on said expression levels of ARNTL (BMAL1) and PER2 over the day the circadian rhythm of said subject and/or the individual diurnal athletic performance times.

In one embodiment of the invention gene expression is determined using a method selected from quantitative PCR (RT-qPCR), NanoString, sequencing and microarray. Any other method for determining gene expression may be used.

In one embodiment of the invention gene expression is determined using quantitative PCR (RT-qPCR).

In one embodiment of the invention gene expression is determined using NanoString, see e.g. Geiss G, et al., Direct multiplexed measurement of gene expression with color-coded probe pairs, 26: 317-25 (2008), Nature Biotechnology, Feb. 8, 2008.

BMAL1 is also known as ARNTL, Aryl hydrocarbon receptor nuclear translocator-like protein 1 (ARNTL) Or Brain and Muscle ARNT-Like 1 (BMAL1)

The sequence of cDNA ARNTL (BMAL1) comprises
SEQ ID No. 1:
>ENST00000389707.8 ARNTL-201 cdna:
protein_coding
ATGGCAGACCAGAGAATGGACATTTCTTCAACCATCAGTGATTTC
ATGTCCCCGGGCCCCACCGACCTGCTTTCCAGCTCTCTTGGTACC
AGTGGTGTGGATTGCAACCGCAAACGGAAAGGCAGCTCCACTGAC
TACCAAGAAAGCATGGACACAGACAAAGATGACCCTCATGGAAGG
TTAGAATATACAGAACACCAAGGAAGGATAAAAAATGCAAGGGAA
GCTCACAGTCAGATTGAAAAGCGGCGTCGGGATAAAATGAACAGT
TTTATAGATGAATTGGCTTCTTTGGTACCAACATGCAACGCAATG
TCCAGGAAATTAGATAAACTTACTGTGCTAAGGATGGCTGTTCAG
CACATGAAAACATTAAGAGGTGCCACCAATCCATACACAGAAGCA
AACTACAAACCAACTTTTCTATCAGACGATGAATTGAAACACCTC
ATTCTCAGGGCAGCAGATGGATTTTTGTTTGTCGTAGGATGTGAC
CGAGGGAAGATACTCTTTGTCTCAGAGTCTGTCTTCAAGATCCTC
AACTACAGCCAGAATGATCTGATTGGTCAGAGTTTGTTTGACTAC
CTGCATCCTAAAGATATTGCCAAAGTCAAGGAGCAGCTCTCCTCC
TCTGACACCGCACCCCGGGAGCGGCTCATAGATGCAAAAACTGGA
CTTCCAGTTAAAACAGATATAACCCCTGGGCCATCTCGATTATGT
TCTGGAGCACGACGTTCTTTCTTCTGTAGGATGAAGTGTAACAGG
CCTTCAGTAAAGGTTGAAGACAAGGACTTCCCCTCTACCTGCTCA
AAGAAAAAAGATCGAAAAAGCTTCTGCACAATCCACAGCACAGGC
TATTTGAAAAGCTGGCCACCCACAAAGATGGGGCTGGATGAAGAC
AACGAACCAGACAATGAGGGGTGTAACCTCAGCTGCCTCGTCGCA
ATTGGACGACTGCATTCTCATGTAGTTCCACAACCAGTGAACGGG
GAAATCAGGGTGAAATCTATGGAATATGTTTCTCGGCACGCGATA
GATGGAAAGTTTGTTTTTGTAGACCAGAGGGCAACAGCTATTTTG
GCATATTTACCACAAGAACTTCTAGGCACATCGTGTTATGAATAT
TTTCACCAAGATGACATAGGACATCTTGCAGAATGTCATAGGCAA
GTTTTACAGACGAGAGAAAAAATTACAACTAATTGCTATAAATTT
AAAATCAAAGATGGTTCTTTTATCACACTACGGAGTCGATGGTTC
AGTTTCATGAACCCTTGGACCAAGGAAGTAGAATATATTGTCTCA
ACTAACACTGTTGTTTTAGCCAACGTCCTGGAAGGCGGGGACCCA
ACCTTCCCACAGCTCACAGCATCCCCCCACAGCATGGACAGCATG
CTGCCCTCTGGAGAAGGTGGCCCAAAGAGGACCCACCCCACTGTT
CCAGGGATTCCAGGGGGAACCCGGGCTGGGGCAGGAAAAATAGGC
CGAATGATTGCTGAGGAAATCATGGAAATCCACAGGATAAGAGGG
TCATCGCCTTCTAGCTGTGGCTCCAGCCCATTGAACATCACGAGT
ACGCCTCCCCCTGATGCCTCTTCTCCAGGAGGCAAGAAGATTTTA
AATGGAGGGACTCCAGACATTCCTTCCAGTGGCCTACTATCAGGC
CAGGCTCAGGAGAACCCAGGTTATCCATATTCTGATAGTTCTTCT
ATTCTTGGTGAGAACCCCCACATAGGTATAGACATGATTGACAAC
GACCAAGGATCAAGTAGTCCCAGTAATGATGAGGCAGCAATGGCT
GTCATCATGAGCCTCTTGGAAGCAGATGCTGGACTGGGTGGCCCT
GTTGACTTTAGTGACTTGCCATGGCCGCTGTAA
The sequence of cDNA ARNTL2 comprises
SEQ ID No. 2
>ENST00000266503.9 ARNTL2-202 cdna:
protein_coding
ATGGCGGCGGAAGAGGAGGCTGCGGCGGGAGGTAAAGTGTTGAGA
GAGGAGAACCAGTGCATTGCTCCTGTGGTTTCCAGCCGCGTGAGT
CCAGGGACAAGACCAACAGCTATGGGGTCTTTCAGCTCACACATG
ACAGAGTTTCCACGAAAACGCAAAGGAAGTGATTCAGACCCATCC
CAGTCAGGAATCATGACAGAAAAAGTGGTGGAAAAGCTTTCTCAG
AATCCCCTTACCTATCTTCTTTCAACAAGGATAGAAATATCAGCC
TCCAGTGGCAGCAGAGTGGAAGATGGTGAACACCAAGTTAAAATG
AAGGCCTTCAGAGAAGCTCATAGCCAAACTGAAAAGCGGAGGAGA
GATAAAATGAATAACCTGATTGAAGAACTGTCTGCAATGATCCCT
CAGTGCAACCCCATGGCGCGTAAACTGGACAAACTTACAGTTTTA
AGAATGGCTGTTCAACACTTGAGATCTTTAAAAGGCTTGACAAAT
TCTTATGTGGGAAGTAATTATAGACCATCATTTCTTCAGGATAAT
GAGCTCAGACATTTAATCCTTAAGACTGCAGAAGGCTTCTTATTT
GTGGTTGGATGTGAAAGAGGAAAAATTCTCTTCGTTTCTAAGTCA
GTCTCCAAAATACTTAATTATGATCAGGCTAGTTTGACTGGACAA
AGCTTATTTGACTTCTTACATCCAAAAGATGTTGCCAAAGTAAAG
GAACAACTTTCTTCTTTTGATATTTCACCAAGAGAAAAGCTAATA
GATGCCAAAACTGGTTTGCAAGTTCACAGTAATCTCCACGCTGGA
AGGACACGTGTGTATTCTGGCTCAAGACGATCTTTTTTCTGTCGG
ATAAAGAGTTGTAAAATCTCTGTCAAAGAAGAGCATGGATGCTTA
CCCAACTCAAAGAAGAAAGAGCACAGAAAATTCTATACTATCCAT
TGCACTGGTTACTTGAGAAGCTGGCCTCCAAATATTGTTGGAATG
GAAGAAGAAAGGAACAGTAAGAAAGACAACAGTAATTTTACCTGC
CTTGTGGCCATTGGAAGATTACAGCCATATATTGTTCCACAGAAC
AGTGGAGAGATTAATGTGAAACCAACTGAATTTATAACCCGGTTT
GCAGTGAATGGAAAATTTGTCTATGTAGATCAAAGGGCAACAGCG
ATTTTAGGATATCTGCCTCAGGAACTTTTGGGAACTTCTTGTTAT
GAATATTTTCATCAAGATGACCACAATAATTTGACTGACAAGCAC
AAAGCAGTTCTACAGAGTAAGGAGAAAATACTTACAGATTCCTAC
AAATTCAGAGCAAAAGATGGCTCTTTTGTAACTTTAAAAAGCCAA
TGGTTTAGTTTCACAAATCCTTGGACAAAAGAACTGGAATATATT
GTATCTGTCAACACTTTAGTTTTGGGACATAGTGAGCCTGGAGAA
GCATCATTTTTACCTTGTAGCTCTCAATCATCAGAAGAATCCTCT
AGACAGTCCTGTATGAGTGTACCTGGAATGTCTACTGGAACAGTA
CTTGGTGCTGGTAGTATTGGAACAGATATTGCAAATGAAATTCTG
GATTTACAGAGGTTACAGTCTTCTTCATACCTTGATGATTCGAGT
CCAACAGGTTTAATGAAAGATACTCATACTGTAAACTGCAGGAGT
ATGTCAAATAAGGAGTTGTTTCCACCAAGTCCTTCTGAAATGGGG
GAGCTAGAGGCTACCAGGCAAAACCAGAGTACTGTTGCTGTCCAC
AGCCATGAGCCACTCCTCAGTGATGGTGCACAGTTGGATTTCGAT
GCCCTATGTGACAATGATGACACAGCCATGGCTGCATTTATGAAT
TACTTAGAAGCAGAGGGGGGCCTGGGAGACCCTGGGGACTTCAGT
GACATCCAGTGGACCCTCTAG
The sequence of cDNA PER1 comprises
SEQ ID No. 3
>ENST00000317276.9 PER1-201 cdna:
protein_coding
ATGAGTGGCCCCCTAGAAGGGGCTGATGGGGGAGGGGACCCCAGG
CCTGGGGAATCATTTGTCCTGGGGGCGTCCCATCCCCTGGGCCCC
CACAGCACCGGCCTTGCCCAGGCCCCAGCCTGGCCGATGACACCG
ATGCCAACAGCAATGGTTCAAGTGGCAATGAGTCCAACGGGCATG
AGTCTAGAGGCGCATCTCAGCGGAGCTCACACAGCTCCTCCTCAG
GCAACGGCAAGGACTCAGCCCTGCTGGAGACCACTGAGAGCAGCA
AGAGCACAAACTCTCAGAGCCCATCCCCACCCAGCAGTTCCATTG
CCTACAGCCTCCTGAGTGCCAGCTCAGAGCAGGACAACCCGTCCA
CCAGTGGCTGCAGCAGTGAACAGTCAGCCCGGGCAAGGACTCAGA
AGGAACTCATGACAGCACTTCGAGAGCTCAAGCTTCGACTGCCGC
CAGAGCGCCGGGGCAAGGGCCGCTCTGGGACCCTGGCCACGCTGC
AGTACGCACTGGCCTGTGTCAAGCAGGTGCAGGCCAACCAGGAAT
ACTACCAGCAGTGGAGCCTGGAGGAGGGCGAGCCTTGCTCCATGG
ACATGTCCACCTATACCCTGGAGGAGCTGGAGCACATCACGTCTG
AGTACACACTTCAGAACCAGGATACCTTCTCAGTGGCTGTCTCCT
TCCTGACGGGCCGAATCGTCTACATTTCGGAGCAGGCAGCCGTCC
TGCTGCGTTGCAAGCGGGACGTGTTCCGGGGTACCCGCTTCTCTG
AGCTCCTGGCTCCCCAGGATGTGGGAGTCTTCTATGGTTCCACTG
CTCCATCTCGCCTGCCCACCTGGGGCACAGGGGCCTCAGCAGGTT
CAGGCCTCAGGGACTTTACCCAGGAGAAGTCCGTCTTCTGCCGTA
TCAGAGGAGGTCCTGACCGGGATCCAGGGCCTCGGTACCAGCCAT
TCCGCCTAACCCCGTATGTGACCAAGATCCGGGTCTCAGATGGGG
CCCCTGCACAGCCGTGCTGCCTGCTGATTGCAGAGCGCATCCATT
CGGGTTACGAAGCTCCCCGGATACCCCCTGACAAGAGGATTTTCA
CTACGCGGCACACACCCAGCTGCCTCTTCCAGGATGTGGATGAAA
GGGCTGCCCCCCTGCTGGGCTACCTGCCCCAGGACCTCCTGGGGG
CCCCAGTGCTCCTGTTCCTGCATCCTGAGGACCGACCCCTCATGC
TGGCTATCCACAAGAAGATTCTGCAGTTGGCGGGCCAGCCCTTTG
ACCACTCCCCTATCCGCTTCTGTGCCCGCAACGGGGAGTATGTCA
CCATGGACACCAGCTGGGCTGGCTTTGTGCACCCCTGGAGCCGCA
AGGTAGCCTTCGTGTTGGGCCGCCACAAAGTACGCACGGCCCCCC
TGAATGAGGACGTGTTCACTCCCCCGGCCCCCAGCCCAGCTCCCT
CCCTGGACACTGATATCCAGGAGCTGTCAGAGCAGATCCACCGGC
TGCTGCTGCAGCCCGTCCACAGCCCCAGCCCCACGGGACTCTGTG
GAGTCGGCGCCGTGACATCCCCAGGCCCTCTCCACAGCCCTGGGT
CCTCCAGTGATAGCAACGGGGGTGATGCAGAGGGGCCTGGGCCTC
CTGCGCCAGTGACTTTCCAGCAGATCTGTAAGGATGTGCATCTGG
TGAAGCACCAGGGCCAGCAGCTTTTTATTGAGTCTCGGGCCCGGC
CTCAGTCCCGGCCCCGCCTCCCTGCTACAGGCACGTTCAAGGCCA
AGGCCCTTCCCTGCCAATCCCCAGACCCAGAGCTGGAGGCGGGTT
CTGCTCCCGTCCAGGCCCCACTAGCCTTGGTCCCTGAGGAGGCCG
AGAGGAAAGAAGCCTCCAGCTGCTCCTACCAGCAGATCAACTGCC
TGGACAGCATCCTCAGGTACCTGGAGAGCTGCAACCTCCCCAGCA
CCACTAAGCGTAAATGTGCCTCCTCCTCCTCCTATACCACCTCCT
CAGCCTCTGACGACGACAGGCAGAGGACAGGTCCAGTCTCTGTGG
GGACCAAGAAAGATCCGCCGTCAGCAGCGCTGTCTGGGGAGGGGG
CCACCCCACGGAAGGAGCCAGTGGTGGGAGGCACCCTGAGCCCGC
TCGCCCTGGCCAATAAGGCGGAGAGTGTGGTGTCCGTCACCAGTC
AGTGTAGCTTCAGCTCCACCATCGTCCATGTGGGAGACAAGAAGC
CCCCGGAGTCGGACATCATCATGATGGAGGACCTGCCTGGCCTAG
CCCCAGGCCCAGCCCCCAGCCCAGCCCCCAGCCCCACAGTAGCCC
CTGACCCAGCCCCAGACGCCTACCGTCCAGTGGGGCTGACCAAGG
CCGTGCTGTCCCTGCACACACAGAAGGAAGAGCAAGCCTTCCTCA
GCCGCTTCCGAGACCTGGGCAGGCTGCGTGGACTCGACAGCTCTT
CCACAGCTCCCTCAGCCCTTGGCGAGCGAGGCTGCCACCACGGCC
CCGCACCCCCAAGCCGCCGACACCACTGCCGATCCAAAGCCAAGC
GCTCACGCCACCACCAGAACCCTCGGGCTGAAGCGCCCTGCTATG
TCTCACACCCCTCACCCGTGCCACCCTCCACCCCCTGGCCCACCC
CACCAGCCACTACCCCCTTCCCAGCGGTTGTCCAGCCCTACCCTC
TCCCAGTGTTCTCTCCTCGAGGAGGCCCCCAGCCTCTTCCCCCTG
CTCCCACATCTGTGCCCCCAGCTGCTTTCCCCGCCCCTTTGGTGA
CCCCAATGGTGGCCTTGGTGCTCCCTAACTATCTGTTCCCAACCC
CATCCAGCTATCCTTATGGGGCACTCCAGACCCCTGCTGAAGGGC
CTCCCACTCCTGCCTCGCACTCCCCTTCTCCATCCTTGCCCGCCC
TCGCCCCGAGTCCTCCTCACCGCCCGGACTCTCCACTGTTCAACT
CGAGATGCAGCTCTCCACTCCAGCTCAATCTGCTGCAGCTGGAGG
AGCTCCCCCGTGCTGAGGGGGCTGCTGTTGCAGGAGGCCCTGGGA
GCAGTGCCGGGCCCCCACCTCCCAGTGCGGAGGCTGCTGAGCCAG
AGGCCAGACTGGCGGAGGTCACTGAGTCCTCCAATCAGGACGCAC
TTTCCGGCTCCAGTGACCTGCTCGAACTTCTGCTGCAAGAGGACT
CGCGCTCCGGCACAGGCTCCGCAGCCTCGGGCTCCTTGGGCTCTG
GCTTGGGCTCTGGGTCTGGTTCAGGCTCCCATGAAGGGGGCAGCA
CCTCAGCCAGCATCACTCGCAGCAGCCAGAGCAGCCACACAAGCA
AATACTTTGGCAGCATCGACTCTTCCGAGGCTGAGGCTGGGGCTG
CTCGGGGCGGGGCTGAGCCTGGGGACCAGGTGATTAAGTACGTGC
TCCAGGATCCCATTTGGCTGCTCATGGCCAATGCTGACCAGCGCG
TCATGATGACCTACCAGGTGCCCTCCAGGGACATGACCTCTGTGC
TGAAGCAGGATCGGGAGCGGCTCCGAGCCATGCAGAAGCAGCAGC
CTCGGTTTTCTGAGGACCAGCGGCGGGAACTGGGTGCTGTGCACT
CCTGGGTCCGGAAGGGCCAACTGCCTCGGGCTCTTGATGTGATGG
CCTGTGTGGACTGTGGGAGCAGCACCCAAGATCCTGGTCACCCTG
ATGACCCACTCTTCTCAGAGCTGGATGGACTGGGGCTGGAGCCCA
TGGAAGAGGGTGGAGGCGAGCAGGGCAGCAGCGGTGGCGGCAGTG
GTGAGGGAGAGGGCTGCGAGGAGGCCCAAGGCGGGGCCAAGGCTT
CAAGCTCTCAGGACTTGGCTATGGAGGAGGAGGAAGAAGGCAGGA
GCTCATCCAGTCCAGCCTTACCTACAGCAGGAAACTGCACCAGCT
AG
The sequence of PER2 cDNA comprises
SEQ ID No. 4:
>ENST00000254657.8 PER2-201 cdna:
protein_coding
ATGAATGGATACGCGGAATTTCCGCCCAGCCCCAGTAACCCCACC
AAGGAGCCCGTGGAGCCCCAGCCCAGCCAGGTCCCACTGCAGGAA
GATGTGGACATGAGCAGTGGCTCCAGTGGACATGAGACCAACGAA
AACTGCTCCACGGGGGGGGACTCGCAGGGCAGTGACTGTGACGAC
AGTGGGAAGGAGCTGGGGATGCTGGTGGAGCCACCGGATGCCCGC
CAGAGTCCAGATACCTTTAGCCTGATGATGGCAAAATCTGAACAC
AACCCATCTACAAGTGGCTGCAGTAGCGACCAGTCTTCGAAAGTG
GACACACACAAAGAACTGATAAAAACACTAAAGGAGCTGAAGGTC
CACCTCCCTGCAGACAAGAAGGCCAAGGGCAAGGCCAGTACGCTG
GCCACCTTGAAGTACGCCCTCAGGAGCGTGAAGCAGGTGAAAGCC
AATGAAGAGTATTACCAGCTGCTGATGTCCAGCGAGGGTCACCCC
TGTGGAGCAGACGTGCCCTCCTACACCGTGGAGGAGATGGAGAGC
GTTACCTCTGAGCACATTGTGAAGAATGCCGATATGTTTGCGGTG
GCCGTGTCCCTGGTGTCTGGGAAGATCCTGTACATCTCTGACCAG
GTTGCATCCATATTTCACTGTAAAAGAGATGCCTTCAGCGATGCC
AAGTTTGTGGAGTTCCTGGCGCCTCACGATGTGGGCGTGTTCCAC
AGTTTCACCTCCCCGTACAAGCTTCCCTTGTGGAGCATGTGCAGT
GGAGCAGATTCTTTTACTCAAGAATGCATGGAGGAGAAATCTTTC
TTTTGCCGTGTCAGTGTCCGGAAAAGCCACGAGAATGAAATCCGC
TACCACCCCTTCCGCATGACGCCCTACCTGGTCAAGGTGCGGGAC
CAACAAGGTGCTGAGAGTCAGCTTTGCTGCCTTCTGCTGGCAGAG
AGAGTGCACTCTGGTTATGAAGCCCCTAGAATTCCTCCTGAAAAG
AGAATTTTTACAACCACCCATACACCAAATTGTTTGTTCCAGGAT
GTGGATGAAAGGGCGGTCCCTCTCCTGGGCTACCTACCTCAGGAC
CTGATTGAAACCCCAGTGCTCGTGCAGCTCCACCCTAGTGACAGG
CCCTTGATGCTGGCCATCCACAAAAAGATCCTGCAGTCAGGCGGG
CAGCCTTTCGACTATTCTCCCATTCGGTTTCGCGCCCGGAACGGA
GAGTACATCACGTTGGACACCAGCTGGTCCAGCTTCATCAACCCA
TGGAGCAGGAAAATCTCCTTCATCATTGGGAGGCACAAAGTCAGG
GTGGGCCCTTTGAATGAGGACGTGTTTGCAGCCCACCCCTGCACA
GAGGAGAAGGCCCTGCACCCCAGCATTCAGGAGCTCACAGAGCAG
ATCCACCGGCTCCTGCTGCAGCCCGTCCCCCACAGCGGCTCCAGT
GGCTACGGGAGTCTGGGCAGCAACGGGTCCCACGAGCACCTTATG
AGCCAGACCTCCTCCAGCGACAGCAACGGCCATGAGGACTCACGC
CGGAGGAGAGCCGAAATTTGTAAAAATGGTAACAAGACCAAAAAT
AGAAGTCATTATTCTCATGAATCTGGAGAACAAAAGAAAAAATCC
GTTACAGAAATGCAAACTAATCCCCCAGCTGAGAAGAAAGCTGTC
CCTGCCATGGAAAAGGACAGCCTGGGGGTCAGCTTCCCCGAGGAG
TTGGCCTGCAAGAACCAGCCCACCTGCTCCTACCAGCAGATCAGC
TGCTTGGACAGCGTCATCAGGTACTTGGAGAGCTGCAATGAGGCT
GCCACCCTGAAGAGGAAATGCGAGTTCCCAGCAAACGTCCCAGCG
CTAAGGTCCAGTGATAAGCGGAAGGCCACAGTCAGCCCAGGGCCA
CACGCTGGAGAGGCAGAGCCGCCCTCCAGGGTGAACAGCCGCACG
GGAGTAGGTACGCACCTGACCTCGCTGGCACTGCCGGGCAAGGCA
GAGAGTGTGGCGTCGCTCACCAGCCAGTGCAGCTACAGCAGCACC
ATCGTCCATGTGGGAGACAAGAAGCCGCAGCCGGAGTTAGAGATG
GTGGAAGATGCTGCGAGTGGGCCAGAATCCCTGGACTGCCTGGCG
GGCCCTGCCCTGGCCTGTGGTCTCAGCCAAGAGAAGGAGCCCTTC
AAGAAGCTGGGCCTCACCAAGGAGGTACTCGCTGCACACACACAG
AAGGAGGAGCAGAGCTTCCTGCAGAAGTTCAAAGAAATAAGAAAA
CTCAGCATTTTCCAGTCCCACTGCCATTACTACTTGCAAGAAAGA
TCCAAGGGGCAGCCAAGTGAACGAACTGCCCCTGGACTAAGAAAT
ACTTCCGGAATAGATTCACCTTGGAAAAAAACAGGAAAGAACAGA
AAATTGAAGTCCAAGCGGGTCAAACCTCGAGACTCATCTGAGAGC
ACCGGATCTGGGGGGCCCGTGTCCGCCCGGCCCCCGCTGGTGGGC
TTGAACGCCACAGCCTGGTCACCCTCAGACACGTCCCAGTCCAGC
TGCCCAGCCGTGCCCTTTCCCGCCCCAGTGCCAGCAGCTTATTCA
CTGCCCGTGTTTCCAGCGCCAGGGACTGTGGCAGCACCCCCGGCA
CCTCCCCACGCCAGCTTCACAGTGCCTGCTGTGCCCGTGGACCTC
CAGCACCAGTTTGCAGTCCAGCCCCCACCTTTCCCTGCCCCTTTG
GCGCCTGTCATGGCATTCATGCTACCCAGTTATTCCTTCCCCTCG
GGGACCCCAAACCTGCCCCAGGCCTTCTTCCCCAGCCAGCCTCAG
TTTCCGAGCCACCCCACACTCACATCCGAGATGGCCTCTGCCTCA
CAGCCTGAGTTCCCCAGCCGGACCTCGATCCCCAGACAGCCATGT
GCTTGTCCAGCCACCCGGGCCACCCCACCATCGGCCATGGGTAGG
GCCTCCCCACCGCTCTTTCAGTCCCGCAGCAGCTCGCCCCTGCAG
CTCAACCTGCTGCAGCTGGAGGAAGCCCCTGAGGGTGGCACTGGA
GCCATGGGGACCACAGGGGCCACAGAGACAGCAGCTGTAGGGGCG
GACTGCAAACCTGGCACTTCTCGGGACCAGCAGCCGAAGGCGCCT
CTGACCCGTGATGAACCCTCAGACACACAGAACAGTGACGCCCTT
TCCACGTCAAGCGGCCTCCTAAACCTCCTGCTGAATGAGGACCTC
TGCTCAGCCTCGGGCTCTGCTGCTTCGGAGTCTCTGGGCTCCGGC
TCACTGGGCTGCGACGCCTCCCCGAGTGGGGCAGGCAGTAGTGAC
ACAAGTCATACCAGCAAATATTTTGGAAGCATTGACTCCTCAGAG
AATAATCACAAAGCAAAAATGAACACTGGTATGGAAGAAAGTGAG
CATTTCATTAAGTGCGTCCTGCAGGATCCCATCTGGCTGCTGATG
GCAGATGCGGACAGCAGCGTCATGATGACGTACCAGCTGCCTTCC
CGAAATTTAGAAGCGGTTTTGAAGGAGGACAGAGAGAAGCTGAAG
CTCCTACAGAAACTCCAGCCCAGGTTCACGGAGAGTCAGAAGCAG
GAGCTGCGCGAGGTCCACCAGTGGATGCAGACGGGCGGCCTGCCC
GCAGCCATCGACGTGGCAGAATGTGTTTACTGTGAAAACAAGGAA
AAAGGTAATATTTGCATACCATATGAGGAAGATATTCCTTCTCTG
GGACTCAGCGAAGTGTCGGACACCAAAGAAGACGAAAATGGATCC
CCCTTGAATCACAGGATCGAAGAGCAGACGTAA
The sequence of PER3 cDNA comprises
SEQ ID No. 5:
>ENST00000613533.4 PER3-208 cdna:
protein_coding
ATGCCCCGCGGGGAAGCTCCTGGCCCCGGGAGACGGGGGGCTAAG
GACGAGGCCCTGGGCGAAGAATCGGGGGAGCGGTGGAGCCCCGAG
TTCCATCTGCAGAGGAAATTGGCGGACAGCAGCCACAGTGAACAG
CAAGATCGAAACAGAGTTTCTGAAGAACTTATCATGGTTGTCCAA
GAAATGAAAAAATACTTCCCCTCGGAGAGACGCAATAAACCAAGC
ACTCTAGATGCCCTCAACTATGCTCTCCGCTGTGTCCACAGCGTT
CAAGCAAACAGTGAGTTTTTCCAGATTCTCAGTCAGAATGGAGCA
CCTCAGGCAGATGTGAGCATGTACAGTCTTGAGGAGCTGGCCACT
ATCGCTTCAGAACACACTTCCAAAAACACAGATACCTTTGTGGCA
GTATTTTCATTTCTGTCTGGAAGGTTAGTGCACATTTCTGAACAG
GCTGCTTTGATCCTGAATCGTAAGAAAGATGTCCTGGCGTCTTCT
CACTTTGTTGACCTGCTTGCACCTCAAGACATGAGGGTATTCTAC
GCGCACACTGCCAGAGCTCAGCTTCCTTTCTGGAACAACTGGACC
CAAAGAGCAGCTGCACGGTATGAATGTGCTCCGGTGAAACCTTTT
TTCTGCAGGATCCGTGGAGGTGAAGACAGAAAGCAAGAGAAGTGT
CACTCCCCATTCCGGATCATCCCCTATCTGATTCATGTACATCAC
CCTGCCCAGCCAGAATTGGAATCGGAACCTTGCTGTCTCACTGTG
GTTGAAAAGATTCACTCTGGTTATGAAGCTCCTCGGATCCCAGTG
AATAAAAGAATCTTCACCACCACACACACCCCAGGGTGTGTTTTT
CTTGAAGTAGATGAAAAAGCAGTGCCTTTGCTGGGTTACCTACCT
CAGGACCTGATTGGAACATCGATCCTAAGCTACCTGCACCCTGAA
GATCGTTCTCTGATGGTTGCCATACACCAAAAAGTTTTGAAGTAT
GCAGGGCATCCTCCCTTTGAACATTCTCCCATTCGATTTTGTACT
CAAAACGGAGACTACATCATACTGGATTCCAGTTGGTCCAGCTTT
GTGAATCCCTGGAGCCGGAAGATTTCTTTCATCATTGGTCGGCAT
AAAGTTCGAACGAGCCCACTAAATGAGGATGTTTTTGCTACCAAA
ATTAAAAAGATGAACGATAATGACAAAGACATAACAGAATTACAA
GAACAAATTTACAAACTTCTCTTACAGCCAGTTCACGTGAGCGTG
TCCAGCGGCTACGGGAGCCTGGGGAGCAGCGGGTCGCAGGAGCAG
CTTGTCAGCATCGCCTCCTCCAGTGAGGCCAGTGGGCACCGTGTG
GAGGAGACGAAGGCGGAGCAGATGACCTTGCAGCAGGTCTATGCC
AGTGTGAACAAAATTAAAAATCTGGGTCAGCAGCTCTACATTGAG
TCAATGACCAAATCATCATTCAAGCCAGTGACGGGGACACGCACA
GAACCGAATGGTGGTGGTGAGTCAGCGAATGGTGGTGGTGAATGT
AAGACCTTTACTTCCTTCCACCAAACACTGAAAAACAATAGTGTG
TACACTGAGCCCTGTGAGGATTTGAGGAACGATGAGCACAGCCCA
TCCTATCAACAGATCAACTGTATCGACAGTGTCATCAGATACCTG
AAGAGCTACAACATTCCAGCTTTGAAAAGAAAGTGTATCTCCTGT
ACAAATACAACTTCTTCCTCCTCAGAAGAAGACAAACAGAACCAC
AAGGCAGATGATGTCCAAGCCTTACAAGCTGGTTTGCAAATCCCA
GCCATACCTAAATCAGAAATGCCAACAAATGGACGGTCCATAGAC
ACAGGAGGAGGAGCTCCACAGATCCTGTCCACGGCGATGCTGAGC
TTGGGGTCGGGCATAAGCCAATGCGGTTACAGCAGCACCATTGTC
CATGTCCCACCCCCAGAGACAGCCAGGGATGCTACCCTCTTCTGT
GAGCCCTGGACCCTGAACATGCAGCCAGCCCCTTTGACCTCGGAA
GAATTTAAACACGTGGGGCTCACAGCGGCTGTTCTGTCAGCGCAC
ACCCAGAAGGAAGAGCAGAATTATGTTGATAAATTCCGAGAAAAG
ATCCTGTCATCACCCTACAGCTCCTATCTTCAGCAAGAAAGCAGG
AGCAAAGCTAAATATTCATATTTTCAAGGAGATTCTACTTCCAAG
CAGACGCGGTCGGCCGGCTGCAGGAAAGGGAAGCACAAGCGGAAG
AAGCTGCCGGAGCCGCCAGACAGCAGCAGCTCGAACACCGGCTCT
GGTCCCCGCAGGGGAGCGCATCAGAACGCACAGCCCTGCTGCCCC
TCCGCGGCCTCCTCTCCGCACACCTCGAGCCCGACCTTCCCACCT
GCCGCCATGGTGCCCAGCCAGGCCCCTTACCTCGTCCCAGCTTTT
CCCCTCCCAGCCGCGACCTCACCCGGAAGAGAATACGCAGCCCCC
GGAACTGCACCGGAAGGCCTGCATGGGCTGCCCTTGTCCGAGGGC
TTGCAGCCTTACCCAGCTTTCCCTTTTCCTTACTTGGATACTTTT
ATGACCGTTTTCCTGCCTGACCCCCCTGTCTGTCCTCTGTTGTCG
CCATCGTTTTTGCCATGTCCATTCCTGGGGGCGACAGCCTCTTCT
GCGATATCACCCTCAATGTCGTCAGCAATGAGTCCAACTCTGGAC
CCACCCCCTTCAGTCACCAGCCAAAGGAGAGAGGAGGAAAAGTGG
GAGGCACAAAGCGAGGGGCACCCGTTCATTACTTCGAGAAGCAGC
TCACCCTTGCAGTTAAACTTACTTCAGGAAGAGATGCCCAGACCC
TCTGAATCTCCAGATCAGATGAGAAGGAACACGTGCCCACAAACT
GAGTATCAGTGTGTTACAGGCAACAATGGCAGTGAGAGCAGTCCT
GCTACTACCGGTGCACTGTCCACGGGGTCACCTCCCAGGGAGAAT
CCATCCCATCCTACTGCCAGCGCTCTGTCCACAGGATCGCCTCCC
ATGAAGAATCCATCCCATCCTACTGCCAGCGCTCTGTCCACAGGA
TCGCCTCCCATGAAGAATCCATCCCATCCTACTGCCAGCACACTG
TCCATGGGATTGCCTCCCAGCAGGACTCCATCCCATCCTACTGCC
ACTGTTCTGTCCACGGGGTCACCTCCCAGCGAATCCCCATCCAGA
ACTGGTTCAGCAGCATCAGGAAGCAGCGACAGCAGTATATACCTT
ACTAGTAGTGTTTATTCTTCTAAAATCTCCCAAAATGGGCAGCAA
TCTCAGGACGTACAGAAAAAAGAAACATTTCCTAATGTCGCCGAA
GAGCCCATCTGGAGAATGATACGGCAGACACCTGAGCGCATTCTC
ATGACATACCAGGTACCTGAGAGGGTTAAAGAAGTTGTACTAAAA
GAAGACCTGGAAAAGCTAGAAAGTATGAGGCAGCAGCAGCCCCAG
TTTTCTCATGGGCAAAAGGAGGAGCTGGCTAAGGTGTATAATTGG
ATTCAAAGCCAGACTGTCACTCAAGAAATCGACATTCAAGCCTGT
GTCACTTGTGAAAATGAAGATTCAGCTGATGGTGCGGCCACATCC
TGTGGTCAGGTTCTGGTAGAAGACAGCTGTTGA
The sequence of cDNA CLOCK comprises
SEQ ID No. 6
>ENST00000513440.6 CLOCK-211 cdna:
protein_coding
ATGTTGTTTACCGTAAGCTGTAGTAAAATGAGCTCGATTGTTGAC
AGAGATGACAGTAGTATTTTTGATGGGTTGGTGGAAGAAGATGAC
AAGGACAAAGCGAAAAGAGTATCTAGAAACAAATCTGAAAAGAAA
CGTAGAGATCAATTTAATGTTCTCATTAAAGAACTGGGATCCATG
CTTCCTGGTAATGCTAGAAAGATGGACAAATCTACTGTTCTGCAG
AAAAGCATTGATTTTTTACGAAAACATAAAGAAATCACTGCACAG
TCAGATGCTAGTGAAATTCGACAGGACTGGAAACCTACATTCCTT
AGTAATGAAGAGTTTACACAATTAATGTTAGAGGCTCTTGATGGT
TTTTTTTTAGCAATCATGACAGATGGAAGCATAATATATGTGTCT
GAGAGTGTAACTTCATTACTTGAACATTTACCATCTGATCTTGTG
GATCAAAGTATATTTAATTTTATCCCAGAAGGGGAACATTCAGAG
GTTTATAAAATACTCTCTACTCATCTGCTGGAAAGTGATTCATTA
ACCCCAGAATATTTAAAATCAAAAAATCAGTTAGAATTCTGTTGT
CACATGCTGCGAGGAACAATAGACCCAAAGGAGCCATCTACCTAT
GAATATGTAAAATTTATAGGAAATTTCAAATCTTTAAACAGTGTA
TCCTCTTCAGCACACAATGGTTTTGAAGGAACTATACAACGCACA
CATAGGCCATCTTATGAAGATAGAGTTTGTTTTGTAGCTACTGTC
AGGTTAGCTACACCTCAGTTCATCAAGGAAATGTGCACTGTTGAA
GAACCCAATGAAGAGTTTACATCTAGACATAGTTTAGAATGGAAG
TTTCTGTTTCTAGATCACAGGGCACCACCCATAATAGGGTATTTG
CCATTTGAAGTTCTGGGAACATCAGGCTATGATTACTATCATGTG
GATGACCTAGAAAATTTGGCAAAATGTCATGAGCACTTAATGCAA
TATGGGAAAGGCAAATCATGTTATTATAGGTTCCTGACTAAGGGG
CAACAGTGGATTTGGCTTCAGACTCATTATTATATCACTTACCAT
CAGTGGAATTCAAGGCCAGAGTTTATTGTTTGTACTCACACTGTA
GTAAGTTATGCAGAAGTTAGGGCTGAAAGACGACGAGAACTTGGC
ATTGAAGAGTCTCTTCCTGAGACAGCTGCTGACAAAAGCCAAGAT
TCTGGGTCAGATAATCGTATAAACACAGTCAGTCTCAAGGAAGCA
TTGGAAAGGTTTGATCACAGCCCAACCCCTTCTGCCTCTTCTCGG
AGTTCAAGAAAATCATCTCACACGGCCGTCTCAGACCCTTCCTCA
ACACCAACCAAGATCCCGACGGATACGAGCACTCCACCCAGGCAG
CATTTACCAGCTCATGAGAAGATGGTGCAAAGAAGGTCATCATTT
AGTAGTCAGTCCATAAATTCCCAGTCTGTTGGTTCATCATTAACA
CAGCCAGTGATGTCTCAAGCTACAAATTTACCAATTCCACAAGGC
ATGTCCCAGTTTCAGTTTTCAGCTCAATTAGGAGCCATGCAACAT
CTGAAAGACCAATTGGAACAACGGACACGCATGATAGAAGCAAAT
ATTCATCGGCAACAAGAAGAACTAAGAAAAATTCAAGAACAACTT
CAGATGGTCCATGGTCAGGGGCTGCAGATGTTTTTGCAACAATCA
AATCCTGGGTTGAATTTTGGTTCCGTTCAACTTTCTTCTGGAAAT
TCATCTAATATCCAGCAACTTGCACCTATAAATATGCAAGGCCAA
GTTGTTCCTACTAACCAGATTCAAAGTGGAATGAATACTGGACAC
ATTGGCACAACTCAGCACATGATACAACAACAGACTTTACAGAGT
ACATCAACTCAGAGTCAACAAAATGTACTGAGTGGGCACAGTCAG
CAAACATCTCTACCCAGTCAGACACAGAGCACTCTTACAGCCCCA
CTGTATAACACTATGGTGATTTCTCAGCCTGCAGCCGGAAGCATG
GTCCAGATTCCATCTAGTATGCCACAAAACAGCACCCAGAGTGCT
GCAGTAACTACATTCACTCAGGACAGGCAGATAAGATTTTCTCAA
GGTCAACAACTTGTGACCAAATTAGTGACTGCTCCTGTAGCTTGT
GGGGCAGTCATGGTACCTAGTACTATGCTTATGGGCCAGGTGGTG
ACTGCATATCCTACTTTTGCTACACAACAGCAACAGTCACAGACA
TTGTCAGTAACGCAGCAGCAGCAGCAGCAGAGCTCCCAGGAGCAG
CAGCTCACTTCAGTTCAGCAACCATCTCAGGCTCAGCTGACCCAG
CCACCGCAACAATTTTTACAGACTTCTAGGTTGCTCCATGGGAAT
CCCTCAACTCAACTCATTCTCTCTGCTGCATTTCCTCTACAACAG
AGCACCTTCCCTCAGTCACATCACCAGCAACATCAGTCTCAGCAA
CAGCAGCAACTCAGCCGGCACAGGACTGACAGCTTGCCCGACCCT
TCCAAGGTTCAACCACAGTAG
The sequence of cDNA NPAS2 comprises
SEQ ID No. 7
>ENST00000335681.10 NPAS2-201 cdna:
protein_coding
ATGGATGAAGATGAGAAAGACAGAGCCAAGAGAGCTTCTCGAAAC
AAGTCTGAGAAGAAGCGTCGGGACCAGTTCAATGTTCTCATCAAA
GAGCTCAGTTCCATGCTCCCTGGCAACACGCGGAAAATGGACAAA
ACCACCGTGTTGGAAAAGGTCATCGGATTTTTGCAGAAACACAAT
GAAGTCTCAGCGCAAACGGAAATCTGTGACATTCAGCAAGACTGG
AAGCCTTCATTCCTCAGTAATGAAGAATTCACCCAGCTGATGTTG
GAGGCATTAGATGGCTTCATTATCGCAGTGACAACAGACGGCAGC
ATCATCTATGTCTCTGACAGTATCACGCCTCTCCTTGGGCATTTA
CCGTCGGATGTCATGGATCAGAATTTGTTAAATTTCCTCCCAGAA
CAAGAACATTCAGAAGTTTATAAAATCCTTTCTTCCCATATGCTT
GTGACGGATTCCCCCTCCCCAGAATACTTAAAATCTGACAGCGAT
TTAGAGTTTTATTGCCATCTTCTCAGAGGCAGCTTGAACCCAAAG
GAATTTCCAACTTATGAATACATAAAATTTGTAGGAAATTTTCGC
TCTTACAACAATGTGCCTAGCCCCTCCTGTAATGGTTTTGACAAC
ACCCTTTCAAGACCTTGCCGGGTGCCACTAGGAAAGGAGGTTTGC
TTCATTGCCACCGTTCGTCTGGCAACACCACAATTCTTAAAGGAA
ATGTGCATAGTTGACGAACCTTTAGAGGAATTCACTTCAAGGCAT
AGCTTGGAATGGAAATTTTTATTTCTGGATCACAGAGCACCTCCA
ATCATAGGATACCTGCCTTTTGAAGTGCTGGGAACCTCAGGCTAT
GACTACTACCACATTGATGACCTGGAGCTCCTGGCCAGGTGTCAC
CAGCACCTGATGCAGTTTGGCAAAGGGAAGTCGTGTTGCTACCGG
TTTCTGACCAAAGGTCAGCAGTGGATCTGGCTGCAGACTCACTAC
TACATCACCTACCATCAGTGGAACTCCAAGCCCGAGTTCATCGTG
TGCACACACTCGGTGGTCAGTTACGCAGATGTCCGGGTGGAAAGG
AGGCAGGAGCTGGCTCTGGAAGACCCGCCATCCGAGGCCCTCCAC
TCCTCAGCACTAAAGGACAAGGGCTCAAGCCTGGAACCTCGGCAG
CACTTTAACACACTCGACGTGGGTGCCTCGGGCCTTAATACCAGT
CATTCGCCATCGGCGTCCTCAAGAAGTTCCCACAAATCCTCGCAC
ACAGCCATGTCAGAACCCACCTCCACTCCCACCAAGCTGATGGCA
GAGGCCAGCACCCCGGCTTTGCCAAGATCAGCCACCCTGCCCCAA
GAGTTACCTGTCCCCGGGCTCAGCCAGGCAGCCACCATGCCGGCC
CCTCTGCCTTCCCCATCGTCCTGCGACCTCACACAGCAGCTCCTG
CCTCAGACCGTTCTGCAGAGCACGCCCGCTCCCATGGCACAGTTT
TCGGCACAGTTCAGCATGTTCCAGACCATCAAAGACCAGCTAGAG
CAGCGGACGCGGATCCTGCAGGCCAATATCCGGTGGCAACAGGAA
GAGCTCCACAAGATCCAGGAGCAGCTCTGCCTGGTCCAGGACTCC
AACGTCCAGATGTTCCTGCAGCAGCCAGCTGTATCCCTGAGCTTC
AGCAGCACCCAGCGACCTGAGGCTCAGCAGCAGCTACAGCAAAGG
TCAGCTGCAGTGACTCAGCCCCAGCTCGGGGCGGGCCCCCAACTT
CCAGGGCAGATCTCCTCTGCCCAGGTCACAAGCCAGCACCTGCTC
AGAGAATCAAGTGTGATATCAACCCAGGGTCCAAAGCCAATGAGA
AGCTCACAGCTAATGCAGAGCAGCGGCCGCTCTGGAAGCAGCCTA
GTGTCCCCGTTCAGCAGCGCCACAGCTGCGCTCCCGCCAAGTCTG
AATCTGACCACACCTGCTTCCACCTCCCAGGATGCCAGCCAGTGC
CAGCCCAGCCCAGACTTCAGCCATGATCGGCAGCTCAGGCTGTTG
CTGAGCCAGCCCATCCAGCCCATGATGCCCGGGTCCTGTGACGCA
AGGCAGCCCTCGGAAGTCAGCAGGACGGGACGGCAAGTCAAGTAC
GCCCAGAGCCAGACCGTGTTTCAAAATCCAGACGCACACCCCGCC
AACAGCAGCAGCGCCCCGATGCCCGTCCTGCTGATGGGGCAGGCG
GTGCTCCACCCCAGCTTCCCTGCCTCCCAACCATCGCCCCTGCAG
CCTGCACAGGCCCGGCAGCAGCCACCGCAGCACTACCTGCAGGTA
CAGGCACCAACCTCTTTGCACAGTGAGCAGCAGGACTCGCTACTT
CTCTCCACCTACTCACAACAGCCAGGGACCCTGGGCTACCCCCAA
CCACCCCCAGCACAGCCCCAGCCCCTACGTCCTCCCCGAAGGGTC
AGCAGTCTGTCTGAGTCGTCAGGCCTCCAGCAGCCGCCCCGATAA
The sequence of cDNA CRY1 comprises
SEQ ID No. 8
>ENST00000008527.10 CRY1-201 cdna:
protein_coding
ATGGGGGTGAACGCCGTGCACTGGTTCCGAAAGGGGCTCCGGCTC
CACGACAACCCCGCCCTGAAGGAGTGCATTCAGGGCGCCGACACC
ATCCGCTGCGTCTACATCCTGGACCCCTGGTTCGCCGGCTCCTCC
AATGTGGGCATCAACAGGTGGCGATTTTTGCTTCAGTGTCTTGAG
GATCTTGATGCCAATCTACGAAAATTAAACTCCCGTCTGTTTGTG
ATTCGTGGACAACCAGCAGATGTGTTTCCCAGGCTTTTCAAGGAA
TGGAACATTACTAAACTTTCAATTGAGTATGATTCTGAGCCCTTT
GGAAAGGAACGAGACGCAGCTATTAAGAAACTGGCAACTGAAGCT
GGAGTAGAAGTCATTGTAAGAATTTCACATACATTATATGACCTA
GACAAGATCATAGAACTCAATGGTGGACAACCGCCTCTAACTTAT
AAAAGATTCCAGACTCTCATCAGCAAAATGGAACCACTAGAGATA
CCAGTAGAGACAATTACTTCAGAAGTGATAGAAAAGTGCACAACT
CCTCTGTCTGATGACCATGATGAGAAATATGGAGTCCCTTCACTG
GAAGAGCTAGGTTTTGATACAGATGGCTTATCCTCTGCAGTGTGG
CCAGGTGGAGAAACTGAAGCACTTACTCGTTTGGAAAGGCATTTG
GAAAGAAAAGCTTGGGTGGCAAATTTTGAAAGACCTCGAATGAAT
GCGAATTCTCTGCTTGCAAGCCCTACTGGACTTAGTCCTTATCTC
CGATTTGGTTGTTTGTCATGTCGACTGTTTTACTTCAAACTAACA
GATCTCTACAAAAAGGTAAAGAAGAACAGTTCCCCTCCCCTTTCC
CTTTATGGGCAACTGTTATGGCGTGAATTTTTCTATACAGCAGCA
ACAAATAATCCACGCTTTGATAAAATGGAAGGAAACCCTATCTGT
GTTCAGATTCCTTGGGATAAAAATCCTGAGGCTTTAGCCAAATGG
GCGGAAGGCCGGACAGGCTTTCCATGGATTGATGCCATCATGACA
CAGCTTCGTCAGGAGGGTTGGATTCATCATCTAGCCAGGCATGCA
GTTGCTTGCTTCCTGACACGAGGGGACCTGTGGATTAGTTGGGAA
GAAGGAATGAAGGTATTTGAAGAATTATTGCTTGATGCAGATTGG
AGCATAAATGCTGGAAGTTGGATGTGGCTGTCTTGTAGTTCCTTT
TTTCAACAGTTTTTTCACTGCTATTGCCCTGTTGGTTTTGGTAGG
AGAACAGATCCCAATGGAGACTATATCAGGCGTTATTTGCCTGTC
CTAAGAGGCTTCCCTGCAAAATATATCTATGATCCCTGGAATGCA
CCAGAAGGTATCCAAAAGGTAGCCAAATGTTTGATAGGAGTTAAT
TATCCTAAACCAATGGTGAACCATGCTGAGGCAAGCCGTTTGAAT
ATCGAAAGGATGAAACAGATCTATCAGCAGCTTTCACGATATAGA
GGACTAGGTCTTCTGGCATCAGTACCTTCTAATCCTAATGGGAAT
GGAGGCTTCATGGGATATTCTGCAGAAAATATCCCAGGTTGTAGC
AGCAGTGGAAGTTGCTCTCAAGGGAGTGGTATTTTACACTATGCT
CATGGCGACAGTCAGCAAACTCACCTGTTGAAGCAAGGAAGAAGC
TCCATGGGCACTGGTCTCAGTGGTGGGAAACGTCCTAGTCAGGAA
GAGGACACACAGAGTATTGGTCCTAAAGTCCAGAGACAGAGCACT
AATTAG
The sequence of cDNA CRY2 comprises
SEQ ID No. 9
>ENST00000616623.4 CRY2-212 cdna:
protein_coding
ATGGGCGGGGTCCACGTCGCCTACCGGGGCGGAGCGGGGGTGGCT
GGAGCAGTCTGGACAGTCATGGCGGCGACTGTGGCGACGGCGGCA
GCTGTGGCCCCGGCGCCAGCGCCCGGCACGGACAGCGCCTCTTCG
GTGCACTGGTTCCGCAAAGGGCTGCGACTCCACGACAACCCGGCG
TTGCTGGCGGCCGTGCGCGGGGCGCGCTGCGTGCGCTGCGTTTAC
ATTCTCGACCCGTGGTTCGCGGCCTCCTCCTCAGTCGGGATCAAC
CGATGGAGGTTCCTACTTCAGTCTCTGGAAGATTTGGACACAAGT
TTAAGGAAACTGAACTCCCGCCTGTTTGTAGTCCGGGGACAGCCA
GCCGACGTGTTCCCAAGGCTGTTCAAGGAATGGGGAGTGACCCGC
TTGACCTTTGAATATGACTCTGAACCCTTTGGGAAAGAACGGGAT
GCAGCCATCATGAAGATGGCCAAGGAGGCTGGTGTGGAAGTAGTG
ACGGAGAATTCTCATACCCTCTATGACCTGGACAGGATCATTGAG
CTGAATGGGCAGAAGCCACCCCTTACATACAAGCGCTTTCAGGCC
ATCATCAGCCGCATGGAGCTGCCCAAGAAGCCAGTGGGCTTGGTG
ACCAGCCAGCAGATGGAGAGCTGCAGGGCCGAGATCCAGGAGAAC
CACGACGAGACCTACGGCGTGCCCTCCCTGGAGGAGCTGGGGTTC
CCCACTGAAGGACTTGGTCCAGCTGTCTGGCAGGGAGGAGAGACA
GAAGCTCTGGCCCGCCTGGATAAGCACTTGGAACGGAAGGCCTGG
GTTGCCAACTATGAGAGACCCCGAATGAACGCCAACTCCCTCCTG
GCCAGCCCCACAGGCCTCAGCCCCTACCTGCGCTTTGGTTGTCTC
TCCTGCCGCCTCTTCTACTACCGCCTGTGGGACCTGTATAAAAAG
GTGAAGCGGAACAGCACACCTCCCCTCTCCCTATTTGGGCAACTC
CTATGGCGAGAGTTCTTCTACACGGCAGCTACCAACAACCCCAGG
TTTGACCGCATGGAGGGGAACCCCATCTGCATCCAGATCCCCTGG
GACCGCAATCCTGAGGCCCTGGCCAAGTGGGCTGAGGGCAAGACA
GGCTTCCCTTGGATTGATGCCATCATGACCCAACTGAGGCAGGAG
GGCTGGATCCACCACCTGGCCCGGCATGCCGTGGCCTGCTTCCTG
ACCCGCGGGGACCTCTGGGTCAGCTGGGAGAGCGGGGTCCGGGTA
TTTGATGAGCTGCTCCTGGATGCAGATTTCAGCGTGAACGCAGGC
AGCTGGATGTGGCTGTCCTGCAGTGCTTTCTTCCAGCAGTTCTTC
CACTGCTACTGCCCTGTGGGCTTTGGCCGTCGCACGGACCCCAGT
GGGGACTACATCAGGCGATACCTGCCCAAATTGAAAGCGTTCCCC
TCTCGATACATCTATGAGCCCTGGAATGCCCCAGAGTCAATTCAG
AAGGCAGCCAAGTGCATCATTGGTGTGGACTACCCACGGCCCATC
GTCAACCATGCCGAGACCAGCCGGCTTAACATTGAACGAATGAAG
CAGATTTACCAGCAGCTTTCGCGCTACCGGGGACTCTGTCTACTG
GCATCTGTCCCTTCCTGTGTGGAAGACCTCAGTCACCCTGTGGCA
GAGCCCAGCTCGAGCCAGGCTGGCAGCATGAGCAGTGCAGGCCCA
AGACCACTACCCAGTGGCCCAGCATCCCCCAAACGCAAGCTGGAA
GCAGCCGAGGAACCACCTGGTGAAGAACTCAGCAAACGGGCCCGG
GTGGCAGAGTTGCCAACCCCAGAGCTGCCGAGCAAGGATGCCTGA
The sequence of cDNA NR1D1comprises
SEQ ID No. 10
>ENST00000246672.4 NR1D1-201 cdna:
protein_coding
ATGACGACCCTGGACTCCAACAACAACACAGGTGGCGTCATCACC
TACATTGGCTCCAGTGGCTCCTCCCCAAGCCGCACCAGCCCTGAA
TCCCTCTATAGTGACAACTCCAATGGCAGCTTCCAGTCCCTGACC
CAAGGCTGTCCCACCTACTTCCCACCATCCCCCACTGGCTCCCTC
ACCCAAGACCCGGCTCGCTCCTTTGGGAGCATTCCACCCAGCCTG
AGTGATGACGGCTCCCCTTCTTCCTCATCTTCCTCGTCGTCATCC
TCCTCCTCCTTCTATAATGGGAGCCCCCCTGGGAGTCTACAAGTG
GCCATGGAGGACAGCAGCCGAGTGTCCCCCAGCAAGAGCACCAGC
AACATCACCAAGCTGAATGGCATGGTGTTACTGTGTAAAGTGTGT
GGGGACGTTGCCTCGGGCTTCCACTACGGTGTGCACGCCTGCGAG
GGCTGCAAGGGCTTTTTCCGTCGGAGCATCCAGCAGAACATCCAG
TACAAAAGGTGTCTGAAGAATGAGAATTGCTCCATCGTCCGCATC
AATCGCAACCGCTGCCAGCAATGTCGCTTCAAGAAGTGTCTCTCT
GTGGGCATGTCTCGAGACGCTGTGCGTTTTGGGCGCATCCCCAAA
CGAGAGAAGCAGCGGATGCTTGCTGAGATGCAGAGTGCCATGAAC
CTGGCCAACAACCAGTTGAGCAGCCAGTGCCCGCTGGAGACTTCA
CCCACCCAGCACCCCACCCCAGGCCCCATGGGCCCCTCGCCACCC
CCTGCTCCGGTCCCCTCACCCCTGGTGGGCTTCTCCCAGTTTCCA
CAACAGCTGACGCCTCCCAGATCCCCAAGCCCTGAGCCCACAGTG
GAGGATGTGATATCCCAGGTGGCCCGGGCCCATCGAGAGATCTTC
ACCTACGCCCATGACAAGCTGGGCAGCTCACCTGGCAACTTCAAT
GCCAACCATGCATCAGGTAGCCCTCCAGCCACCACCCCACATCGC
TGGGAAAATCAGGGCTGCCCACCTGCCCCCAATGACAACAACACC
TTGGCTGCCCAGCGTCATAACGAGGCCCTAAATGGTCTGCGCCAG
GCTCCCTCCTCCTACCCTCCCACCTGGCCTCCTGGCCCTGCACAC
CACAGCTGCCACCAGTCCAACAGCAACGGGCACCGTCTATGCCCC
ACCCACGTGTATGCAGCCCCAGAAGGCAAGGCACCTGCCAACAGT
CCCCGGCAGGGCAACTCAAAGAATGTTCTGCTGGCATGTCCTATG
AACATGTACCCGCATGGACGCAGTGGGCGAACGGTGCAGGAGATC
TGGGAGGATTTCTCCATGAGCTTCACGCCCGCTGTGCGGGAGGTG
GTAGAGTTTGCCAAACACATCCCGGGCTTCCGTGACCTTTCTCAG
CATGACCAAGTCACCCTGCTTAAGGCTGGCACCTTTGAGGTGCTG
ATGGTGCGCTTTGCTTCGTTGTTCAACGTGAAGGACCAGACAGTG
ATGTTCCTAAGCCGCACCACCTACAGCCTGCAGGAGCTTGGTGCC
ATGGGCATGGGAGACCTGCTCAGTGCCATGTTCGACTTCAGCGAG
AAGCTCAACTCCCTGGCGCTTACCGAGGAGGAGCTGGGCCTCTTC
ACCGCGGTGGTGCTTGTCTCTGCAGACCGCTCGGGCATGGAGAAT
TCCGCTTCGGTGGAGCAGCTCCAGGAGACGCTGCTGCGGGCTCTT
CGGGCTCTGGTGCTGAAGAACCGGCCCTTGGAGACTTCCCGCTTC
ACCAAGCTGCTGCTCAAGCTGCCGGACCTGCGGACCCTGAACAAC
ATGCATTCCGAGAAGCTGCTGTCCTTCCGGGTGGACGCCCAGTGA
The sequence of cDNA NR1D2 comprises
SEQ ID No. 11
>ENST00000312521.9 NR1D2-201 cdna:
protein_coding
ATGGAGGTGAATGCAGGAGGTGTGATTGCCTATATCAGTTCTTCC
AGCTCAGCCTCAAGCCCTGCCTCTTGTCACAGTGAGGGTTCTGAG
AATAGTTTCCAGTCCTCCTCCTCTTCTGTTCCATCTTCTCCAAAT
AGCTCTAATTCTGATACCAATGGTAATCCCAAGAATGGTGATCTC
GCCAATATTGAAGGCATCTTGAAGAATGATCGAATAGATTGTTCT
ATGAAAACAAGCAAATCGAGTGCACCTGGGATGACAAAAAGTCAT
AGTGGTGTGACAAAATTTAGTGGCATGGTTCTACTGTGTAAAGTC
TGTGGGGATGTGGCGTCAGGATTCCACTATGGAGTTCATGCTTGC
GAAGGCTGTAAGGGTTTCTTTCGGAGAAGTATTCAACAAAACATC
CAGTACAAGAAGTGCCTGAAGAATGAAAACTGTTCTATAATGAGA
ATGAATAGGAACAGATGTCAGCAATGTCGCTTCAAAAAGTGTCTG
TCTGTTGGAATGTCAAGAGATGCTGTTCGGTTTGGTCGTATTCCT
AAGCGTGAAAAACAGAGGATGCTAATTGAAATGCAAAGTGCAATG
AAGACCATGATGAACAGCCAGTTCAGTGGTCACTTGCAAAATGAC
ACATTAGTAGAACATCATGAACAGACAGCCTTGCCAGCCCAGGAA
CAGCTGCGACCCAAGCCCCAACTGGAGCAAGAAAACATCAAAAGC
TCTTCTCCTCCATCTTCTGATTTTGCAAAGGAAGAAGTGATTGGC
ATGGTGACCAGAGCTCACAAGGATACCTTTATGTATAATCAAGAG
CAGCAAGAAAACTCAGCTGAGAGCATGCAGCCCCAGAGAGGAGAA
CGGATTCCCAAGAACATGGAGCAATATAATTTAAATCATGATCAT
TGCGGCAATGGGCTTAGCAGCCATTTTCCCTGTAGTGAGAGCCAG
CAGCATCTCAATGGACAGTTCAAAGGGAGGAATATAATGCATTAC
CCAAATGGTCATGCCATTTGTATTGCAAATGGACATTGTATGAAC
TTCTCCAATGCTTATACTCAAAGAGTATGTGATAGAGTTCCGATA
GATGGATTTTCTCAGAATGAGAACAAGAATAGTTACCTGTGCAAC
ACTGGAGGAAGAATGCATCTGGTTTGTCCAATGAGTAAGTCTCCA
TATGTGGATCCTCATAAATCAGGACATGAAATCTGGGAAGAATTT
TCGATGAGCTTCACTCCAGCAGTGAAAGAAGTGGTGGAATTTGCA
AAGCGTATTCCTGGGTTCAGAGATCTCTCTCAGCATGACCAGGTC
AACCTTTTAAAGGCTGGGACTTTTGAGGTTTTAATGGTACGGTTC
GCATCATTATTTGATGCAAAGGAACGTACTGTCACCTTTTTAAGT
GGAAAGAAATATAGTGTGGATGATTTACACTCAATGGGAGCAGGG
GATCTGCTAAACTCTATGTTTGAATTTAGTGAGAAGCTAAATGCC
CTCCAACTTAGTGATGAAGAGATGAGTTTGTTTACAGCTGTTGTC
CTGGTATCTGCAGATCGATCTGGAATAGAAAACGTCAACTCTGTG
GAGGCTTTGCAGGAAACTCTCATTCGTGCACTAAGGACCTTAATA
ATGAAAAACCATCCAAATGAGGCCTCTATTTTTACAAAACTGCTT
CTAAAGTTGCCAGATCTTCGATCTTTAAACAACATGCACTCTGAG
GAGCTCTTGGCCTTTAAAGTTCACCCTTAA
The sequence of cDNA RORA comprises
SEQ ID No. 12
>ENST00000335670.11 RORA-203 cdna:
protein_coding
ATGGAGTCAGCTCCGGCAGCCCCCGACCCCGCCGCCAGCGAGCCA
GGCAGCAGCGGCGCGGACGCGGCCGCCGGCTCCAGGGAGACCCCG
CTGAACCAGGAATCCGCCCGCAAGAGCGAGCCGCCTGCCCCGGTG
CGCAGACAGAGCTATTCCAGCACCAGCAGAGGTATCTCAGTAACG
AAGAAGACACATACATCTCAAATTGAAATTATTCCATGCAAGATC
TGTGGAGACAAATCATCAGGAATCCATTATGGTGTCATTACATGT
GAAGGCTGCAAGGGCTTTTTCAGGAGAAGTCAGCAAAGCAATGCC
ACCTACTCCTGTCCTCGTCAGAAGAACTGTTTGATTGATCGAACC
AGTAGAAACCGCTGCCAACACTGTCGATTACAGAAATGCCTTGCC
GTAGGGATGTCTCGAGATGCTGTAAAATTTGGCCGAATGTCAAAA
AAGCAGAGAGACAGCTTGTATGCAGAAGTACAGAAACACCGGATG
CAGCAGCAGCAGCGCGACCACCAGCAGCAGCCTGGAGAGGCTGAG
CCGCTGACGCCCACCTACAACATCTCGGCCAACGGGCTGACGGAA
CTTCACGACGACCTCAGTAACTACATTGACGGGCACACCCCTGAG
GGGAGTAAGGCAGACTCCGCCGTCAGCAGCTTCTACCTGGACATA
CAGCCTTCCCCAGACCAGTCAGGTCTTGATATCAATGGAATCAAA
CCAGAACCAATATGTGACTACACACCAGCATCAGGCTTCTTTCCC
TACTGTTCGTTCACCAACGGCGAGACTTCCCCAACTGTGTCCATG
GCAGAATTAGAACACCTTGCACAGAATATATCTAAATCGCATCTG
GAAACCTGCCAATACTTGAGAGAAGAGCTCCAGCAGATAACGTGG
CAGACCTTTTTACAGGAAGAAATTGAGAACTATCAAAACAAGCAG
CGGGAGGTGATGTGGCAATTGTGTGCCATCAAAATTACAGAAGCT
ATACAGTATGTGGTGGAGTTTGCCAAACGCATTGATGGATTTATG
GAACTGTGTCAAAATGATCAAATTGTGCTTCTAAAAGCAGGTTCT
CTAGAGGTGGTGTTTATCAGAATGTGCCGTGCCTTTGACTCTCAG
AACAACACCGTGTACTTTGATGGGAAGTATGCCAGCCCCGACGTC
TTCAAATCCTTAGGTTGTGAAGACTTTATTAGCTTTGTGTTTGAA
TTTGGAAAGAGTTTATGTTCTATGCACCTGACTGAAGATGAAATT
GCATTATTTTCTGCATTTGTACTGATGTCAGCAGATCGCTCATGG
CTGCAAGAAAAGGTAAAAATTGAAAAACTGCAACAGAAAATTCAG
CTAGCTCTTCAACACGTCCTACAGAAGAATCACCGAGAAGATGGA
ATACTAACAAAGTTAATATGCAAGGTGTCTACCTTAAGAGCCTTA
TGTGGACGACATACAGAAAAGCTAATGGCATTTAAAGCAATATAC
CCAGACATTGTGCGACTTCATTTTCCTCCATTATACAAGGAGTTG
TTCACTTCAGAATTTGAGCCAGCAATGCAAATTGATGGGTAA
The sequence of cDNA RORB comprises
SEQ ID No. 13
>ENST00000376896.8 RORB-201 cdna:
protein_coding
ATGCGAGCACAAATTGAAGTGATACCATGCAAAATTTGTGGCGAT
AAGTCCTCTGGGATCCACTACGGAGTCATCACATGTGAAGGCTGC
AAGGGATTCTTTAGGAGGAGCCAGCAGAACAATGCTTCTTATTCC
TGCCCAAGGCAGAGAAACTGTTTAATTGACAGAACGAACAGAAAC
CGTTGCCAACACTGCCGACTGCAGAAGTGTCTTGCCCTAGGAATG
TCAAGAGATGCTGTGAAGTTTGGGAGGATGTCCAAGAAGCAAAGG
GACAGCCTGTATGCTGAGGTGCAGAAGCACCAGCAGCGGCTGCAG
GAACAGCGGCAGCAGCAGAGTGGGGAGGCAGAAGCCCTTGCCAGG
GTGTACAGCAGCAGCATTAGCAACGGCCTGAGCAACCTGAACAAC
GAGACCAGCGGCACTTATGCCAACGGGCACGTCATTGACCTGCCC
AAGTCTGAGGGTTATTACAACGTCGATTCCGGTCAGCCGTCCCCT
GATCAGTCAGGACTTGACATGACTGGAATCAAACAGATAAAGCAA
GAACCTATCTATGACCTCACATCCGTACCCAACTTGTTTACCTAT
AGCTCTTTCAACAATGGGCAGTTAGCACCAGGGATAACCATGACT
GAAATCGACCGAATTGCACAGAACATCATTAAGTCCCATTTGGAG
ACATGTCAATACACCATGGAAGAGCTGCACCAGCTGGCGTGGCAG
ACCCACACCTATGAAGAAATTAAAGCATATCAAAGCAAGTCCAGG
GAAGCACTGTGGCAACAATGTGCCATCCAGATCACTCACGCCATC
CAATACGTGGTGGAGTTTGCAAAGCGGATAACAGGCTTCATGGAG
CTCTGTCAAAATGATCAAATTCTACTTCTGAAGTCAGGTTGCTTG
GAAGTGGTTTTAGTGAGAATGTGCCGTGCCTTCAACCCATTAAAC
AACACTGTTCTGTTTGAAGGAAAATATGGAGGAATGCAAATGTTC
AAAGCCTTAGGTTCTGATGACCTAGTGAATGAAGCATTTGACTTT
GCAAAGAATTTGTGTTCCTTGCAGCTGACCGAGGAGGAGATCGCT
TTGTTCTCATCTGCTGTTCTGATATCTCCAGACCGAGCCTGGCTT
ATAGAACCAAGGAAAGTCCAGAAGCTTCAGGAAAAAATTTATTTT
GCACTTCAACATGTGATTCAGAAGAATCACCTGGATGATGAGACC
TTGGCAAAGTTAATAGCCAAGATACCAACCATCACGGCAGTTTGC
AACTTGCACGGGGAGAAGCTGCAGGTATTTAAGCAATCTCATCCA
GAGATAGTGAATACACTGTTTCCTCCGTTATACAAGGAGCTCTTT
AATCCTGACTGTGCCACCGGCTGCAAATGA
The sequence of cDNA RORC comprises
SEQ ID No. 14
>ENST00000318247.7 RORC-201 cdna:
protein_coding
ATGGACAGGGCCCCACAGAGACAGCACCGAGCCTCACGGGAGCTG
CTGGCTGCAAAGAAGACCCACACCTCACAAATTGAAGTGATCCCT
TGCAAAATCTGTGGGGACAAGTCGTCTGGGATCCACTACGGGGTT
ATCACCTGTGAGGGGTGCAAGGGCTTCTTCCGCCGGAGCCAGCGC
TGTAACGCGGCCTACTCCTGCACCCGTCAGCAGAACTGCCCCATC
GACCGCACCAGCCGAAACCGATGCCAGCACTGCCGCCTGCAGAAA
TGCCTGGCGCTGGGCATGTCCCGAGATGCTGTCAAGTTCGGCCGC
ATGTCCAAGAAGCAGAGGGACAGCCTGCATGCAGAAGTGCAGAAA
CAGCTGCAGCAGCGGCAACAGCAGCAACAGGAACCAGTGGTCAAG
ACCCCTCCAGCAGGGGCCCAAGGAGCAGATACCCTCACCTACACC
TTGGGGCTCCCAGACGGGCAGCTGCCCCTGGGCTCCTCGCCTGAC
CTGCCTGAGGCTTCTGCCTGTCCCCCTGGCCTCCTGAAAGCCTCA
GGCTCTGGGCCCTCATATTCCAACAACTTGGCCAAGGCAGGGCTC
AATGGGGCCTCATGCCACCTTGAATACAGCCCTGAGCGGGGCAAG
GCTGAGGGCAGAGAGAGCTTCTATAGCACAGGCAGCCAGCTGACC
CCTGACCGATGTGGACTTCGTTTTGAGGAACACAGGCATCCTGGG
CTTGGGGAACTGGGACAGGGCCCAGACAGCTACGGCAGCCCCAGT
TTCCGCAGCACACCGGAGGCACCCTATGCCTCCCTGACAGAGATA
GAGCACCTGGTGCAGAGCGTCTGCAAGTCCTACAGGGAGACATGC
CAGCTGCGGCTGGAGGACCTGCTGCGGCAGCGCTCCAACATCTTC
TCCCGGGAGGAAGTGACTGGCTACCAGAGGAAGTCCATGTGGGAG
ATGTGGGAACGGTGTGCCCACCACCTCACCGAGGCCATTCAGTAC
GTGGTGGAGTTCGCCAAGAGGCTCTCAGGCTTTATGGAGCTCTGC
CAGAATGACCAGATTGTGCTTCTCAAAGCAGGAGCAATGGAAGTG
GTGCTGGTTAGGATGTGCCGGGCCTACAATGCTGACAACCGCACG
GTCTTTTTTGAAGGCAAATACGGTGGCATGGAGCTGTTCCGAGCC
TTGGGCTGCAGCGAGCTCATCAGCTCCATCTTTGACTTCTCCCAC
TCCCTAAGTGCCTTGCACTTTTCCGAGGATGAGATTGCCCTCTAC
ACAGCCCTTGTTCTCATCAATGCCCATCGGCCAGGGCTCCAAGAG
AAAAGGAAAGTAGAACAGCTGCAGTACAATCTGGAGCTGGCCTTT
CATCATCATCTCTGCAAGACTCATCGCCAAAGCATCCTGGCAAAG
CTGCCACCCAAGGGGAAGCTTCGGAGCCTGTGTAGCCAGCATGTG
GAAAGGCTGCAGATCTTCCAGCACCTCCACCCCATCGTGGTCCAA
GCCGCTTTCCCTCCACTCTACAAGGAGCTCTTCAGCACTGAAACC
GAGTCACCTGTGGGGCTGTCCAAGTGA

In one embodiment of the method according to the invention assessing the circadian rhythm of said subject comprises determining a periodic function for each of at least two core clock genes, in particular for said at least two members of the groups comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, that approximates said expression levels for each of at least two core clock genes, in particular for said at least two members of the groups comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, preferably comprising curve fitting of a non-linear periodic model function to the respective expression levels, wherein the curve fitting is preferably carried out by means of harmonic regression.

The core clock genes may be selected from the group comprising Arntl (Brnall), Arntl2, Clock, Per1, Per2, Per3, Npas2, Cry1, Cry2, Nrld1, Nrld2, Rora, Rorb and Rorc.

In one embodiment of the method according to the invention assessing the circadian rhythm of said subject comprises determining a periodic function for each of ARNTL (BMAL1) and PER2 that approximates said expression levels for each of ARNTL (BMAL1) and PER2, preferably comprising curve fitting of a non-linear periodic model function to the respective expression levels, wherein the curve fitting is preferably carried out by means of harmonic regression.

It will be appreciated that other curve fitting methods may be applied for determining a mathematical function that has the best fit to the series of the measured gene expressions (here: expression levels for each of e.g. ARNTL (BMAL1) and PER2. The skilled person will understand that curve fitting in the context of this disclosure aims at finding a periodic function (oscillatory function) because of the periodicity of the circadian clock(s). While curve fitting may generally aim at finding an interpolation for exact fitting of the data points, methods that approximate the series of measure gene expressions will be preferred, e.g. smoothing, in which a “smooth” function is constructed that approximately fits the data.

It has been found that regression analysis methods are more appropriate here, which use statistical data, not least because the determined periodic function shall represent not only the measured data points but particularly future values. Polynomial interpolation or polynomial regression may be alternatively applied. Preferably, harmonic regression is used, which is based on the trigonometric functions sine and cosine. As will be appreciated by a person skilled in the art, various methods for minimizing an error between the fitted curve and the measured data points may be applied, such as square errors, which is set forth in more detail below. In the method of harmonic regression, the model y(t)=m+a·cos(ωt)+b·sin(ωt) is fitted to the measured data to determine the absolute (A==√(a2+b2)) and relative amplitude as well as the phase (tan φ=b/a), the p-value and the confidence interval. The significance level p may be selected as p<0.05.

In one embodiment of the method according to the invention the computational step comprises processing the determined expression levels and/or the respectively fitted periodic functions to derive characteristic data for each of at least two core clock genes, in particular of said at least two members of the groups comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, said processing comprising determining the mean expression level of expression of at least two core clock genes, in particular of said at least two members of the groups comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, and normalizing the expression levels using the mean expression level.

In one embodiment of the method according to the invention the computational step comprises processing the determined expression levels and/or the respectively fitted periodic functions to derive characteristic data for each of ARNTL (BMAL1), and PER2, said processing comprising determining the mean expression level of expression of ARNTL (BMAL1), and PER2 and normalizing the expression levels using the mean expression level.

Particularly in view of the machine learning processes as described below, the “raw data”, i.e. the measured gene expression levels for each the core clock genes, e.g. of ARNTL (BMAL1), and PER2, including the obtained periodic functions resulting from the curve fitting, have to be preprocessed to bring them into a form that is suitable for the intended machine learning algorithm. For instance, the preprocessing includes extracting data of interest (characteristic data) and setting the dimensionality for the machine learning, i.e. number of parameters. Further, in order to get comparable parameters, normalization is typically required to achieve a common scale for all parameters. It has been found that using the mean expression level for normalizing the measured data is a suitable approach. Further, in order not to lose the absolute values, the mean level is added to the parameter space. This will be set forth also in more detail below.

In one embodiment of the method according to the invention said characteristic data comprise:
the amplitude of change of expression of a gene, and/or the amplitude relative to one of the other genes, and/or
the mean expression level of expression of a gene, and/or the mean relative to one of the other genes, and/or
the peak expression level of a gene, and/or the peak relative to one of the other genes, and/or
the amplitude of change of expression of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC over the day, and/or
the relative difference of the amplitudes of change of expression of any two of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC, and/or
the mean expression level of expression of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC, and/or
the relative difference of the mean expression levels of expression of any two of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC, and/or
the peak expression level of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC over the day, and/or
the relative difference of the peak expression levels of any two of ARNTL (BMAL1) and/or ARNTL2 and/or CLOCK, and/or NPAS2 and/or PER1 and/or PER2 and/or PER3 and/or CRY1 and/or CRY2 and/or NR and/or NR and/or RORA and/or RORB and/or RORC, and/or the time of the peak expression level of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC,
the relative difference of the times of the peak expression level of any two of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC,
wherein the amplitude, period and phase expression level of expression of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC are extracted from the determined expression levels and/or the respectively fitted periodic function.

In one embodiment of the method according to the invention said characteristic data comprise:
the amplitude of change of expression of ARNTL (BMAL1) and/or PER2 over the day, and/or
the mean expression level of expression of ARNTL (BMAL1) and/or PER2, and/or
the peak expression level of ARNTL (BMAL1) and/or PER2 over the day, and/or
the relative expression levels of expression of ARNTL (BMAL1) and PER2, and/or
the time of the peak expression level of ARNTL (BMAL1) and/or PER2, and/or the difference in time of the peak expression levels of ARNTL (BMAL1) and PER2.

The amplitude, period and phase expression level of expression of ARNTL (BMAL1) and/or PER2 are extracted from the determined expression levels and/or the respectively fitted periodic function.

In one embodiment of the method according to the invention of said characteristic data only the timing of the peak expression level of PER2 and the mean expression level of ARNTL (BMAL1) are used in said computational step.

In one embodiment of the method according to the invention the computational step further comprises
fitting a network computational model to the derived characteristic data that comprises a representation of the periodic time course of the expression levels for each of at least two core clock genes, in particular of said at least two members of the groups comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2 as well as a representation of the periodic time course of the expression level for at least one, preferably a plurality of further gene(s) included in a gene regulatory network that includes BMAL1 and PER2; and/or
training a machine learning algorithm on the derived characteristic data of the network computational model, particularly optimize in terms of the representation of the periodic time course of the expression level for the at least one further gene.

In particular, the network computational model is built to obtain data for at least one further gene that has not been directly measured in the saliva samples, i.e. the network computational model represents a gene network which contains the clock elements (those genes of the aforementioned group of core clock genes, which are measured, such as ARNTL (BMAL1) and PER2 and further elements relevant for determining the peak time for sport performance, which further elements cannot (or at least not with reasonable effort) be measured particularly in the saliva samples. This mathematical modelling may use differential equations and also statistical data.

In one embodiment of the method according to the invention assessing and/or predicting the individual diurnal athletic performance times comprises in the computational step fitting a prediction computational model on data obtained from said fitted periodic functions and/or said network computational model, wherein the prediction computational model is based on machine learning, including at least one classification method and/or at least one clustering method wherein said method(s) are preferably selected from the group comprising: K-nearest neighbor algorithm, unsupervised clustering, deep neural networks, random forest algorithm, and support vector machines.

In one embodiment of the method according to the present invention assessing and/or predicting the individual diurnal athletic performance times comprises in the computational step:
Allocating a time-dependent numerical value to said gene expressions corresponding to the respective expression levels of said genes, said genes particularly including ARNTL (BMAL1), ARNTL2, CLOCK, PER1, PER2, PER3, NPAS2, CRY1, CRY2, NR1D1, NR1D2, RORA, RORB, RORC; and
Selecting the optimal and/or non-optimal time of assessing and/or predicting the individual diurnal athletic performance times based on a calculation result using said allocated time-dependent numerical values, wherein a ratio of said time-dependent numerical values for a particular subject is determined, particularly using the network computational model, wherein then, particularly applying the prediction computational model, a minimum and/or maximum of said determined ratios indicates said optimal and/or non-optimal time of assessing and/or predicting the individual diurnal athletic performance times or a range of said determined ratio indicates a period of the day indicating said optimal and/or non-optimal time of assessing and/or predicting the individual diurnal athletic performance times.

In one embodiment of the method according to the invention additional physiological data of the subject are provided for fitting the prediction computational model. Said physiological data may be selected from the group comprising: body temperature, heart rate, eating/fasting patterns and/or sleep/wake patterns. It will be appreciated that one or more of the aforementioned physiological data or other physiological parameters from the subject may be provided. While such data may be obtained manually by the subject (user) and/or by medical staff, it may be envisioned to obtain at least some of the physiological data by means of a portable electronic device, particularly a wearable, such as a fitness watch, wristband or the like. Vice versa, the result of the method of the present invention may be presented on such wearable device so that the user directly sees e.g. their circadian profile (just like they are used to see other physiological or fitness data, e.g. how long and how fast their jogging was, or how their sleep quality was). Of course, the result may be provided by other electronic devices, like a smartphone, tablet or personal computer.

In one embodiment of the method according to the invention the oscillation amplitude and/or peak time of the individual diurnal athletic performance during the day are assessed and/or predicted, wherein predicting the peak time of the individual diurnal athletic performance preferably comprises selecting at least one period of time from at least two distinct periods of time during the day as the peak time. This simple approach may allow determining the peak performance peak at least in two “categories” (i.e. periods of time), such as “early” or “morning” and “late” or “afternoon”/“evening”. Depending on the available data, i.e. the power of the prediction computation model, more precise predictions may be envisioned, e.g. selecting between more (and shorter) time windows per day, specific “peak hours” or even specific points in time that enable the subject to even more precisely select a time for a work out, training or the like.

In one embodiment of the method according to the invention the network computational model and/or the prediction computational model form a personalized model for said subject. The personalization particularly comes from the molecular data, i.e. the measurements of the gene expression which are unique for each person. Additional physiological data like temperature, heart rate, sleep/wake cycles as mentioned above can also be used for personalization. These are all circadian events, too, meaning they vary within 24 hours. While such physiological data may be of additional value, the models, and thus predictions are primarily based on the molecular data, i.e. the gene expressions. It is noted that while the network computational model may be personalized because there is a new model for each new person (using the personal gene expressions), the prediction computational model is not. There is one prediction model relating subject data to prediction for all subjects, and this model should generalize to future subjects without being retrained. This means that, a model for the gene network is built individually for each person, and also each person gets his or her own and individual prediction for the sport peak performance time but using the same model here for each person. However, when using the differential equations model, each new person gets a new model.

Again, a major aspect of the present invention is the personalization of the model. An ODE (ordinary differential equation) model may be used as explained in further detail below. The model may include biological information in it, and predictions on the individual level. Personalization and predictions may be performed beyond circadian time, plus the network is used as described below.

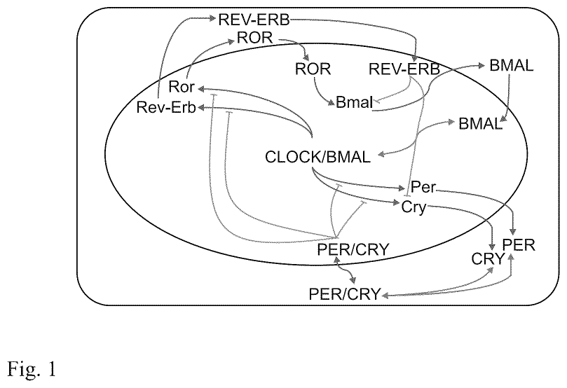

Known models may use machine learning on the harmonic regression, while in contrast the present invention uses an ODE model, which includes additional biological knowledge, as shown in FIG. 1. The computational network allows us to use for prediction derived markers that are informative from one human to the next, despite large differences in their gene expression. The PER2 peak might be such a marker. Markers may be hidden in the actual gene expression, but might result from the dynamic interplay.

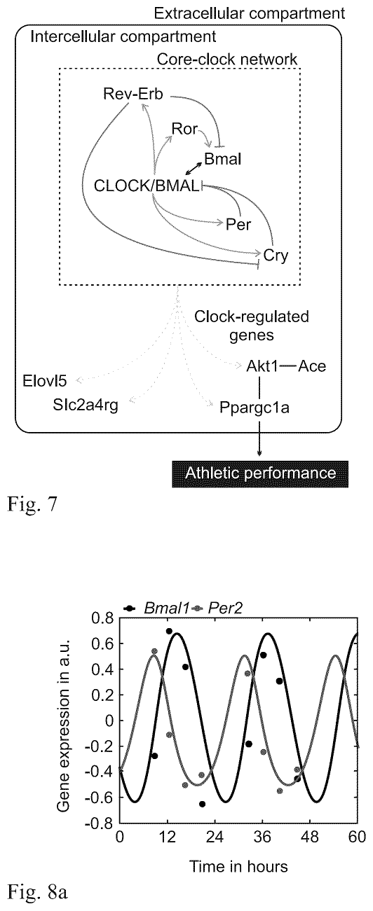

Moreover, the transcription translation networks of the present invention (such as shown in FIG. 1 and FIG. 7) may contain biological information, both regarding the connections of the network, as well as the baseline parameter fit to a representative mammalian tissue (the fit of the saliva is a variation of that baseline model, with a subset of parameters freed for fitting). In contrast to that, previously used models such as simple phase oscillator model, i.e. a phase response curve, are only descriptive (the biological information is restricted to the information that light can shift circadian rhythms). The ODE model of the present invention has several elements, which can be fitted to experimental data. A model fit might even allow to compensate potential methodological errors in the saliva measurements, which would hardly be possible with much simpler models.

In one embodiment of the method according to the invention in addition the expression levels of at least one gene selected from the group comprising AKT1, MYOD1, ACE, PPARGC1A, Elov15 and Slc2a4 is determined or predicted base on a model of the underlying genetic network and used for said assessment and/or prediction.

The sequence of AKT1 comprises
SEQ ID No. 15:
>ENST00000554581.5 AKT1-208 cdna:
protein_coding
ATGAGCGACGTGGCTATTGTGAAGGAGGGTTGGCTGCACAAACGA
GGGGAGTACATCAAGACCTGGCGGCCACGCTACTTCCTCCTCAAG
AATGATGGCACCTTCATTGGCTACAAGGAGCGGCCGCAGGATGTG
GACCAACGTGAGGCTCCCCTCAACAACTTCTCTGTGGCGCAGTGC
CAGCTGATGAAGACGGAGCGGCCCCGGCCCAACACCTTCATCATC
CGCTGCCTGCAGTGGACCACTGTCATCGAACGCACCTTCCATGTG
GAGACTCCTGAGGAGCGGGAGGAGTGGACAACCGCCATCCAGACT
GTGGCTGACGGCCTCAAGAAGCAGGAGGAGGAGGAGATGGACTTC
CGGTCGGGCTCACCCAGTGACAACTCAGGGGCTGAAGAGATGGAG
GTGTCCCTGGCCAAGCCCAAGCACCGCGTGACCATGAACGAGTTT
GAGTACCTGAAGCTGCTGGGCAAGGGCACTTTCGGCAAGGTGATC
CTGGTGAAGGAGAAGGCCACAGGCCGCTACTACGCCATGAAGATC
CTCAAGAAGGAAGTCATCGTGGCCAAGGACGAGGTGGCCCACACA
CTCACCGAGAACCGCGTCCTGCAGAACTCCAGGCACCCCTTCCTC
ACAGCCCTGAAGTACTCTTTCCAGACCCACGACCGCCTCTGCTTT
GTCATGGAGTACGCCAACGGGGGCGAGCTGTTCTTCCACCTGTCC
CGGGAGCGTGTGTTCTCCGAGGACCGGGCCCGCTTCTATGGCGCT
GAGATTGTGTCAGCCCTGGACTACCTGCACTCGGAGAAGAACGTG
GTGTACCGGGACCTCAAGCTGGAGAACCTCATGCTGGACAAGGAC
GGGCACATTAAGATCACAGACTTCGGGCTGTGCAAGGAGGGGATC
AAGGACGGTGCCACCATGAAGACCTTTTGCGGCACACCTGAGTAC
CTGGCCCCCGAGGTGCTGGAGGACAATGACTACGGCCGTGCAGTG
GACTGGTGGGGGCTGGGCGTGGTCATGTACGAGATGATGTGCGGT
CGCCTGCCCTTCTACAACCAGGACCATGAGAAGCTTTTTGAGCTC
ATCCTCATGGAGGAGATCCGCTTCCCGCGCACGCTTGGTCCCGAG
GCCAAGTCCTTGCTTTCAGGGCTGCTCAAGAAGGACCCCAAGCAG
AGGCTTGGCGGGGGCTCCGAGGACGCCAAGGAGATCATGCAGCAT
CGCTTCTTTGCCGGTATCGTGTGGCAGCACGTGTACGAGAAGAAG
CTCAGCCCACCCTTCAAGCCCCAGGTCACGTCGGAGACTGACACC
AGGTATTTTGATGAGGAGTTCACGGCCCAGATGATCACCATCACA
CCACCTGACCAAGATGACAGCATGGAGTGTGTGGACAGCGAGCGC
AGGCCCCACTTCCCCCAGTTCTCCTACTCGGCCAGCGGCACGGCC
TGA
The sequence of MYOD1 cDNA comprises
SEQ ID No. 16:
>ENST00000250003.4 MYOD1-201 cdna:
protein_coding
ATGGAGCTACTGTCGCCACCGCTCCGCGACGTAGACCTGACGGCC
CCCGACGGCTCTCTCTGCTCCTTTGCCACAACGGACGACTTCTAT
GACGACCCGTGTTTCGACTCCCCGGACCTGCGCTTCTTCGAAGAC
CTGGACCCGCGCCTGATGCACGTGGGCGCGCTCCTGAAACCCGAA
GAGCACTCGCACTTCCCCGCGGCGGTGCACCCGGCCCCGGGCGCA
CGTGAGGACGAGCATGTGCGCGCGCCCAGCGGGCACCACCAGGCG
GGCCGCTGCCTACTGTGGGCCTGCAAGGCGTGCAAGCGCAAGACC
ACCAACGCCGACCGCCGCAAGGCCGCCACCATGCGCGAGCGGCGC
CGCCTGAGCAAAGTAAATGAGGCCTTTGAGACACTCAAGCGCTGC
ACGTCGAGCAATCCAAACCAGCGGTTGCCCAAGGTGGAGATCCTG
CGCAACGCCATCCGCTATATCGAGGGCCTGCAGGCTCTGCTGCGC
GACCAGGACGCCGCGCCCCCTGGCGCCGCAGCCGCCTTCTATGCG
CCGGGCCCGCTGCCCCCGGGCCGCGGCGGCGAGCACTACAGCGGC
GACTCCGACGCGTCCAGCCCGCGCTCCAACTGCTCCGACGGCATG
ATGGACTACAGCGGCCCCCCGAGCGGCGCCCGGCGGCGGAACTGC
TACGAAGGCGCCTACTACAACGAGGCGCCCAGCGAACCCAGGCCC
GGGAAGAGTGCGGCGGTGTCGAGCCTAGACTGCCTGTCCAGCATC
GTGGAGCGCATCTCCACCGAGAGCCCTGCGGCGCCCGCCCTCCTG
CTGGCGGACGTGCCTTCTGAGTCGCCTCCGCGCAGGCAAGAGGCT
GCCGCCCCCAGCGAGGGAGAGAGCAGCGGCGACCCCACCCAGTCA
CCGGACGCCGCCCCGCAGTGCCCTGCGGGTGCGAACCCCAACCCG
ATATACCAGGTGCTCTGA
The sequence of ACE cDNA comprises
SEQ ID No. 17:
>ENST00000290866.10 ACE-202 cdna:
protein_coding
ATGGGGGCCGCCTCGGGCCGCCGGGGGCCGGGGCTGCTGCTGCCG
CTGCCGCTGCTGTTGCTGCTGCCGCCGCAGCCCGCCCTGGCGTTG
GACCCCGGGCTGCAGCCCGGCAACTTTTCTGCTGACGAGGCCGGG
GCGCAGCTCTTCGCGCAGAGCTACAACTCCAGCGCCGAACAGGTG
CTGTTCCAGAGCGTGGCCGCCAGCTGGGCGCACGACACCAACATC
ACCGCGGAGAATGCAAGGCGCCAGGAGGAAGCAGCCCTGCTCAGC
CAGGAGTTTGCGGAGGCCTGGGGCCAGAAGGCCAAGGAGCTGTAT
GAACCGATCTGGCAGAACTTCACGGACCCGCAGCTGCGCAGGATC
ATCGGAGCTGTGCGCACCCTGGGCTCTGCCAACCTGCCCCTGGCT
AAGCGGCAGCAGTACAACGCCCTGCTAAGCAACATGAGCAGGATC
TACTCCACCGCCAAGGTCTGCCTCCCCAACAAGACTGCCACCTGC
TGGTCCCTGGACCCAGATCTCACCAACATCCTGGCTTCCTCGCGA
AGCTACGCCATGCTCCTGTTTGCCTGGGAGGGCTGGCACAACGCT
GCGGGCATCCCGCTGAAACCGCTGTACGAGGATTTCACTGCCCTC
AGCAATGAAGCCTACAAGCAGGACGGCTTCACAGACACGGGGGCC
TACTGGCGCTCCTGGTACAACTCCCCCACCTTCGAGGACGATCTG
GAACACCTCTACCAACAGCTAGAGCCCCTCTACCTGAACCTCCAT
GCCTTCGTCCGCCGCGCACTGCATCGCCGATACGGAGACAGATAC
ATCAACCTCAGGGGACCCATCCCTGCTCATCTGCTGGGAGACATG
TGGGCCCAGAGCTGGGAAAACATCTACGACATGGTGGTGCCTTTC
CCAGACAAGCCCAACCTCGATGTCACCAGTACTATGCTGCAGCAG
GGCTGGAACGCCACGCACATGTTCCGGGTGGCAGAGGAGTTCTTC
ACCTCCCTGGAGCTCTCCCCCATGCCTCCCGAGTTCTGGGAAGGG
TCGATGCTGGAGAAGCCGGCCGACGGGCGGGAAGTGGTGTGCCAC
GCCTCGGCTTGGGACTTCTACAACAGGAAAGACTTCAGGATCAAG
CAGTGCACACGGGTCACGATGGACCAGCTCTCCACAGTGCACCAT
GAGATGGGCCATATACAGTACTACCTGCAGTACAAGGATCTGCCC
GTCTCCCTGCGTCGGGGGGCCAACCCCGGCTTCCATGAGGCCATT
GGGGACGTGCTGGCGCTCTCGGTCTCCACTCCTGAACATCTGCAC
AAAATCGGCCTGCTGGACCGTGTCACCAATGACACGGAAAGTGAC
ATCAATTACTTGCTAAAAATGGCACTGGAAAAAATTGCCTTCCTG
CCCTTTGGCTACTTGGTGGACCAGTGGCGCTGGGGGGTCTTTAGT
GGGCGTACCCCCCCTTCCCGCTACAACTTCGACTGGTGGTATCTT
CGAACCAAGTATCAGGGGATCTGTCCTCCTGTTACCCGAAACGAA
ACCCACTTTGATGCTGGAGCTAAGTTTCATGTTCCAAATGTGACA
CCATACATCAGGTACTTTGTGAGTTTTGTCCTGCAGTTCCAGTTC
CATGAAGCCCTGTGCAAGGAGGCAGGCTATGAGGGCCCACTGCAC
CAGTGTGACATCTACCGGTCCACCAAGGCAGGGGCCAAGCTCCGG
AAGGTGCTGCAGGCTGGCTCCTCCAGGCCCTGGCAGGAGGTGCTG
AAGGACATGGTCGGCTTAGATGCCCTGGATGCCCAGCCGCTGCTC
AAGTACTTCCAGCCAGTCACCCAGTGGCTGCAGGAGCAGAACCAG
CAGAACGGCGAGGTCCTGGGCTGGCCCGAGTACCAGTGGCACCCG
CCGTTGCCTGACAACTACCCGGAGGGCATAGACCTGGTGACTGAT
GAGGCTGAGGCCAGCAAGTTTGTGGAGGAATATGACCGGACATCC
CAGGTGGTGTGGAACGAGTATGCCGAGGCCAACTGGAACTACAAC
ACCAACATCACCACAGAGACCAGCAAGATTCTGCTGCAGAAGAAC
ATGCAAATAGCCAACCACACCCTGAAGTACGGCACCCAGGCCAGG
AAGTTTGATGTGAACCAGTTGCAGAACACCACTATCAAGCGGATC
ATAAAGAAGGTTCAGGACCTAGAACGGGCAGCACTGCCTGCCCAG
GAGCTGGAGGAGTACAACAAGATCCTGTTGGATATGGAAACCACC
TACAGCGTGGCCACTGTGTGCCACCCGAATGGCAGCTGCCTGCAG
CTCGAGCCAGATCTGACGAATGTGATGGCCACGTCCCGGAAATAT
GAAGACCTGTTATGGGCATGGGAGGGCTGGCGAGACAAGGCGGGG
AGAGCCATCCTCCAGTTTTACCCGAAATACGTGGAACTCATCAAC
CAGGCTGCCCGGCTCAATGGCTATGTAGATGCAGGGGACTCGTGG
AGGTCTATGTACGAGACACCATCCCTGGAGCAAGACCTGGAGCGG
CTCTTCCAGGAGCTGCAGCCACTCTACCTCAACCTGCATGCCTAC
GTGCGCCGGGCCCTGCACCGTCACTACGGGGCCCAGCACATCAAC
CTGGAGGGGCCCATTCCTGCTCACCTGCTGGGGAACATGTGGGCG
CAGACCTGGTCCAACATCTATGACTTGGTGGTGCCCTTCCCTTCA
GCCCCCTCGATGGACACCACAGAGGCTATGCTAAAGCAGGGCTGG
ACGCCCAGGAGGATGTTTAAGGAGGCTGATGATTTCTTCACCTCC
CTGGGGCTGCTGCCCGTGCCTCCTGAGTTCTGGAACAAGTCGATG
CTGGAGAAGCCAACCGACGGGCGGGAGGTGGTCTGCCACGCCTCG
GCCTGGGACTTCTACAACGGCAAGGACTTCCGGATCAAGCAGTGC
ACCACCGTGAACTTGGAGGACCTGGTGGTGGCCCACCACGAAATG
GGCCACATCCAGTATTTCATGCAGTACAAAGACTTACCTGTGGCC
TTGAGGGAGGGTGCCAACCCCGGCTTCCATGAGGCCATTGGGGAC
GTGCTAGCCCTCTCAGTGTCTACGCCCAAGCACCTGCACAGTCTC
AACCTGCTGAGCAGTGAGGGTGGCAGCGACGAGCATGACATCAAC
TTTCTGATGAAGATGGCCCTTGACAAGATCGCCTTTATCCCCTTC
AGCTACCTCGTCGATCAGTGGCGCTGGAGGGTATTTGATGGAAGC
ATCACCAAGGAGAACTATAACCAGGAGTGGTGGAGCCTCAGGCTG
AAGTACCAGGGCCTCTGCCCCCCAGTGCCCAGGACTCAAGGTGAC
TTTGACCCAGGGGCCAAGTTCCACATTCCTTCTAGCGTGCCTTAC
ATCAGGTACTTTGTCAGCTTCATCATCCAGTTCCAGTTCCACGAG
GCACTGTGCCAGGCAGCTGGCCACACGGGCCCCCTGCACAAGTGT
GACATCTACCAGTCCAAGGAGGCCGGGCAGCGCCTGGCGACCGCC
ATGAAGCTGGGCTTCAGTAGGCCGTGGCCGGAAGCCATGCAGCTG
ATCACGGGCCAGCCCAACATGAGCGCCTCGGCCATGTTGAGCTAC
TTCAAGCCGCTGCTGGACTGGCTCCGCACGGAGAACGAGCTGCAT
GGGGAGAAGCTGGGCTGGCCGCAGTACAACTGGACGCCGAACTCC
GCTCGCTCAGAAGGGCCCCTCCCAGACAGCGGCCGCGTCAGCTTC
CTGGGCCTGGACCTGGATGCGCAGCAGGCCCGCGTGGGCCAGTGG
CTGCTGCTCTTCCTGGGCATCGCCCTGCTGGTAGCCACCCTGGGC
CTCAGCCAGCGGCTCTTCAGCATCCGCCACCGCAGCCTCCACCGG
CACTCCCACGGGCCCCAGTTCGGCTCCGAGGTGGAGCTGAGACAC
TCCTGA
The sequence of PPARGCIA cDNA comprises
SEQ ID No. 18:
>ENST00000264867.7 PPARGC1A-201 cdna:
protein_coding
ATGGCGTGGGACATGTGCAACCAGGACTCTGAGTCTGTATGGAGT
GACATCGAGTGTGCTGCTCTGGTTGGTGAAGACCAGCCTCTTTGC
CCAGATCTTCCTGAACTTGATCTTTCTGAACTAGATGTGAACGAC
TTGGATACAGACAGCTTTCTGGGTGGACTCAAGTGGTGCAGTGAC
CAATCAGAAATAATATCCAATCAGTACAACAATGAGCCTTCAAAC
ATATTTGAGAAGATAGATGAAGAGAATGAGGCAAACTTGCTAGCA
GTCCTCACAGAGACACTAGACAGTCTCCCTGTGGATGAAGACGGA
TTGCCCTCATTTGATGCGCTGACAGATGGAGACGTGACCACTGAC
AATGAGGCTAGTCCTTCCTCCATGCCTGACGGCACCCCTCCACCC
CAGGAGGCAGAAGAGCCGTCTCTACTTAAGAAGCTCTTACTGGCA
CCAGCCAACACTCAGCTAAGTTATAATGAATGCAGTGGTCTCAGT
ACCCAGAACCATGCAAATCACAATCACAGGATCAGAACAAACCCT
GCAATTGTTAAGACTGAGAATTCATGGAGCAATAAAGCGAAGAGT
ATTTGTCAACAGCAAAAGCCACAAAGACGTCCCTGCTCGGAGCTT
CTCAAATATCTGACCACAAACGATGACCCTCCTCACACCAAACCC
ACAGAGAACAGAAACAGCAGCAGAGACAAATGCACCTCCAAAAAG
AAGTCCCACACACAGTCGCAGTCACAACACTTACAAGCCAAACCA
ACAACTTTATCTCTTCCTCTGACCCCAGAGTCACCAAATGACCCC
AAGGGTTCCCCATTTGAGAACAAGACTATTGAACGCACCTTAAGT
GTGGAACTCTCTGGAACTGCAGGCCTAACTCCACCCACCACTCCT
CCTCATAAAGCCAACCAAGATAACCCTTTTAGGGCTTCTCCAAAG
CTGAAGTCCTCTTGCAAGACTGTGGTGCCACCACCATCAAAGAAG
CCCAGGTACAGTGAGTCTTCTGGTACACAAGGCAATAACTCCACC
AAGAAAGGGCCGGAGCAATCCGAGTTGTATGCACAACTCAGCAAG
TCCTCAGTCCTCACTGGTGGACACGAGGAAAGGAAGACCAAGCGG
CCCAGTCTGCGGCTGTTTGGTGACCATGACTATTGCCAGTCAATT
AATTCCAAAACAGAAATACTCATTAATATATCACAGGAGCTCCAA
GACTCTAGACAACTAGAAAATAAAGATGTCTCCTCTGATTGGCAG
GGGCAGATTTGTTCTTCCACAGATTCAGACCAGTGCTACCTGAGA
GAGACTTTGGAGGCAAGCAAGCAGGTCTCTCCTTGCAGCACAAGA
AAACAGCTCCAAGACCAGGAAATCCGAGCCGAGCTGAACAAGCAC
TTCGGTCATCCCAGTCAAGCTGTTTTTGACGACGAAGCAGACAAG
ACCGGTGAACTGAGGGACAGTGATTTCAGTAATGAACAATTCTCC
AAACTACCTATGTTTATAAATTCAGGACTAGCCATGGATGGCCTG
TTTGATGACAGCGAAGATGAAAGTGATAAACTGAGCTACCCTTGG
GATGGCACGCAATCCTATTCATTGTTCAATGTGTCTCCTTCTTGT
TCTTCTTTTAACTCTCCATGTAGAGATTCTGTGTCACCACCCAAA
TCCTTATTTTCTCAAAGACCCCAAAGGATGCGCTCTCGTTCAAGG
TCCTTTTCTCGACACAGGTCGTGTTCCCGATCACCATATTCCAGG
TCAAGATCAAGGTCTCCAGGCAGTAGATCCTCTTCAAGATCCTGC
TATTACTATGAGTCAAGCCACTACAGACACCGCACGCACCGAAAT
TCTCCCTTGTATGTGAGATCACGTTCAAGATCGCCCTACAGCCGT
CGGCCCAGGTATGACAGCTACGAGGAATATCAGCACGAGAGGCTG
AAGAGGGAAGAATATCGCAGAGAGTATGAGAAGCGAGAGTCTGAG
AGGGCCAAGCAAAGGGAGAGGCAGAGGCAGAAGGCAATTGAAGAG
CGCCGTGTGATTTATGTCGGTAAAATCAGACCTGACACAACACGG
ACAGAACTGAGGGACCGTTTTGAAGTTTTTGGTGAAATTGAGGAG
TGCACAGTAAATCTGCGGGATGATGGAGACAGCTATGGTTTCATT
ACCTACCGTTATACCTGTGATGCTTTTGCTGCTCTTGAAAATGGA
TACACTTTGCGCAGGTCAAACGAAACTGACTTTGAGCTGTACTTT
TGTGGACGCAAGCAATTTTTCAAGTCTAACTATGCAGACCTAGAT
TCAAACTCAGATGACTTTGACCCTGCTTCCACCAAGAGCAAGTAT
GACTCTCTGGATTTTGATAGTTTACTGAAAGAAGCTCAGAGAAGC
TTGCGCAGGTAA
The sequence of Elov15 cDNA comprises
SEQ ID No. 19:
>ENST00000304434.11 ELOVL5-201 cdna:
protein_coding
ATGGAACATTTTGATGCATCACTTAGTACCTATTTCAAGGCATTG
CTAGGCCCTCGAGATACTAGAGTAAAAGGATGGTTTCTTCTGGAC
AATTATATACCCACATTTATCTGCTCTGTCATATATTTACTAATT
GTATGGCTGGGACCAAAATACATGAGGAATAAACAGCCATTCTCT
TGCCGGGGGATTTTAGTGGTGTATAACCTTGGACTCACACTGCTG
TCTCTGTATATGTTCTGTGAGTTAGTAACAGGAGTATGGGAAGGC
AAATACAACTTCTTCTGTCAGGGCACACGCACCGCAGGAGAATCA
GATATGAAGATTATCCGTGTCCTCTGGTGGTACTACTTCTCCAAA
CTCATAGAATTTATGGACACTTTCTTCTTCATCCTGCGCAAGAAC
AACCACCAGATCACGGTCCTGCACGTCTACCACCATGCCTCGATG
CTGAACATCTGGTGGTTTGTGATGAACTGGGTCCCCTGCGGCCAC
TCTTATTTTGGTGCCACACTTAATAGCTTCATCCACGTCCTCATG
TACTCTTACTATGGTTTGTCGTCAGTCCCTTCCATGCGTCCATAC
CTCTGGTGGAAGAAGTACATCACTCAGGGGCAGCTGCTTCAGTTT
GTGCTGACAATCATCCAGACCAGCTGCGGGGTCATCTGGCCGTGC
ACATTCCCTCTTGGTTGGTTGTATTTCCAGATTGGATACATGATT
TCCCTGATTGCTCTCTTCACAAACTTCTACATTCAGACCTACAAC
AAGAAAGGGGCCTCCCGAAGGAAAGACCACCTGAAGGACCACCAG
AATGGGTCCATGGCTGCTGTGAATGGACACACCAACAGCTTTTCA
CCCCTGGAAAACAATGTGAAGCCAAGGAAGCTGCGGAAGGATTGA
The sequence of Slc2a4 cDNA comprises
SEQ ID No. 20:
>ENST00000317370.13 SLC2A4-201 cdna:
protein_coding
ATGCCGTCGGGCTTCCAACAGATAGGCTCCGAAGATGGGGAACCC
CCTCAGCAGCGAGTGACTGGGACCCTGGTCCTTGCTGTGTTCTCT
GCGGTGCTTGGCTCCCTGCAGTTTGGGTACAACATTGGGGTCATC
AATGCCCCTCAGAAGGTGATTGAACAGAGCTACAATGAGACGTGG
CTGGGGAGGCAGGGGCCTGAGGGACCCAGCTCCATCCCTCCAGGC
ACCCTCACCACCCTCTGGGCCCTCTCCGTGGCCATCTTTTCCGTG
GGCGGCATGATTTCCTCCTTCCTCATTGGTATCATCTCTCAGTGG
CTTGGAAGGAAAAGGGCCATGCTGGTCAACAATGTCCTGGCGGTG
CTGGGGGGCAGCCTCATGGGCCTGGCCAATGCTGCTGCCTCCTAT
GAAATGCTCATCCTTGGACGATTCCTCATTGGCGCCTACTCAGGG
CTGACATCAGGGCTGGTGCCCATGTACGTGGGGGAGATTGCTCCC
ACTCACCTGCGGGGCGCCCTGGGGACGCTCAACCAACTGGCCATT
GTTATCGGCATTCTGATCGCCCAGGTGCTGGGCTTGGAGTCCCTC
CTGGGCACTGCCAGCCTGTGGCCACTGCTCCTGGGCCTCACAGTG
CTACCTGCCCTCCTGCAGCTGGTCCTGCTGCCCTTCTGTCCCGAG
AGCCCCCGCTACCTCTACATCATCCAGAATCTCGAGGGGCCTGCC
AGAAAGAGTCTGAAGCGCCTGACAGGCTGGGCCGATGTTTCTGGA
GTGCTGGCTGAGCTGAAGGATGAGAAGCGGAAGCTGGAGCGTGAG
CGGCCACTGTCCCTGCTCCAGCTCCTGGGCAGCCGTACCCACCGG
CAGCCCCTGATCATTGCGGTCGTGCTGCAGCTGAGCCAGCAGCTC
TCTGGCATCAATGCTGTTTTCTATTATTCGACCAGCATCTTCGAG
ACAGCAGGGGTAGGCCAGCCTGCCTATGCCACCATAGGAGCTGGT
GTGGTCAACACAGTCTTCACCTTGGTCTCGGTGTTGTTGGTGGAG
CGGGCGGGGCGCCGGACGCTCCATCTCCTGGGCCTGGCGGGCATG
TGTGGCTGTGCCATCCTGATGACTGTGGCTCTGCTCCTGCTGGAG
CGAGTTCCAGCCATGAGCTACGTCTCCATTGTGGCCATCTTTGGC
TTCGTGGCATTTTTTGAGATTGGCCCTGGCCCCATTCCTTGGTTC
ATCGTGGCCGAGCTCTTCAGCCAGGGACCCCGCCCGGCAGCCATG
GCTGTGGCTGGTTTCTCCAACTGGACGAGCAACTTCATCATTGGC
ATGGGTTTCCAGTATGTTGCGGAGGCTATGGGGCCCTACGTCTTC
CTTCTATTTGCGGTCCTCCTGCTGGGCTTCTTCATCTTCACCTTC
TTAAGAGTACCTGAAACTCGAGGCCGGACGTTTGACCAGATCTCA
GCTGCCTTCCACCGGACACCCTCTCTTTTAGAGCAGGAGGTGAAA
CCCAGCACAGAACTTGAGTATTTAGGGCCAGATGAGAACGACTGA

In one embodiment of the method according to the invention samples of at least two consecutive days of said subject are provided and the amount of gene expression is determined and used for said assessment and/or prediction, preferably at least three samples per day, more preferably at least four samples per day.

Subject matter of the present invention is a method of predicting the individual diurnal athletic performance time(s) of a subject, wherein each of the time points at which said samples are obtained are at least 2-4 hours apart, and/or wherein the time points span a time period of at least 12 hours of the day, wherein preferably the time points are 4 hours apart, e.g. at 9 h, 13 h, 17 h and 21 h. The specific times can be chosen based on the individual wake up time. For e.g. for someone who usually wakes up at 11 h one would start at 11 h.

Subject matter of the present invention is a kit for sampling saliva for use in a method according to the present invention comprising:
sampling tubes for receiving the samples of saliva, wherein each of the sampling tubes contains RNA protect reagent and is configured to enclose one of the samples of saliva to be taken together with the reagent,
wherein preferably each of the sampling tubes is labelled with the time point at which the respective sample is to be taken and/or includes an indication about the amount of saliva to be taken for one sample,

The kit may further comprise at least one of a box, a cool pack, at least one form including instructions and/or information about the kit and the method for the subject.

RNA protect agents are known in the art and may be selected from the group comprising EDTA disodium, dihydrate; sodium citrate trisodium salt, dihydrate; ammonium sulfate, powdered; sterile water, wherein a single reagent or a combination of different reagents may be used.

In one embodiment of the kit said sampling tubes are configured to receive a sample of saliva of 1 mL in addition to 1 mL of the RNA protect reagent. The sampling tubes may be at least 2 mL tubes, preferably at least 3 mL tubes, more preferably at least 4 mL tubes, still preferably at least 5 mL tubes. While the size for the tubes of 2 mL would be sufficient, it may be more convenient for collecting the saliva samples if the tubes are bigger, such as e.g. 5 mL tubes. In order to collect the required number of samples, the kit may at least six sampling tubes, preferably at least eight sampling tubes (i.e. at least three and four, respectively, samples for two days).

While the kit is used for collecting the samples, it may be designed to be used also for storage and transport of the samples. For this purpose, it is advantageous to have a cool pack in the kit. For instance, if someone is outside and needs to collect the samples, the samples could be stored at room temperature anyway for a few hours, or if one know that there will be no fridge for the next two days, one could still freeze the cool pack before the sampling, then place the cool pack in the box and sample as needed, since the box would remain cold for several hours (maybe even for two days, depending on the outside temperature). After the sampling is completed, the same box can be used to send the samples back to a lab, if applicable with all the forms inside as well (it may be required to pack the box in a post box for sending, which however may be enough for preparing the kit to be sent).

### DETAILED DESCRIPTION OF THE INVENTION

Exemplary embodiments of the present invention will be explained by way of example with reference to the drawings. In the drawings:

FIG. 1 illustrates the circadian core-clock network;

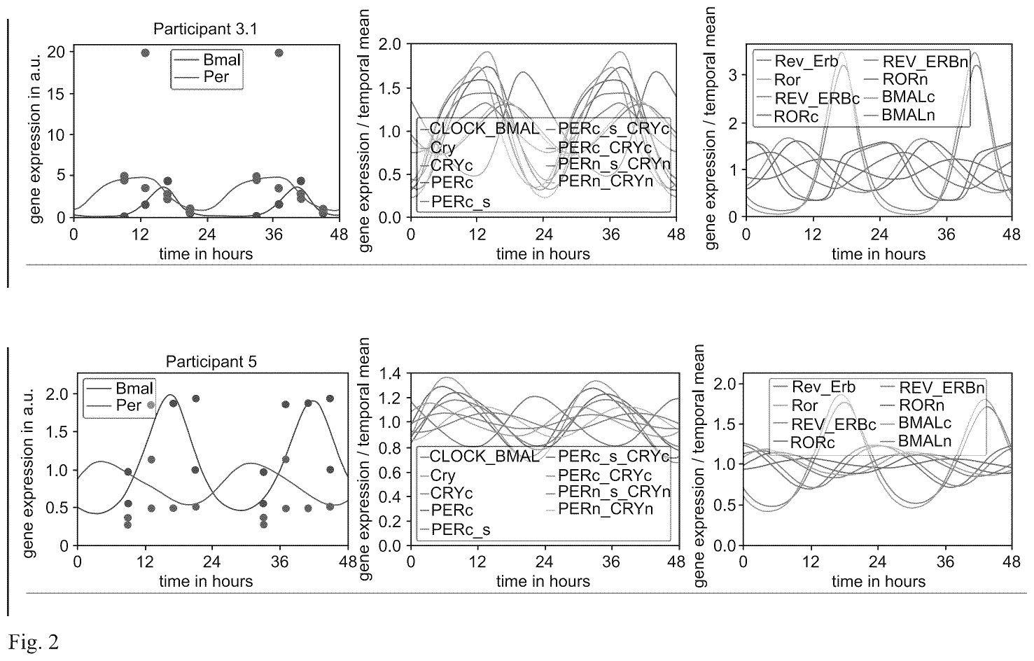

FIG. 2 illustrates two examples of fits of saliva data to a core-clock mathematical model;

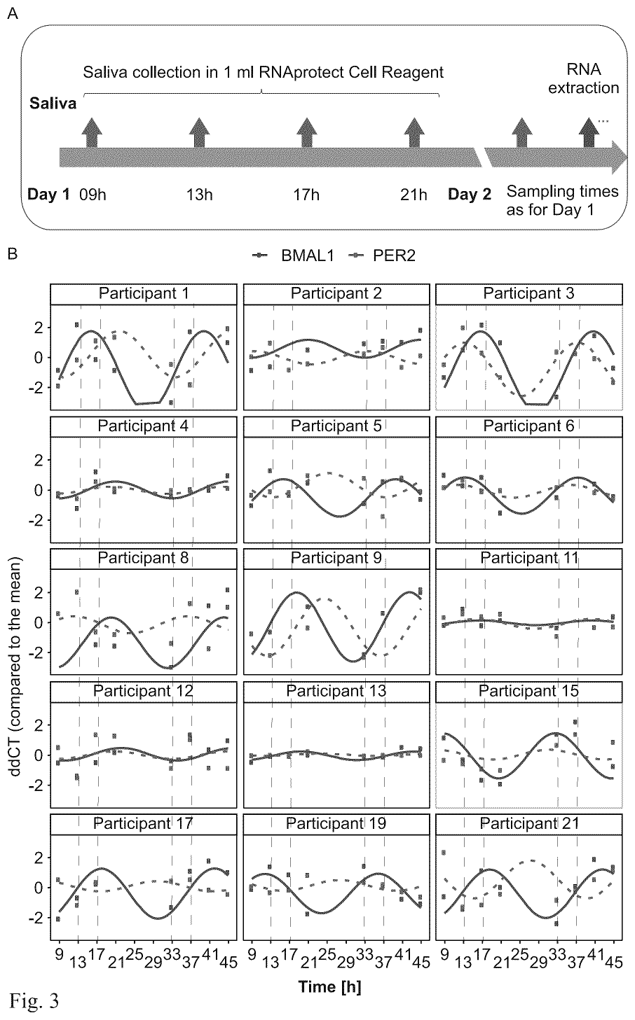

FIG. 3 illustrates time-course measurements of unstimulated saliva show fluctuations in gene expression across 45 hours for two core-clock genes (Bmal1 and PER2) as an example;

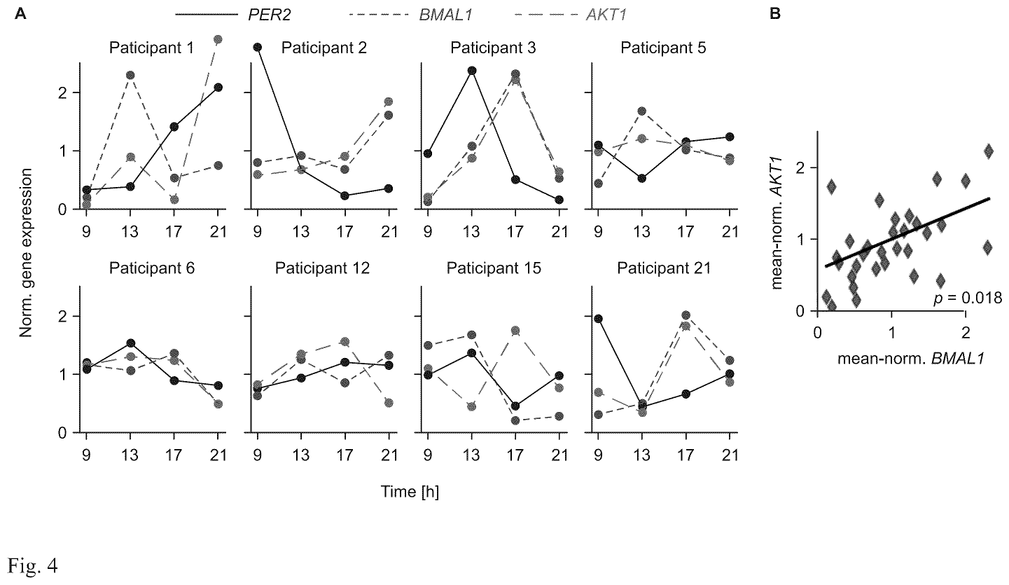

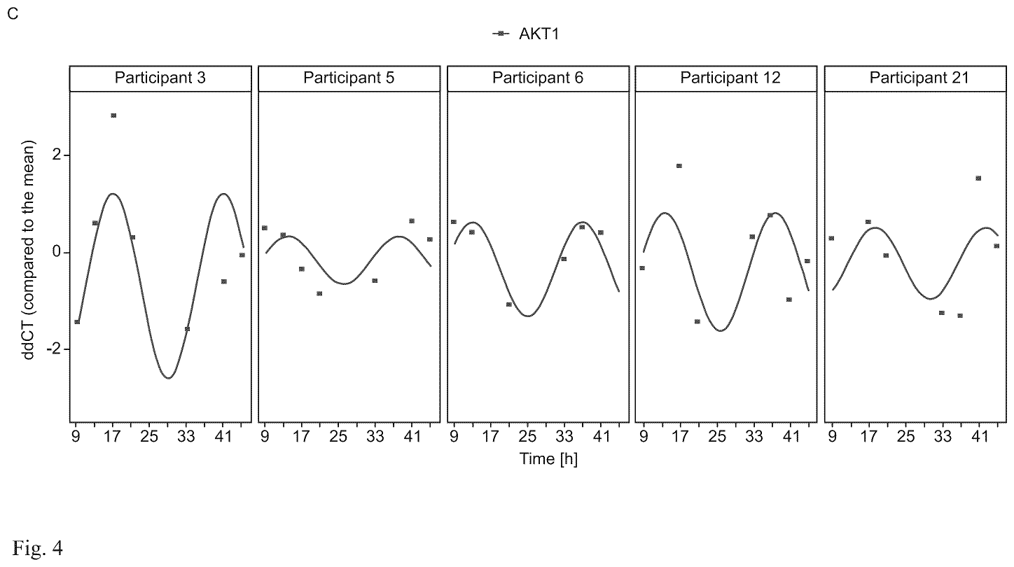

FIG. 4 illustrates how gene expression of Arntl (Bmal1) and Aktl covary. Furthermore, it is depicted that Arntl (Bmal1), Per2 and AKT1 vary in time for the different participants (A). It further shows that the variations in Akt could be correlated with variation in one of the clock genes-Bmal1 (B). It shows as well that circadian variation in Akt could be measured for the exemplified participants in the saliva.

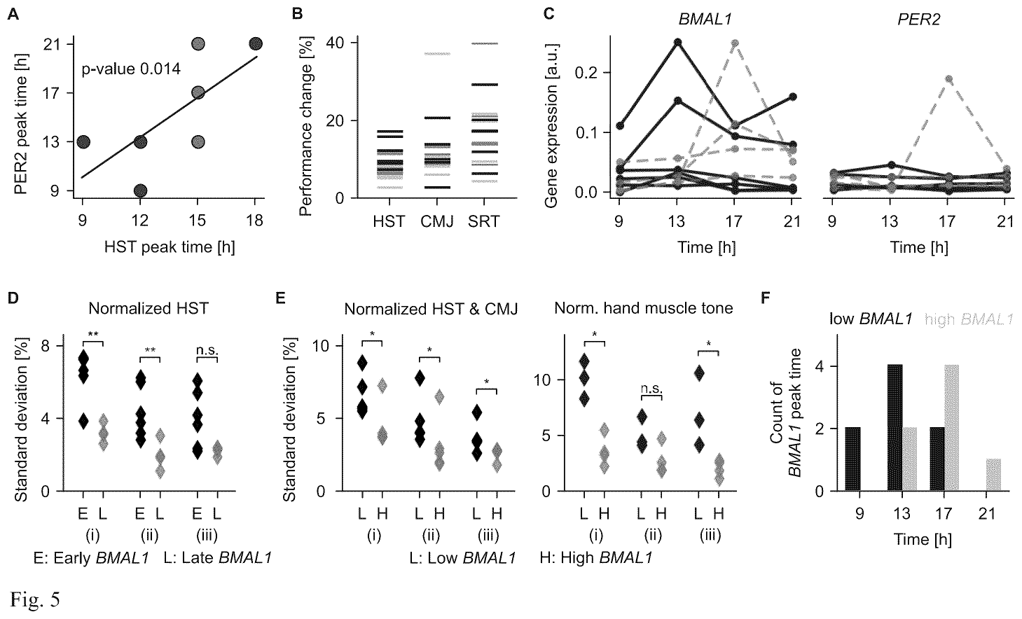

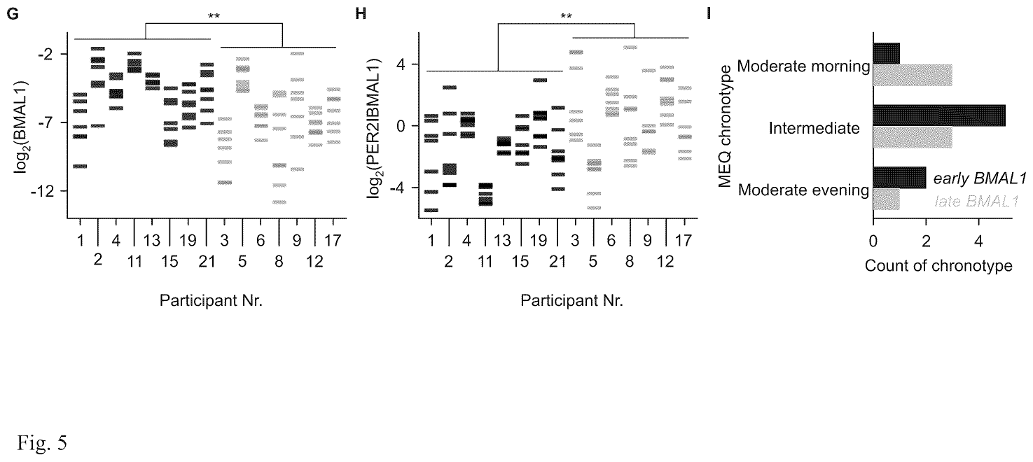

FIG. 5 illustrates correlations between molecular rhythms of core-clock genes and athletic performance; (A) The peak time of PER2 correlates with the time of peak performance of the HST (linear regression with p=0.014). (B) Performance change over the day (max. compared to min.), colour code as in (C). (C) Black and grey groups have an early and late ARNTL (BMAL1) peak time, respectively. (D) Standard deviation calculated on the normalized HST performance for data from different (i) repetitions and timepoints (p=0.0095), (ii) timepoints (p=0.0095), (iii) repetitions (p=0.057). (E) Separating the groups by the mean expression level of ARNTL (BMAL1) instead of the peak time results in significant differences in the standard deviation of the sports performance of HST and CMJ (left, all p=0.0476) and of the hand muscle frequency (right, p=0.0286, p=0.11, p=0.0286). (F) Histogram of the time of the day with the highest ARNTL (BMAL1) expression based on the eight saliva samples. Significantly earlier peaks are found for the group with low ARNTL (BMAL1) expression (ranksum, p=0.044). (G) Logarithm of ARNTL (BMAL1) expression levels for all sampling times ordered by male and female participants. Males show a significant higher ARNTL (BMAL1) expression compared to females (Welch's t-test, p<0.0001). (H) Logarithm of the ratio of PER2 and ARNTL (BMAL1) expression levels for all sampling times ordered by male and female participants. Females show a significant higher ARNTL (BMAL1) expression compared to males (Welch's t-test, p<0.0001). (I) Early or late ARNTL (BMAL1) peaks occur in any of the three investigated MEQ chronotype.

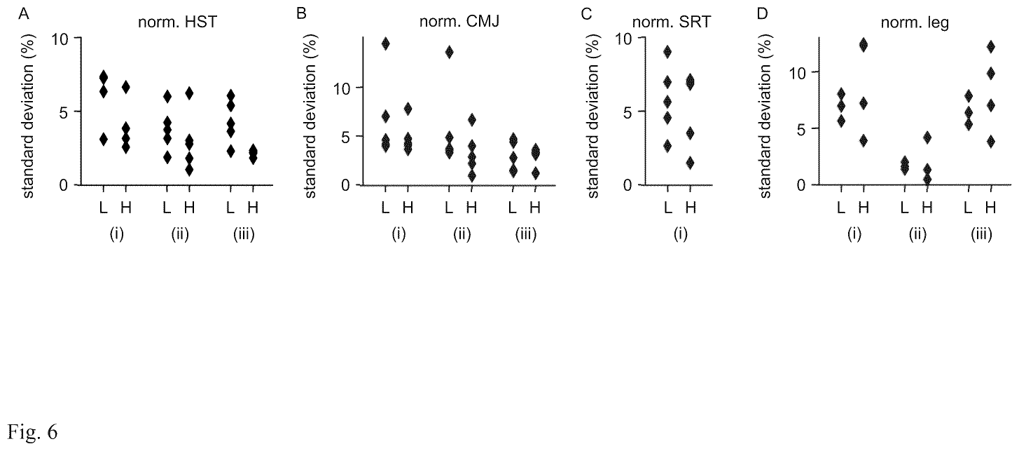

FIG. 6 illustrates standard deviations of normalized sports and muscle tone data; Standard deviations of normalized sports and muscle tone data (L: group with low BMAL1, H: group with high BMAL1). Mean standard deviation calculated on the normalized sports performance and the normalized muscle tone data for different (i) repetitions and timepoints, (ii) timepoints, (iii) repetitions (for details see Methods). (A) HST, (B) CMJ, (C) SRT (no repetitions were measured, thus the standard deviation (i) over all data is the same as (ii) over timepoints), (D) muscle tone of the leg muscles (M. rectus femoris, M. biceps femoris, M. gastrocnemius).

FIG. 7 illustrates a mathematical model extension, in which the core clock genetic network is complemented with additional genes associated to metabolism and sports performance and provides as an output performance variation in time in a personalized manner;

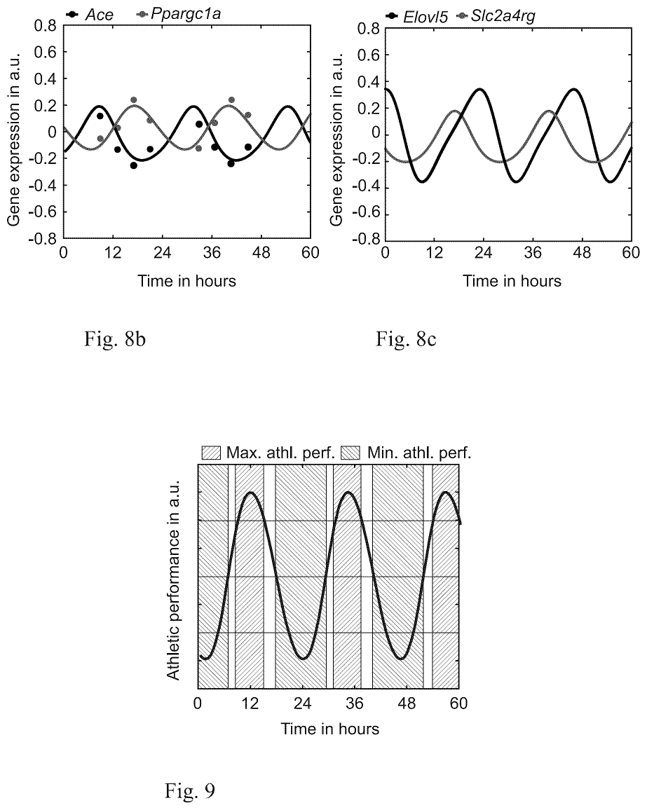

FIG. 8 illustrates an example for a personalized model fit for the core-clock genes (a) and genes important for athletic performance and metabolism (b and c) based on the expression experimental data;

FIG. 9 illustrates the computed prediction result for the athletic performance based on the expression values from FIG. 8.

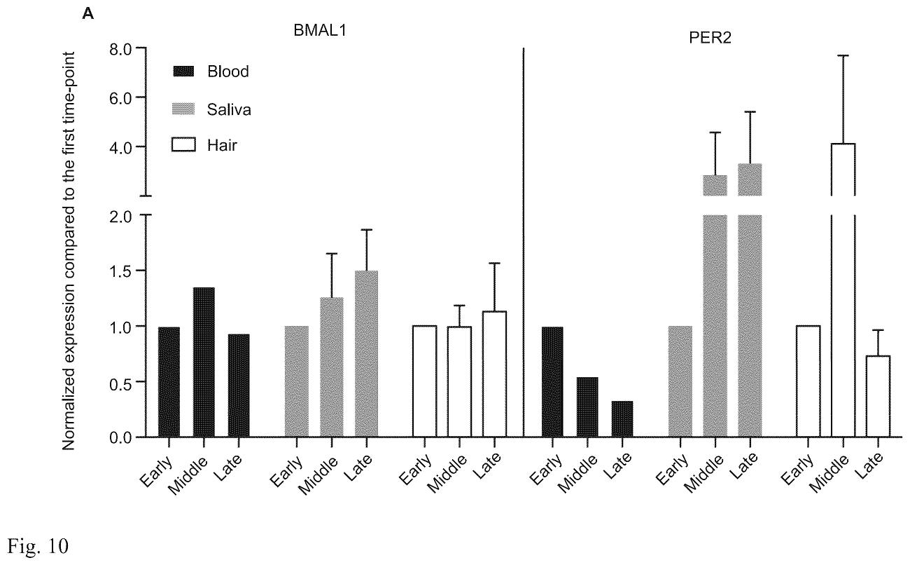

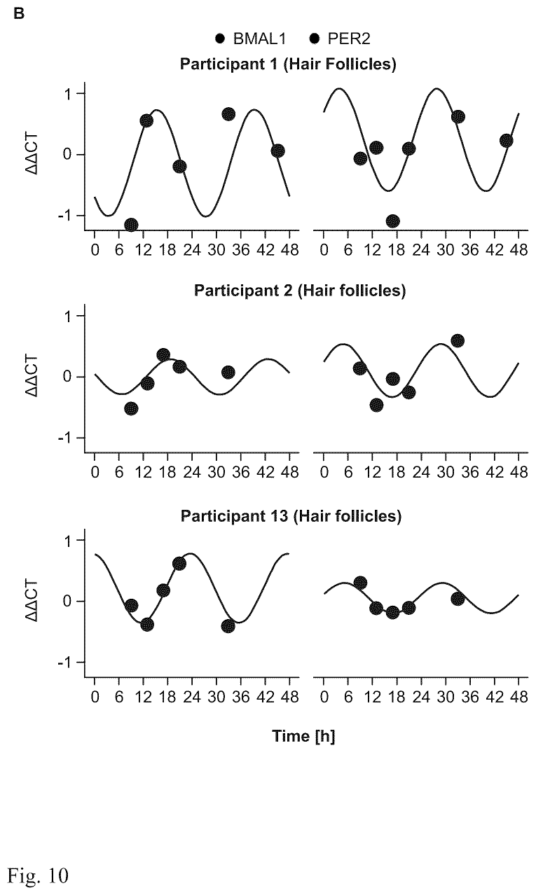

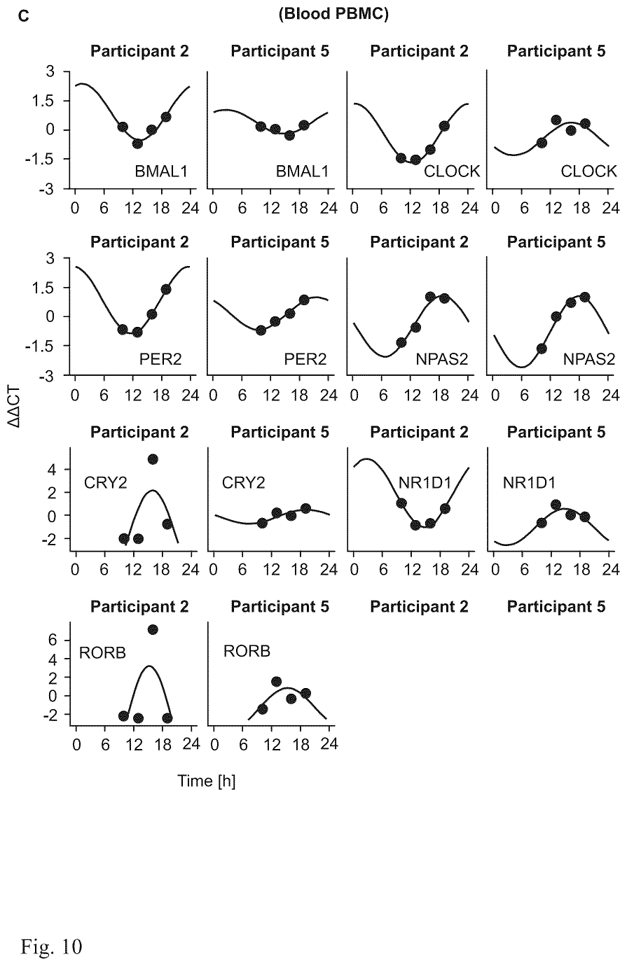

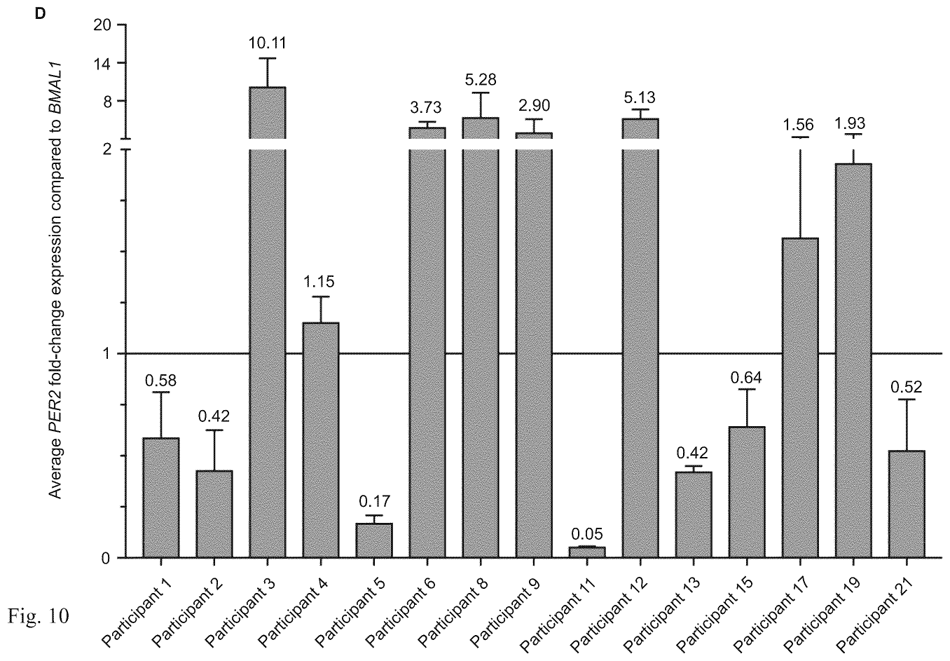

FIG. 10 illustrates ARNTL (BMAL1) and PER2 expression display variation during the day in human blood, hair and saliva samples. (A) Three time-point comparison of ARNTL (BMAL1) and PER2 expression for the averaged data of all Participants in FIG. 1. Expression data is compared to the first time-point (Early). For hair and saliva data Early, Middle and Late time-points represent 9 h, 17 h and 21 h, respectively. For PBMCs data Early, Middle and Late time-points represent 10 h, 16 h and 19 h, respectively. Depicted are mean+SEM. (B) Time-course RT-qPCR measurements normalised to the mean of all time points (ΔΔCT) of ARNTL (BMAL1) and PER2 of Participant 1, 2, and 13 with a fitted linear sine-cosine function (period=24 h). For Participant 1, we collected one additional sample at 21 h on the 2nd day. Harmonic regression best p-values for tested periods (20-28 h): Participant 1; ARNTL (BMAL1) (0.517, period=21.4 h), PER2 (0.353, period=24.0 h). Participant 2; ARNTL (BMAL1) (0.038, period=20.0 h), PER2 (0.276, period=28.0 h). Participant 13; ARNTL (BMAL1) (0.014, period=20 h), PER2 (0.086, period=21.4 h). (C) Time-course RT-qPCR measurements of human PBMCs normalised to the mean of all time points (ΔΔCT) of BMAL1 CLOCK, NPAS2, PER2, CRY2, NR1D1 and RORB of Participant 2 and 5 with a fitted linear sine-cosine function (period=24 h). Harmonic regression best p-values: Participant 2; BMAL1 (3.05 E-01, period=20 h), CLOCK (6.31 E-02, period=28 h), NPAS2 (1.67 E-01, period=20 h), PER2 (4.78 E-04, period=20.8 h), CRY2 (7.17 E-01, period=20 h), NR1D1 (1.48 E-01, period=28 h) and RORB (7.58 E-01, period=20 h). Participant 5; BMAL1 (5.56 E-01, period=20 h), CLOCK (6.81 E-01, period=28 h), NPAS2 (9.75 E-02, period=28 h, PER2 (1.23 E-01, period=28 h), CRY2 (5.40 E-01, period=28 h), NR1D1 (6.43 E-01, period=28 h) and RORB (7.73 E-01, period=28 h). (D) Average PER2 expression compared to BMAL1 using saliva time-course data for each participant (mean+SEM).

FIG. 11 illustrates HST base line measurements.

FIG. 12 illustrates Myotonometric analysis shows daily variation in muscle tone (frequency, F) for female and male participants. Only participants who completed all training sessions were included in the MyotonPRO measurements (N=12). Mean of normalized scores for the myotonometric parameter frequency [Hz] for each training session (T1-9 h, T2-12 h, T3-15 h, T4-18 h) and each muscle: M. Deltoideus, M. Triceps Brachii, M. adductor pollicis, M. rectus femoris, M. biceps femoris, M. gastrocnemius. The measurements were carried out from top to bottom on the right (Right bar) and the corresponding left (Left bar) side of the body.

FIG. 13 illustrates saliva RNA extraction optimization results. Saliva was collected from several healthy participants at 1 pm with different ratios between saliva and RNA protect reagent. Following ratios were used: 1) 1:1 with 1.5 mL saliva; 2) 1:2 with 1.0 mL saliva; 3) 2:1 with 1.0 mL saliva; 4) 1:2 with 0.5 mL saliva. Subsequently, RNA was extracted and RNA concentration was measured. Best RNA yield was achieved by using a 1:1 ratio with 1.5 mL saliva for the majority of participants.

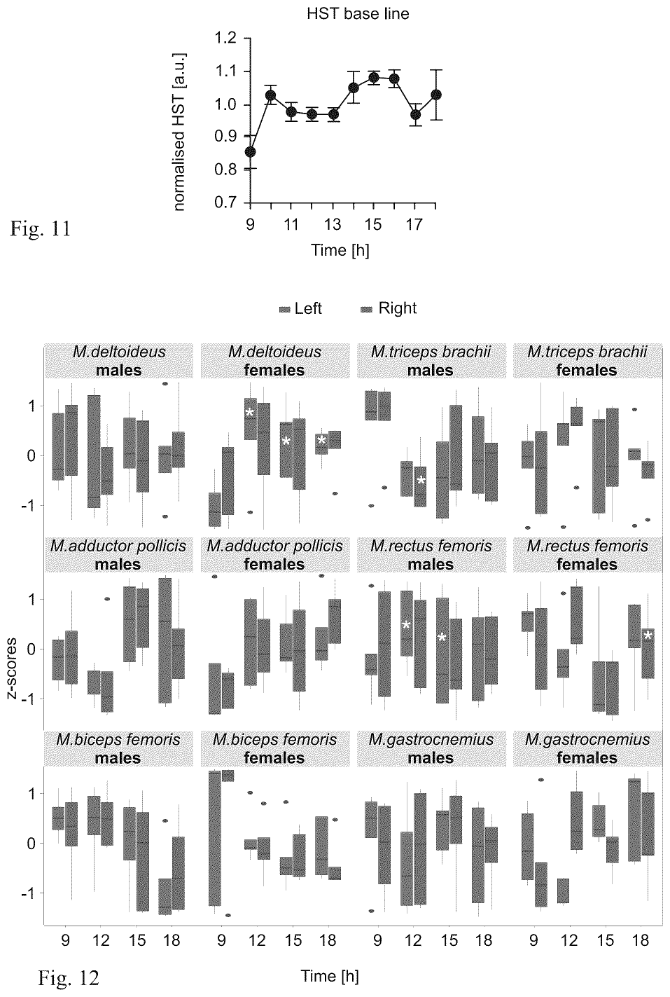

FIG. 14 illustrates time-course saliva RNA concentration results from healthy participants. Using a 1:1 ratio between saliva and RNAprotect reagent, 1.5 mL saliva was collected at several time-points per day for two consecutive days in two healthy participants, followed by RNA extraction and saliva RNA concentration measurement. In both participants and at all time-pints, saliva RNA concentration was above the minimum of 20 ng/μL, which is required for subsequent RT-PCT analysis for at least four genes.

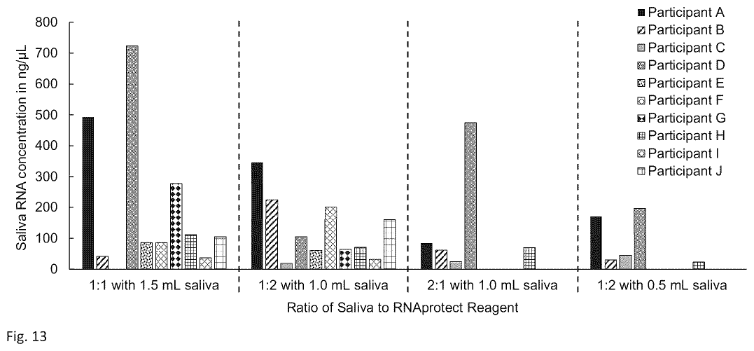

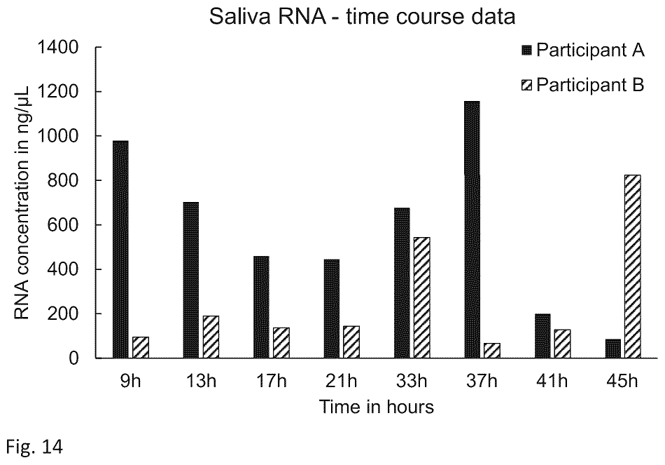

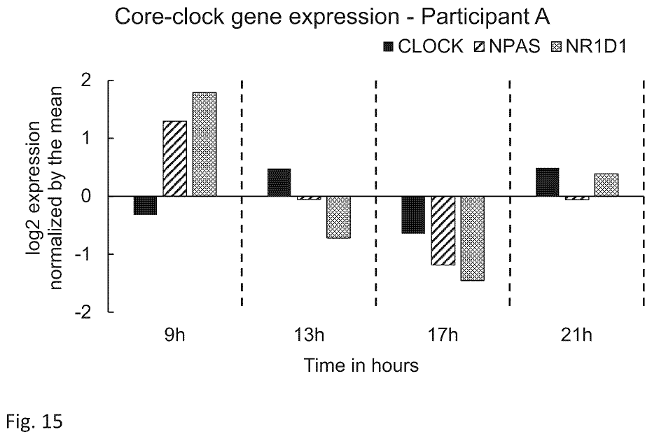

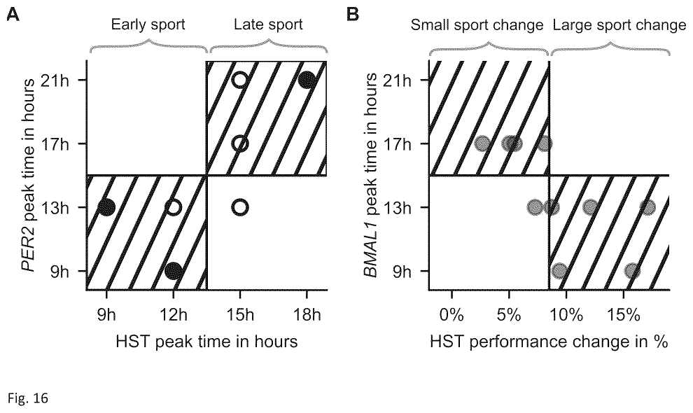

FIG. 15 illustrates time-course core-clock gene expression using saliva in healthy participant. From participant A, saliva was collected at several time-point per day (9 h, 13 h, 17 h and 21 h) using a 1:1 ratio with 1.5 mL saliva. Subsequently, RNA was extracted followed by RT-PCR detecting core-clock genes CLOCK, NPAS and NR1D1. The results show variations in the expression of core-clock genes throughout the day.

FIG. 16 illustrates predictions of exercise-related measures based on molecular rhythms of core-clock genes. (A) The peak expression of PER2 plotted against the peak performance of the hand-strength test (HST) (circles). The peak expression time of PER2 can be used to predict whether the HST performance peak is early (9 h or 12 h) or late (15 h or 18 h). Using an early PER2 peak to predict an early HST performance peak, and a late PER2 peak to predict a late HST performance peak, results in 5 correctly classified subjects with early HST performance peak (lower left quadrant, shaded), 4 correctly classified subjects with late HST performance peak (upper right quadrant, shaded), and one subject with late HST performance peak classified wrongly as early (lower right quadrant). Filled black circles represent two participants with overlapping times, not-filled black circles represent one participant. (B) The peak expression of BMAL1 plotted against the diurnal change in exercise performance of the hand-strength test (HST) for ten participants (grey filled circles). Using the peak expression time of BMAL1 to predict whether the change in HST performance is large (top five participants) or small (lower five participants) results in five correctly classified participants with large changes (lower right quadrant, shaded), and four correctly classified participants with small changes (upper left quadrant, shaded), and one participant with small changes in HST performance classified wrongly as large change (lower left quadrant).

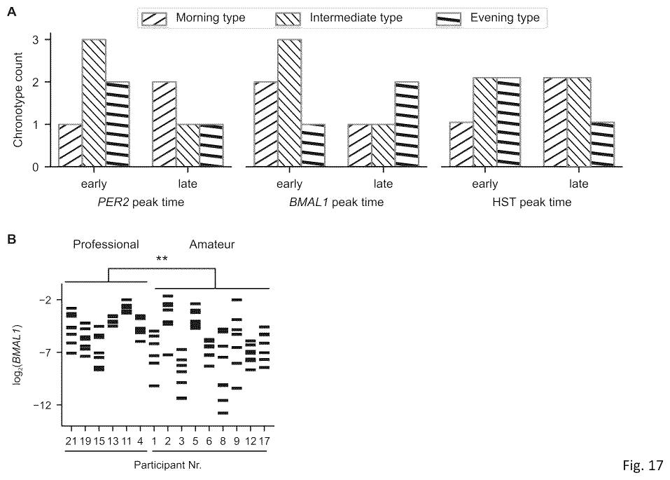

FIG. 17 illustrates the effects of chronotype and professionalism. (A) For the group of ten participants with sports data and genetic data, the chronotype distributions based on the Morningness/Eveningness Questionnaire are comparable for the subgroups with early versus late peak time for PER2, BMAL1 and HST, respectively. (B) For each participant with genetic data, the expression values of BMAL1 are plotted for all timepoints in one column. Participants with a professional background (on the left, numbers 21, 19, 15, 13, 11, 4) have a significantly higher BMAL1 expression compared to participants without a professional background (amateurs, on the right, numbers 1, 2, 3, 5, 6, 8, 9, 12, 17) (Welch's t-test, p<0.0001).

FIG. 18 illustrates an example of a physical performance prediction. (A) The subject provides saliva samples, sleep times (dashed background) and meal times (dotted vertical lines) over two days. From the saliva samples, gene expression profiles are extracted, here BMAL1 (dots), PER2 (squares) and AKT1 (diamonds). A harmonic regression curve for BMAL1 (full line), PER2 (dashed-dotted line) and AKT1 (dashed line) is shown for visualization of the genetic peak times. (B) The genetic peak time of PER2 is used to predict optimal times for exercise performance.

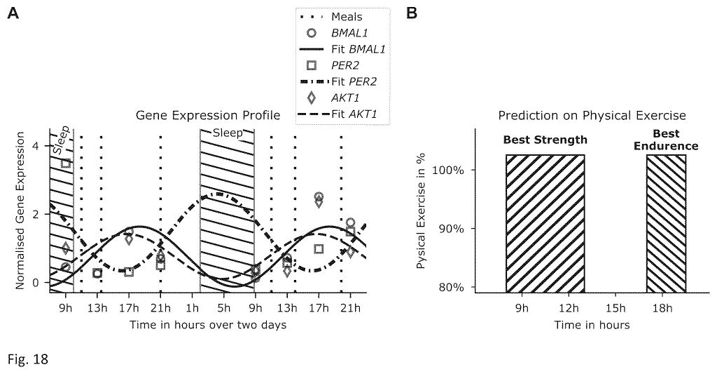

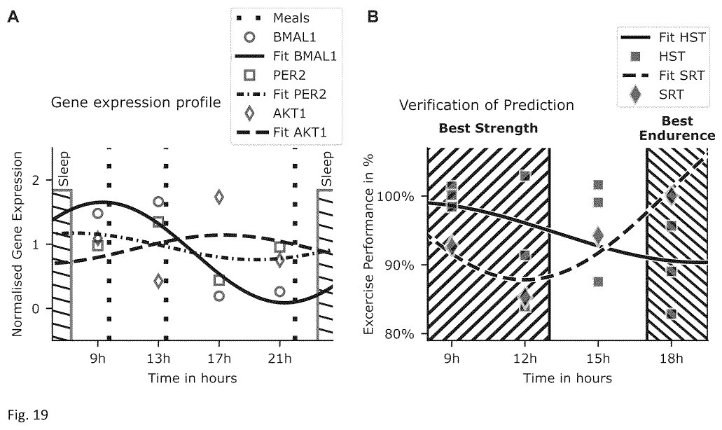

FIG. 19 illustrates an example of a physical performance prediction with a verification. (A) The subject provides saliva samples, sleep times (dashed background) and meal times (dotted vertical lines) over two days. From the saliva samples, gene expression profiles are extracted, here BMAL1 (dots), PER2 (squares) and AKT1 (diamonds). A harmonic regression curve for BMAL1 (full line), PER2 (dashed-dotted line) and AKT1 (dashed line) is shown for visualization of the genetic peak times. (B) The genetic peak time of PER2 is used to predict optimal times for exercise performance. The prediction fits with the exercise performance tested in the hand-strength test (HST, squares) and the shuttle-run test (SRT, diamonds) of this participant. For visualization, the data is fitted with a harmonic regression, HST full line, and SRT dashed line.

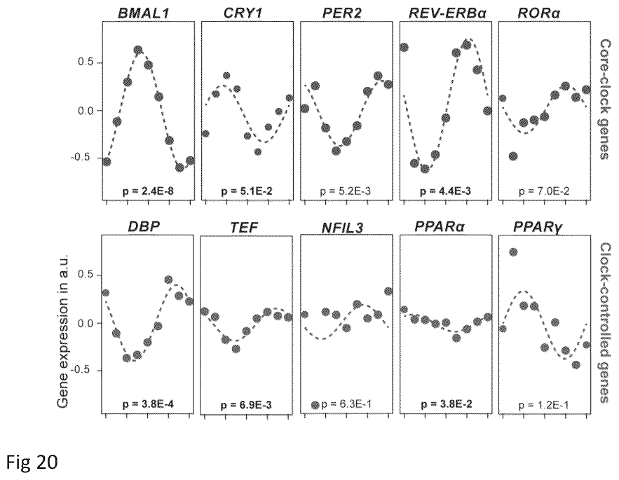

FIG. 20 illustrates a 24 h-period harmonic regression for experimental data from SW480 cell lines;

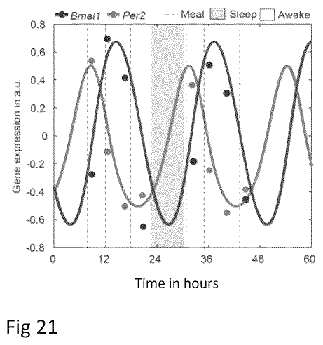

FIG. 21 illustrates an example for a personalized model fit of core-clock genes based on the experimental data. The personalized times for the particular individual (meal timing, sleep and sleep/awake times are marked for better interpretation of the results);

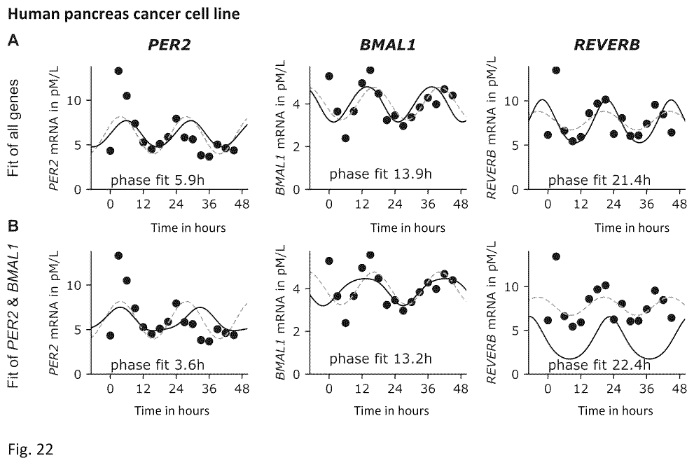

FIG. 22 illustrates a fit of the network model to a pancreas cancer cell line derived from a patient (ASPC1). (A) 48 hours time-course of gene expression for PER2, BMAL1 and REV-ERBα for ASPC1 cell line measured via RT-qPCR, multiplied by the Liver concentration of GAPDH for consistent units (dots). The harmonic regression of the data (dashed line) resembles the fit by the mammalian network model (straight line). (B) Restricting the fit to only PER2 and BMAL1, the phase for REV-ERBα is predicted with only one hour of error compared to the phase derived by also fitting REV-ERBα. Harmonic regression (dashed line) and model fit (straight line).

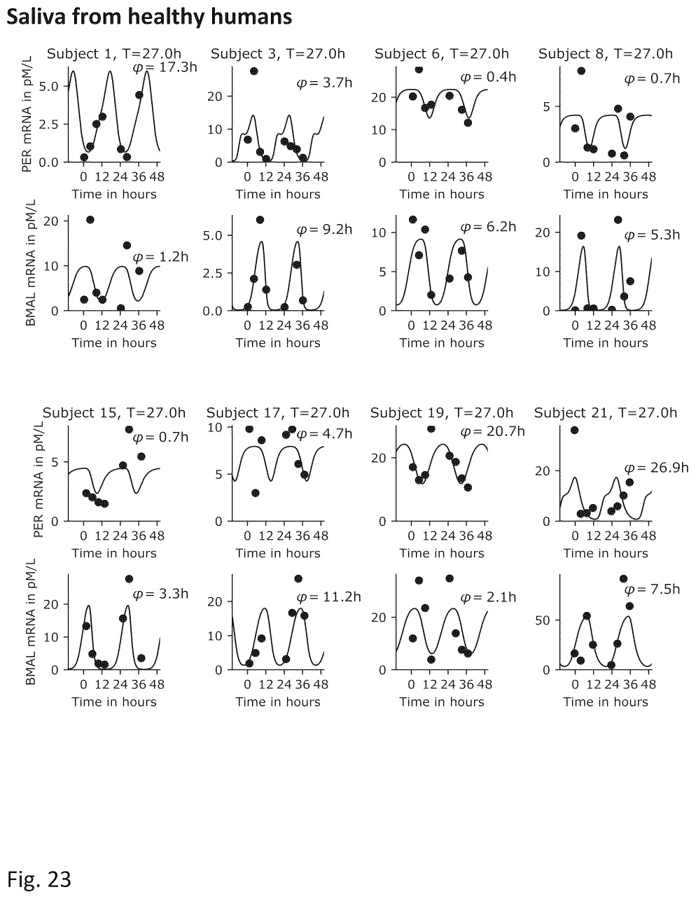

FIG. 23 illustrates circadian rhythms for a model fitted to saliva gene expression data of a set of healthy human subjects. The gene expression of PER2 (first row) and BMAL1 (second row) extracted from saliva (dots) is fitted by the mammalian transcription-translation network (lines). Phi states the phase of the modelled genes, i.e. the time of their maximum.

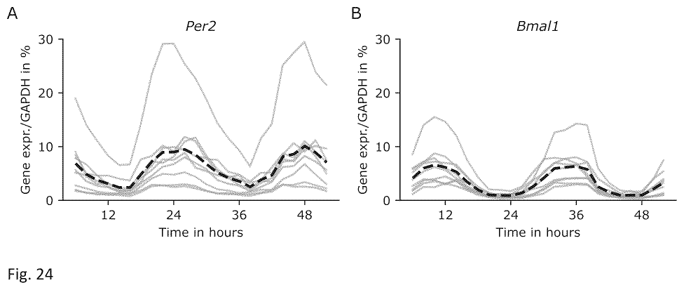

FIG. 24 illustrates the similarity of circadian oscillations in different mammalian tissues at the example of the circadian oscillation in Per2 and Brnall gene expression. Straight lines connect experimental measurements of aorta, adrenal gland, brown fat, heart, kidney, liver, lung skeletal muscle, and white fat over 48 hours, dashed curve is the resulting mean over tissues, representative of entrained mammalian tissue. Mouse were entrained by a 12:12 light:dark cycle and 12 h before timepoint 0 h released into constant darkness. Based on data first published by Zhang et al. 2014, accession numbers GSE54650 and GSE54652 [9].

FIG. 25 illustrates that saliva samples of representative healthy subjects (black dots) show similar trends as mammalian tissue (dashed). Timepoint 0 h corresponds to the mean wakeup time for subjects, and for the mammalian data to the start of the first activity period during constant dark.

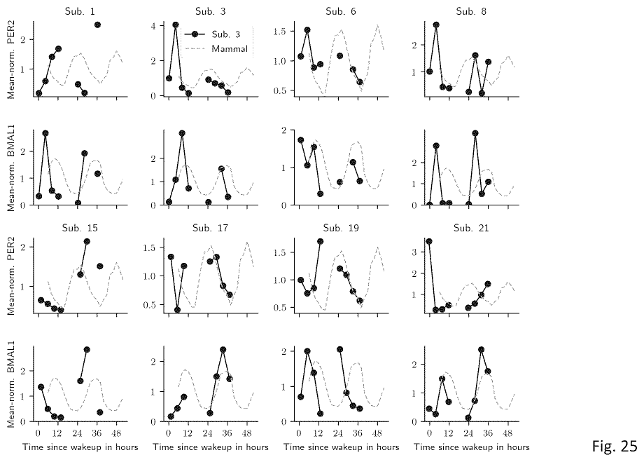

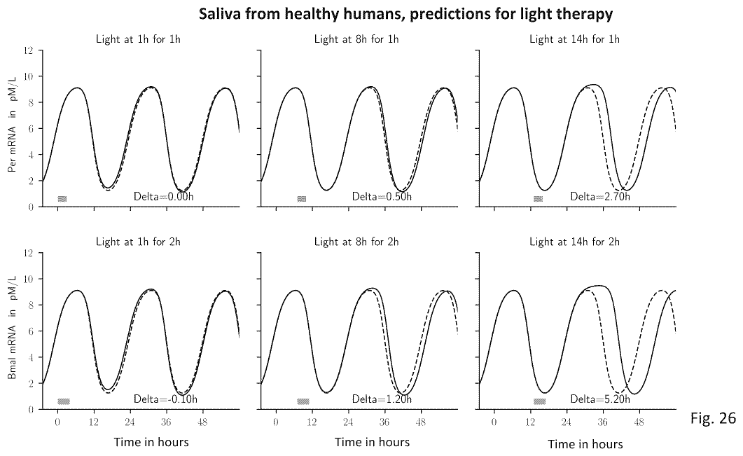

FIG. 26 illustrates that light therapy can induce changes in circadian gene expression in the mammalian core-clock model fitted to subject 6. Depending on light therapy starting time and duration, vastly different responses in the circadian rhythms (shown is BMAL1 expression) are observed. Grey bar marks light treatment, light therapy is implemented as a transient increase in PER2 transcription. Delta is the time difference between the phase expected without light treatment, and the phase observed with light treatment.

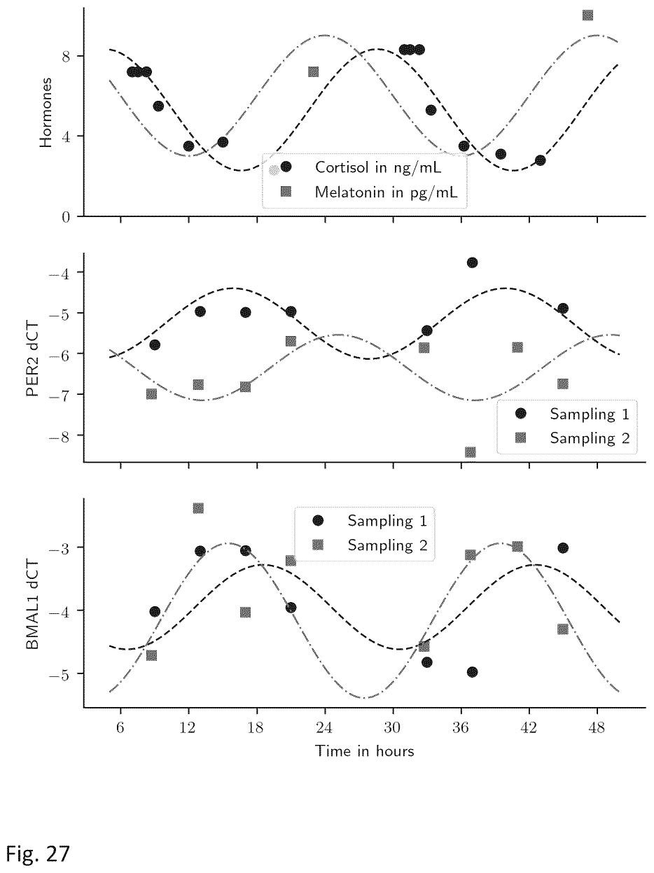

FIG. 27 illustrates cortisol levels and gene expression for one representative subject. Shown are experimental measurements as dots, and harmonic regression fits in interrupted lines with a period of 24 hours (p=3*10⁻⁶ for cortisol, not significant for gene expression). Sampling 1 and Sampling 2 were done with 3 months in between, the resulting gene expression profiles shows seasonal effects.

FIG. 28: Temporal mean of BMAL1 (A) and PER2 (B) expression versus melatonin values, considered for sampling day 1 and 2 separately. Coefficient of determination for BMAL1 is 0.05, coefficient of determination for PER2 is 0.87. Maximal gene expression of BMAL1 (C) and PER2 (D) versus melatonin values, considered for sampling day 1 and 2 separately. Coefficient of determination for BMAL1 is 0.04, coefficient of determination for PER2 is 0.69.

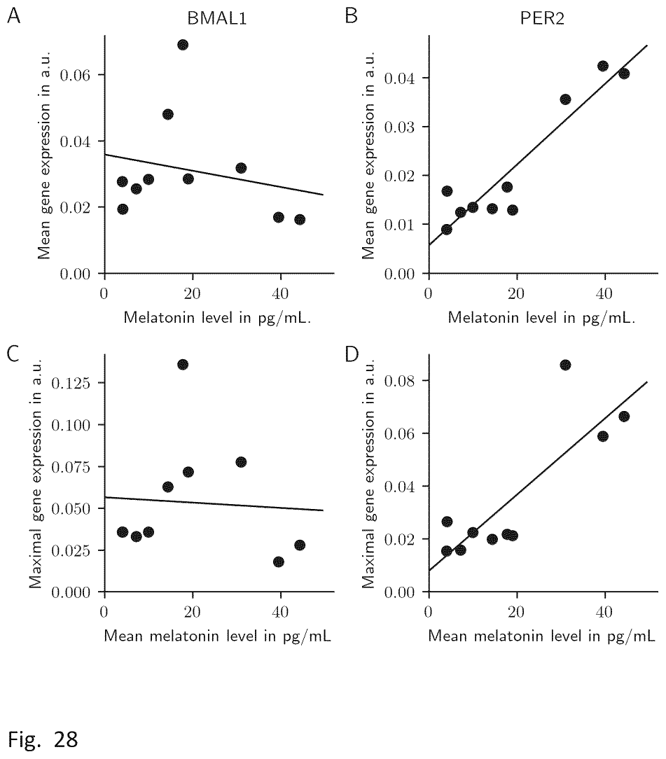

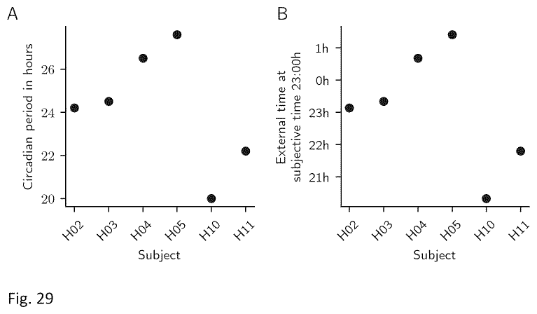

FIG. 29: Circadian time prediction. A Circadian period derived from the best fit of a·cosinor analysis to PER2 with periods between 20 h and 28 h. B Due to different circadian periods, subjects pass their subjective 23 h at different times of the day.

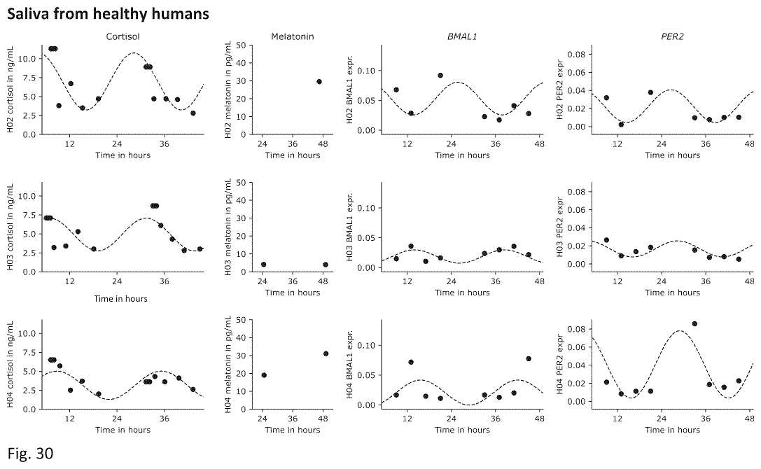

FIG. 30/31: Harmonic regression to cortisol values and gene expression using a period as predicted by the PER2 optimal period, the circadian period shown in FIG. 29

As indicated in FIG. 26 the present methods may also be used to show how the circadian profile of a patient or subject looks like and in one other embodiment (e.g. if problems in the circadian profile are detected), the methods and models of the present invention may be used to decide on the point of time to apply a measure or therapy to induce the clock, e.g. by light therapy or administration of melatonin. Such a measure or therapy may make the clock of the patient or subject more robust and could improve well-being.

As indicated in the Figures the RNA was extracted with RNA protect agent which is selected from the group comprising: EDTA disodium, dihydrate; sodium citrate trisodium salt, dihydrate; ammonium sulfate, powdered; sterile water, wherein a single reagent or a combination of different reagents may be used.

In one embodiment of the present invention it is preferred to provide 1.5 mL saliva and use of ratio of saliva to RNA protect agent of 1:1. Subsequently, RNA is extracted and RNA concentrations are measured.

The aim of the invention is to predict, optimal timing of behavior, more specifically the timing of best sports performance, possibly to monitor (over time) the circadian rhythms and adjust the timing it if needed. Previous studies focused on predicting the circadian time which means a 24 hours-rhythm. However, given that the prediction of the circadian time is in an application used for a second prediction, the prediction of the timing of behavior, the error accumulates with each prediction. The present invention instead relies on a direct measurement of the behavioral relevant timing directly based on the genetic expression. That means if the genes have a 20 h, or 30 h or 12 h rhythm in expression, the method of the present invention would also be able to detect that. These would be non-circadian rhythms and include infradian and ultradian rhythms. Thus, the present invention assesses and monitors the circadian profile. The circadian profile could be a circadian or non-circadian rhythm.

Thus, in addition to the core-clock genes levels of cortisol and/or melatonin may be used and fitted into the methods and models according to the invention. Cortisol or melatonin hormone levels were measured using commercial kits from cerascreen (Cortisol Test and Melatonin Test Kits) and by providing saliva samples at different times of the day (Cortisol Test Kit) or before sleep (Melatonin Test Kit) according to the manufacturer's instructions. Samples were sent to cerascreen laboratory for the detection of hormone levels (via immunoassay, e.g. radioimmunoassay or ELISA) and results were provided after the analysis.

The expression profile allows to relate gene expression to melatonin levels. The coefficients of determination from FIG. 28 suggest no correlation for melatonin with BMAL1, but a correlation between PER2 mean expression and Melatonin level, and potentially a weaker correlation between PER2 maximal expression and melatonin level. This relates a saliva derived gene-based measure with a hormonal level set by the central clock in the SCN.

The circadian profile extracted from the saliva samples is also fitted to predict circadian time, see FIG. 29.

Thus, in addition to the core-clock genes levels of cortisol and/or melatonin may be used and fitted into the methods and models according to the invention. Cortisol or melatonin hormone levels were measured using commercial kits from cerascreen (Cortisol Test and Melatonin Test Kits) and by providing saliva samples at different times of the day (Cortisol Test Kit) or before sleep (Melatonin Test Kit) according to the manufacturer's instructions. Samples were sent to cerascreen laboratory for the detection of hormone levels (via immunoassay, e.g. radioimmunoassay or ELISA) and results were provided after the analysis.

The expression profile allows to relate gene expression to melatonin levels. The coefficients of determination from FIG. 28 suggest no correlation for melatonin with BMAL1, but a correlation between PER2 mean expression and Melatonin level, and potentially a weaker correlation between PER2 maximal expression and melatonin level. This relates a saliva derived gene-based measure with a hormonal level set by the central clock in the SCN.

The circadian profile extracted from the saliva samples is also applicable to predict circadian time, see FIG. 29.

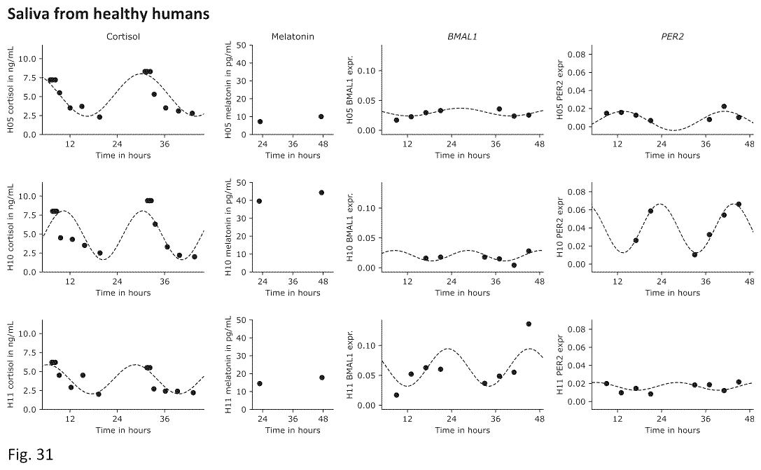

Because of the correlation between melatonin and PER2, according to the invention PER2 may be used to derive the circadian period of the subject, see FIG. 29A, from the optimal period to fit PER2 gene expression with a harmonic regression. The circadian profile extracted from the saliva samples is also fit to predict circadian time based on the derived period, see FIG. 29B. Using the individual circadian periods, the hormonal and gene expression profiles may be fitted by harmonic regressions, see FIGS. 30 and 31. This may be used as a test whether the extracted period of the subject indeed fits all its circadian profiles.

According to the present invention “assessing the circadian or non-circadian rhythm” or “assessing the athletic performance” also includes “monitoring the circadian or non-circadian rhythm” or “monitoring the athletic performance”. “Monitoring” means at least twice “assessing”.

As an objective measure, gene expression may be quantified four times a day (the times mentioned in this disclosure serve as an e.g. of possible sampling times), two days in a row. In particular, four samples of saliva may be taken on two consecutive days, and the gene expression of selected genes in accordance with the present invention is determined in each of the samples. While other studies have focused on a prediction of circadian time (exact estimation of precise internal time), with the aim to allow for a subsequent prediction of the optimal time for a behavior, such as high sports performance, the present invention focuses on a direct prediction of the relevant timing including a circadian profile, without the deviation through circadian time. This means previous studies attempted to tell the exact internal time. The present invention provides a full 24 h profile, it may provide a 48 h profile, if measured during two consecutive days, each day e.g. 4 saliva samples are taken. If more samples are taken over more days longer profiles may be provided.

The computational analysis of the measured gene expressed obtained from the saliva samples as set forth above can be separated into three different approaches, which will be discussed in the next sections. First, experimental data can be fitted with a periodic function, in order to establish oscillatory behavior and extract oscillation properties. Machine learning can then be used to predict the timing of behavior based on the gene expression. Modeling the molecular network underlying the circadian rhythm as well as the behavior under consideration can add information. Background and general considerations in view of the present invention and the overall process will be explained first. Following, the procedure according to the present invention will be described with respect to the specific application.

A general problem in chronobiology is the screening for circadian oscillations in data, such as in the series of eight data points obtained from the saliva samples. It has to be determined whether the observed variation is due to some circadian rhythm, or only due to noise. To distinguish oscillating from non-oscillating measures, a periodic, non-constant function is fit to the data, and if the fit is significant, the measure is considered oscillatory. Successful fits allow to read off the oscillation phase, amplitude and period. Fitting the oscillatory data by curve fitting is described below.

If a trigonometric function is fit to the data, this is called harmonic regression, which may be done as set forth in the following. It will be appreciated that this is described by way of example only. Below, other approaches are briefly outlined. Circadian rhythmicity of genes may be tested (significance e.g. bounded by a fit with p-value<0.05) and circadian parameters (phase and relative amplitude) may be determined for sample sets with at least 7 data points (3 hours sampling interval) for a period range of 20 to 28 hours with a 0.1 hour sampling interval by fitting a linear sine-cosine function to the time-course data (ΔΔCT normalized to the mean of all time points), for instance using known tools, e.g. the R package HarmonicRegression (Luck et al. 2014). The harmonic regression procedure fits the model y(t)=m+a·cos(ωt)+b·sin(ωt) in order to estimate absolute amplitudes (A=√(a2+b2)) and phases (φ=a·tan2(b,a)) along with confidence intervals and p-values (Luck et al. 2014). The fit uses a least-squares minimization. Extensions to this fit method are reviewed in as cosinor-based rhythmometry in (Cornelissen 2014).

A combination of sine waves are also used by other rhythmicity detection methods (Halberg et al., 1967; Straume, 2004; Wichert et al., 2004; Wijnen et al., 2005; Thaben and Westermark, 2014). Yet, Fourier-based methods can have the drawback that they require evenly sampled data. Other alternatives are named in the following. It will be appreciated that the invention is not limited to these packages but any other suitable method for fitting a periodic function to the measured gene expression data may be applied.

The software-packages RAIN (a robust nonparametric method for the detection of rhythms of prespecified periods in biological data that can detect arbitrary wave forms (Thaben and Westermark 2014), which improves on older methods: a nonparametric method implemented as the program “JTK CYCLE”, which assumes symmetric curves (Hughes et al., 2010), as well as its improvement eJTK CYCLE that includes multiple hypothesis testing and more general waveforms (Hutchison et al. 2015)[Ref: Hutchison A L, Maienschein-Cline M, Chiang A H, et al. Improved statistical methods enable greater sensitivity in rhythm detection for genome-wide data. PLoS Comput Biol 2015;11:e1004094.], while the HAYSTACK method (Michael et al., 2008) can also detect chain saw type rhythmicity, but relies on a small set of predefined wave form alternatives and is thus not really general.

BIO_CYCLE: “We first curate several large synthetic and biological time series datasets containing labels for both periodic and aperiodic signals. We then use deep learning methods to develop and train BIO_CYCLE, a system to robustly estimate which signals are periodic in high-throughput circadian experiments, producing estimates of amplitudes, periods, phases, as well as several statistical significance measures.” (Agostinelli et al. 2016).

A modified version of the empirical Bayes periodicity test to detect periodic expression patterns (Kocak and Mozhui 2020). Their results demonstrate that this approach can capture cyclic patterns from relatively noisy expression data sets.

Especially to find higher harmonics, some studies have exploited Fisher's G-test and COSOPT jointly to recognize rhythmic transcripts, classified, depending on the length of the oscillation period, as circadian (24±4 h) and ultradian (12±2 h and 8±1 h) (Hughes et al. 2009; Genov et al. 2019).

Once the curve fitting to the measured data points has been done, machine learning methods are applied to predict circadian time for human subjects, which has in principle been proposed by several studies. Some example studies are outlined to provide background for the process of the present invention, which will be described in detail thereafter.

While the present invention focusses on studies based on gene expression, continues measures of light exposure and skin temperature as well as metabolites from blood or breath sampling can be used to predict circadian time (Kolodyazhniy et al. 2012; Kasukawa et al. 2012; Sinues et al. 2014).

Also skin temperature in combination with questionnaires and activity measurements can predict circadian time, by a method called INTime (Komarzynski et al. 2019). The following studies predict circadian time or time-of-the-day from gene expression data extracted from human blood:

BioClock (though only mouse data so far) (Agostinelli et al. 2016): Normalization is Z-score data (subtraction of mean and then divided by standard deviation—this removes any amplitude information), their method is a deep neural network, they use BioCycle to derive rhythmicity, and standard gradient descent with momentum to train the network, the original publication uses different tissues but only from mice.

ZeitZeiger (Hughey 2017): Data normalized and batch-normalized. Discretized and scaled spline fits are used to calculate sparse principal components (SPC), predictions based on fitted splines to SPC with maximum likelihood. They use 15 genes from human blood, only two of those are part of the core-clock. Due to the batch-normalization, the incorporation of new data requires retraining of the algorithm, their algorithm was improved for humans. One sample is enough for predictions.

Partial least squares regression (PLSR) (Laing et al. 2017): Training data is batch-corrected and quantile normalization is applied. No batch correction on test set, to prevent the need for retraining whenever new data is added. Their algorithm uses 100 genes out of 26,000 available ones from blood. One sample is enough for predictions, more is better.

TimeSignature (Braun et al. 2018): Mean-normalized genes, algorithm is optimized with a least squares approach plus elastic net for regularization. They use 40 genes from two samples of blood, 12 h or less apart. This study seems to generalize well, it was validated in 3 studies, one of them with a different experimental method to measure gene expression.

BodyTime (Wittenbrink et al. 2018): ZeitZeiger (see above)+NanoString platform (an experimental machine which allows for high quality counts of gene abundance without the need of an amplification step, thus it measures the original abundances). They use 12 genes from human blood, but also get good predictions for as few as 2 genes (one of which is PER2 which also is used in the sports study). Their algorithm is validated in 1 independent study that uses the same method.

TimeTeller, preprint (Vlachou et al. 2020): They aim to predict clock functionality from a single gene sample. Application to breast cancer, showing that their prediction relates to patient survival. Rhythmicity and synchronicity were analysed to choose a set of 10-16 genes used for the prediction (all core-clock or clock-controlled). Their algorithm is trained with a set of repeated samples and extracts from them the probability to observe a particular gene expression profile given some time t. The prediction inverts this information; they use a maximal likelihood function to predict for a given gene expression profile the time t. A model of the core-clock was used to test their algorithm.

Machine learning can be used to predict some output based on a (high-dimensional) input consisting of a set of so-called features, i.e. the different dimensions of the input space. A set of input-output pairs is used to train the algorithm, i.e. the algorithm performs some kind of optimization that allows it to optimally predict the output based on the input. This set is called training set. To evaluate the performance of the algorithm, it is fed with an independent set of inputs from a so-called test set, while not presenting the associated outputs. The predictions of the algorithm are then compared to the associated outputs, and the number of correct predictions is counted. Several measures can be used to quantify prediction quality; for instance the accuracy, i.e. the number of correct predictions divided by the total number of predictions, may be used. For small data sets, as typical with human subjects, the separation of the data into two independent training and test sets means that it would not be taken advantage of the full amount of information available for the prediction. The solution is cross-validation, for which the total set is repeatedly separated into different training and test sets. Especially for very small data sets, one can use all but one subjects to form the training set, and test the algorithm only on the left-out subject. This is called leave-one-out cross-validation (sometimes also leave-one-subject-out cross-validation). Given n subjects, the training on all-but-one subjects is repeated n times, such that each subject has been once selected as test set. The accuracy of the prediction is in this case calculated as the number of correct predictions over n.

While the application of machine learning to genetic data is generally known, the benefits and value of the results highly depend on the input data. Thus, the inventors put their focus on the input of the data, both via normalization and presentation of derived features, and also on understanding in detail what information the algorithm uses. Most published studies focus on their machine learning algorithm, why it is best suited for the prediction at hand, while mentioning their data preparation only on the side, and their discussion of the workings of the algorithm is often restricted to a single evaluation approach (for example, showing that a restricted set of genes is most relevant for prediction, but without stating which characteristics of the genes are important).

When comparing the performance of different machine learning algorithm on standard training and test sets, their performance differs often just in a few percent—an order of magnitude hardly relevant for biological data, which often consists of only few samples with a high level of noise compared to other typical machine learning applications. It has been found that the particular algorithm will not make much difference, but what makes a large difference is the way in which the input is prepared for machine learning. Many machine-learning algorithms are optimized for data with zero mean and a standard deviation of one for each dimension individually. Dimension-wise normalization makes however no sense for time-series data, where dimensions are not independent of each other, but where the information on the temporal development can only be accessed by comparing different dimensions.

As mentioned above, eight saliva samples may be collected, preferably distributed over the day, e.g. at 9 h, 13 h, 17 h and 21 h over two consecutive days. This results in a feature space with 8 dimensions. Instead of normalizing each of these dimensions independently over all subjects, the data may be normalized by their temporal mean for each subject independently. In addition, this subject-wise mean normalization of each gene has the advantage of keeping the temporal structure of the data intact (phase and relative amplitude of the oscillation), thus preserving this information for the machine learning algorithm. Yet, what is lost by this normalization is the mean values of the oscillations, and thus also their relative expression mean. To preserve this information for the machine-learning algorithm, this may be added as additional features to the feature space. Subject-wise normalization has to be reconsidered when the molecular profile of a subject was measured repeatedly over a longer interval of time. For the example of multiple measurements during disease progression, we have already published one possibility to normalize data from subsequent molecular profiles such that the result can be compared between sampling dates and even between different experimental procedures (Yalcin et al. 2020).

In many cases, machine learning algorithms are considered as “black boxes”. However, as the machine learning algorithms are faced with noisy biological data, derived of a system where lots of additional information are available, the approach of the present disclosure is to let the machine learning optimize the prediction, but then to uncover the underlying information flow from input to prediction output, with the aim to double-check the generalizability of the solution found by the machine learning. Optimally, any additional information can be added as either input to the machine learning or as constraints (in form of a cost function), but in a first step, the formulation of these inputs and constraints is more difficult than to take the algorithm and check a posteriori whether any additionally known information is violated.

Once the prediction was done using the complex feature space created in the step explained above, a simplification of the feature space may be carried out. This serves to identify the relevant features. For example when predicting the optimal sports time it may be tested whether the peak time of the genes would suffice for the prediction. It was found that this was not the case, i.e. the algorithm uses more than this information. In general, dimensionality reduction methods may be used first, which results in fewer, new features that are combinations of the original features. Then it may be tested which combinations of individual original features is sufficient for successful predictions, and compare whether that fits the features which are dominant in the features resulting from dimensionality reduction. This is an important step to understand based on which information the prediction is made by the algorithm, which is relevant to double check its generalizability to new data.

Related to the first point, machine-learning algorithms are preferred which may be called interpretable, i.e. they provide some information on the prediction. Examples for such algorithms are sparse principle components analysis as used as an intermediate step in (Hughey 2017), and partial least squares regression, as used in (Laing et al. 2017). In both cases, the prediction is made based on a combination of the features into few most informative features, and it is for example possible to plot two of them against each other in order to see how the data of the training set and test set is distributed in these features. It is expected that subjects with optimal times that are neighboring are also neighbors in this component space. If this is not the case, the algorithm is unlikely to generalize well.

Then, prediction performance may be benchmarked using a neural network model, which may be used as an approximation for an upper border of prediction performance. Neural networks do not require normalization, as they are universal computing machines and can hence implement the optimal normalization for the problem at hand on their own. However, this is at the same time the problem with neural networks. As they decide for themselves which are the relevant features of the data, there is no controlling whether they use biologically relevant information, or noise information that—by chance—fits the prediction. Furthermore, their high flexibility facilitates overfitting of the data and the resulting algorithm are difficult to interpret, such that we cannot a posteriori enhance our trust in the method by understanding the information flows from input to prediction output. Despite these disadvantages, neural networks may be used at least as benchmark algorithms, to test which performance can be expected when the information is provided without constraints. The present invention aims at providing an algorithm with a performance similar to that of the neural network, but not by means of overfitting the experimental data, as suspected for the neural network, but by means of focusing on the biologically relevant information.

A linear support-vector-machine (SVM) can be used to predict two different outputs based on a high-dimensional input data (see below for details). Linear SVMs are extremely simple compared to the non-linear methods explained above. They have the advantage of a fast implementation, and, as their complexity is low, they are not so prone to overfitting. For these reasons, a linear SVM may be used to predict the optimal sports timing, and it turned out that this was sufficient for prediction. However, it is noted that the prediction problem was “linearly separable”, and as there is no reason to assume that any application is “linearly separable”, it may be preferable to use in general non-linear methods. Yet, testing how well a linear model performs compared to the non-linear model can help to benchmark how much complexity is needed for the prediction. For example, if a linear model results in an accuracy of 0.85 and a non-linear model in an accuracy of 0.9, it is probably not worth using the non-linear model for the application, as it performs only slightly better on the test set, but has a larger probability of overfitting the data, which might lead to less performance on a new set of data. If the difference is larger, a non-linear model is likely more appropriate.

As mentioned above, linear support-vector-machine (SVM) can be used to predict two different outputs based on a high-dimensional input data. For training, the linear SVM is fed with multi-dimensional input data and a binary output. The training set consists of n subjects, and the input with p dimensions is denoted as xᵢ∈R^{p}, $i. The output yᵢ is encoded as −1 for the first type of output and as +1 for the second type of output, y∈1, −1ⁿ. The training of the SVM fits a hyper-plane into the input space such that it separates the two output types as best as possible and such that the distance to the input data points is maximal.

Mathematically, the following minimization problem is solved:

$${min}_{w , b} \frac{1}{2} {w}^{T} w + C {\sum }_{i = 1} max ( 0 , {y}_{i} ( {w}^{T} ϕ ( {x}_{i} ) + b ) ) ,$$

where ϕ is the identity function, (w^{T}ϕ(xᵢ)+b) is the predicted output for the ist input. For the application to the sports data, the regularization constant C is set to 1.0 (default of the python implementation).

Predictions for some input xₜₑₛₜ then be calculated as w^{T}ϕ(xₜₑₛₜ) b with the w and b resulting from the above minimization, and compared with the correct output. Leave-one-subject-out cross-validation implies that this step is repeated n times, each time with another participant removed to form the training set.

With regards to the data obtained from the saliva samples, in accordance with an example of the present invention, the machine learning requires optimally eight timepoints, four is less optimal: Using the two-day measurement of PER2, consisting of eight data points, a linear support vector machine can predict early versus late HST sports performance with an accuracy of 1.0 (100% correct predictions). The accuracy drops to 0.8 or 0.4 (80% or 40% correct predictions), if the prediction is based on only the first or the second day with four data points each (as the machine learning cannot handle missing data points, those are thereby filled with appropriate values: the expression of PER2 is set to zero if ARNTL (BMAL1) was measured successfully while there was too little PER2 to be detectable in the experiment, and the value of the other day was used if the whole measurement was unsuccessful).

The present invention provides a methodology for the detection of circadian rhythms based on saliva sampling, which is introduced as a non-invasive and practical approach. While this methodology may be particularly beneficial for future sports studies, it may be useful for more general applications and for anyone, for example anyone who just wants to know or to follow up their circadian profile e.g. across the years or across the seasons. The methodology relies on the fact that ARNTL (BMAL1) and PER2 expression shows daily changes in human blood, hair and saliva cells, which are distinctive for every individual tested. Also sport performance displays daily variations, e.g. between 09 h, 12 h, 15 h, and 18 h, and peak performance is time-of-day dependent, with different optimal timing for strength exercises compared to endurance exercises. Biomechanical muscle properties in resting muscles undergo daily fluctuations, which correlate with sport performance and clock gene expression variations in saliva. Therefore, the method of the present invention utilizes salivary gene expression of ARNTL (BMAL1) and PER2 as personalized predictors of athletic fluctuations and individual peak times in performance.

The sample collection can be performed in almost any location. The samples of saliva are collected at the predetermined points in time in a tube containing an RNA stabilizing reagent followed by RNA extraction as described below. In order to minimize RNA degradation through material transfer, according to a preferred exemplary method an amount of 1 mL of unstimulated saliva may be collected directly into a 5 mL Eppendorf tube containing 1 mL of a non-toxic RNA-stabilizing reagent called RNAprotect Tissue Reagent (Qiagen) which should be mixed immediately to stabilise the saliva RNA. The direct addition of saliva to the RNA stabilizing reagent, which is mixed immediately, was found to generate good quality/quantity RNA suitable for gene expression analysis and by using 5 mL tubes instead of 2 mL tubes (wider opening for sample collection), the sampling procedure was more comfortable to perform. Other tested sampling protocols had shown to lead to poor quality and quantity of RNA that was not suitable for the downstream application. For example, 200 μL saliva was collected in a 50 mL tube on ice, which was immediately transferred to a 2 mL tube containing 1 mL RNA stabilizing reagent followed by RNA. In another protocol, 10000 μL saliva was collected in a 50 mL tube and processed as described above, in which the extracted RNA did not pass the desired quality/quantity either.

With only four sampling time-points per day (9 am, 1 pm, 5 pm and 9 pm) and over two consecutive days, it is possible to assess precise circadian rhythms in gene expression of any individual. For best sampling quality, the individuals should refrain from eating and drinking one hour prior to sample collection. Individuals can optionally wash their mouths with water five minutes before sampling without swallowing the water. The stabilized samples can be kept at room temperature for a few days, optimally at 4° C. during several weeks, for posterior molecular analyses via different possible methods, such as RT-qPCR, Nanostring, microarrays, and sequencing. In one embodiment any other method known by a person skilled in the art to measure gene expression could be used. One could of course do the same with protein expression instead of gene expression in principle.

A method for RNA extraction from saliva samples is provided to effectively extract RNA, preferably using TRIzol (Invitrogen, Thermo Fisher Scientific) and the RNeasy Micro Kit (Qiagen). It has proven to be particularly beneficial to use a combination of both, rather than only one of them (typically either TRIzol or RNeasy Kit is used). For this, the samples were centrifuged at 10,000×g for 10 min at room temperature to generate cell pellets. The supernatant was removed and the pellets were homogenized with 500 μL TRIzol followed by the addition of 100 μL chloroform and mixed for 15 sec at room temperature. After a 2 min incubation at room temperature, the samples were centrifuged at 12,000×g for 15 min at 4° C. The mixture will separate into a lower red phenol-chloroform phase, an interphase, and a colourless upper aqueous phase. The upper aqueous phase contains the RNA, which was transferred into a new 2.0 mL microfuge tube using a 1 ml pipette with filtered tip, being careful not to transfer any of the interphase layer. After the transfer, the samples were processed according to the manufacturer's instructions of the RNeasy Micro Kit (Qiagen) on a QIAcube Connect device (Qiagen). Finally, the RNA is eluted in RNA-free water and can be used directly for gene expression analysis. It has been developed the secondary purification and elution step of the saliva RNA using RNeasy Micro Kit in order to:
a) Increase sample quality and purity, which is necessary for downstream applications and is otherwise lost with the traditional and commercial methods, since the RNA content in saliva is low.
b) Automate the sample processing and RNA extraction using the QIAcube Connect automation device. With this, it is possible to perform sample handling much faster and reduce sample contamination induced by human errors.

In one embodiment, gene expression analysis is carried out via cDNA synthesis and RT-PCR as follows. For RT-qPCR analysis, the extracted RNA is reverse transcribed into cDNA using M-MLV reverse transcriptase (Invitrogen, Thermo Fisher Scientific), random hexamers (Thermo Fisher Scientific) and dNTPs Mix (Thermo Fisher Scientific). RT-PCR is performed using SsoAdvanced Universal SYBR Green Supermix (Bio-Rad laboratories) in 96-well plates (Bio-Rad laboratories). The RT-PCR reaction is performed using a CFX Connect Real-Time PCR Detection System (Bio-Rad laboratories) using primers from QuantiTect Primer Assay (Qiagen) as well as custom made primers.

The experimental data obtained from the saliva samples as explained above will be further analysed with a computational model in order to provide scientifically justified and personalized suggestions for best timing of sports (wherein applications for other certain daily activities, such as light exposure, sleep, food and medicine intake may be envisioned), to avoid circadian rhythm disruption, and thus enhancing health. As will be described in detail below, a mathematical model for the circadian clock is created, which may include core-clock and clock-controlled metabolic genes in about 50 elements, based on which models for relevenat gene networks, particularly related to physical performance in connection to the circadian clock can be generated. By feeding each network with specific gene expression data obtained from saliva samples, accurate predictions for day-/night-time activities can be generated.

Based on the experimental data from the saliva samples, more specifically the measured gene expressions and the resulting fitted oscillatory curves, a core-clock model will be generated, which may include a larger number of other genes that were not included in the measurements but that may be relevant for the desired prediction (this model is also referred to as “network computational model” in this disclosure). The core-clock is located in the brain (suprachiasmatic nucleus) and its oscillations entrain the peripheral clocks. The oscillations result from feedback loops, which can be investigated by experimental and theoretical means.

Rather than relying only on a model for the circadian rhythm that simply shows oscillations such as phase-oscillators, the present disclosure uses a molecular model, which models (part of) the molecular interactions underlying the circadian clock. This is because, as already mentioned, molecular models contain biological information that might be useful for predictions. Molecular models with simple feedback loops models are often based on Goodwin's oscillator, e.g. (Ruoff and Rensing 1996), but the level of detail may also be extensive (Forger and Peskin 2003). According to an exemplary embodiment of the present invention, a model at an intermediate state of complexity is generated, complex enough to capture a significant part of the genetic network, but not too complex, as this may affect fitting of the data to the model without significant overfitting. Relogio et al. have published a model at this level of complexity, with 19 dynamical variables, which is used in the following (Relogio et al. 2011).

Now referring to FIG. 2, two examples of fits of the saliva data to the core-clock model are shown. (Relógio et al. 2011). Left: Saliva data is plotted as dots, including data for ARNTL (BMAL1) and PER2, where the measurements of both measured days within the same 24 hours are plotted, which is then plotted for two consecutive days. The curves result from the model fit. Middle and right: The model contains 19 dynamical variables, 17 are plotted in these two panels.

FIG. 2 illustrates that the dynamical model may restrict the shape of the fitted curves. In this exemplary embodiment, the curves are more complex than a simple sine-cosine function, but they are also not perfectly fitted to the data, as may happen when a spline is used to fit the data, because the model can only produce shapes that result from the interacting dynamics.

The data base for the fit are the experimental, non-logarithmic gene expression values, 2^{ΔCT} In order to get the experimental values on the same scale as the model output (which is on the order of one), the gene expression of both PER2 and ARNTL (BMAL1) are normalized in this exemplary embodiment by the mean of ARNTL (BMAL1) expression; that way the relative amplitude of both genes is preserved. In order to allow for a fairer comparison both the simulated and the experimental data may be normalized by their respective mean ARNTL (BMAL1) expression.

The complexity of the model with around 80 parameters makes a meaningful fit that prevents overfitting challenging. At least one of or a combination of one or more (including all) of the following approaches may be used to fit the model to the saliva data. It will be appreciated that other approaches may be used alternatively or in addition to adjust the model. To constrain the model to parameter regions in which continuous oscillations occur, a bifurcation analysis may be used to delineate the regions with limit cycles (i.e. continuous oscillations), and restrict the parameter optimization to these regions. This prevents fits that show a (slow) relaxation to a steady state, as the model may be expected to have a stable limit cycle. Considering the bifurcation structure, fits will be faster because less parameters need to be checked and because less simulation time is required to ensure relaxation of the oscillatory behavior.

To minimize the number of parameters with large deviations from the original model, standard regression methods may be used that are also added to the cost function, such as ridge regression, which has proven useful so far.

Based on biological and dynamical considerations, certain parameters may be fixed at the original value and exclude them from the fit. This may be for example done for parameters that show only minor impact on the resulting curves, parameters for which no inter-individual variation is expected (i.e. diffusion constants which result from biochemical properties) and parameters which have been repeatedly measured in experiments for humans.

Finally, least-squares minimization (details see below) may be used to minimize the distance between experimental data and fitted curve. The associated cost function used for least-squared error minimization may be extended with additional terms that can restrict the period (should be between 20 and 28 hours for human material), amplitude (no constant amplitude, e.g.) and the position of peaks and troughs.

According to an exemplary fitting procedure, the two days of gene expression data are interpreted as replicates, and the model is fitted to both data points for 9 h, 13 h, 17 h and 21 h at the same time, as indicated by two data points for each time point in the above figure. In order to fit the model to the gene expression data, the model may be run for 72 hours with a time resolution of 0.01 hours. The last 48 hours are used for the analysis. As model and experiments have no common time, all possible time-shifts between experiments and model output are considered (0 up to 24 hours). For each shifted variant of the model output, the least-squares cost function C may be calculated between the experimental values xₑₓₚ and the model output x_{mod} as C₀=2\sqrt{1+(xₑₓₚ−x_{mod})²−2} for both genes (ARNTL (BMAL1) and PER2), and then the time-shift with the minimal summed cost for both genes may be selected. To optimize the fit, a selection of the following additional cost function terms may be added to C₀:
A regression term, that penalizes large parameters: Given the parameter vector p=(pᵢ) where i is between 1 and the number of parameters, ridge regression adds a term C_{ridge}=cΣpᵢ² to the cost function, with a prefactor c that was set to 1.
A term that penalizes deviating periods. One may first measure the period p of the model, and then compare it to the standard human circadian period of 24.5 hours: Cₚ=cₚ(p−24.5)², with weighting factor c p=1.
A term that penalizes the amplitude deviations: For experimental and simulated amplitudes Aₑₓₚ and Aₛᵢₘ, the cost function is Cₐ=cₐ(Aₛᵢₘ−Aₑₓₚ)², with weighting factor cₐ=0.05.
A term that penalizes if the peak or trough position deviates. Peak times of the experimental data tₑₓₚ and of the model trace with the optimal time-shift applied trim may be derived and the cost term calculated as Cₜₒₚ=cₜ(p−24.5)², with weighting factor cₜ=0.1 for the peaks. The same may be done for the trough, resulting in a cost C_{down}, with weighting factor c_{d}=0.05.

The cost function sums the individual costs weighted by a factor chosen to optimize the influence of each cost. Cₜₒₜₐₗ=C₀+C_{ridge}+Cₚ+Cₜₒₚ+C_{down}.

In one embodiment the present invention provides a method of assessing the circadian rhythm or circadian profile of a subject and/or assessing and predicting the athletic performance of said subject, wherein said method comprises the steps of: Providing at least three samples of saliva, more preferably four samples of saliva, from said subject, wherein said samples have been taken at different time points over the day,
Determining gene expression of at least two members of genes for the core-clock network, in particular of at least two members of the group comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, in each of said samples, and
Assessing and predicting by means of a computational step based on said expression levels of at least two members of the groups comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, over the day the circadian rhythm of said subject and/or the individual diurnal athletic performance times.

In a second embodiment the present invention provides a method wherein the gene expression is determined using a method selected from quantitative PCR (RT-qPCR), NanoString, sequencing and microarray.

In another embodiment the present invention provides a method wherein the gene expression is determined using quantitative PCR (RT-qPCR).

In another embodiment the present invention provides a method wherein the gene expression is determined using NanoString.

In another embodiment the present invention provides a method which allows assessing the circadian rhythm of said subject comprises determining a periodic function for each of at least two members of the groups comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, that approximates said expression levels for each of at least two members of the groups comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, preferably comprising curve fitting of a non-linear periodic model function to the respective expression levels, wherein the curve fitting is preferably carried out by means of harmonic regression.

In a further embodiment the present invention provides a method wherein the computational step comprises
processing the determined expression levels and/or the respectively fitted periodic functions to derive characteristic data for each of at least two members of the groups comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, said processing comprising determining the mean expression level of expression of at least two members of the groups comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, and normalizing the expression levels using the mean expression level.

In a further embodiment the present invention provides a method wherein said characteristic data comprise:
the amplitude of change of expression of a gene, and/or the amplitude relative to one of the other genes, and/or
the mean expression level of expression of a gene, and/or the mean relative to one of the other genes, and/or
the peak expression level of a gene, and/or the peak relative to one of the other genes, and/or
the amplitude of change of expression of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC over the day, and/or
the relative difference of the amplitudes of change of expression of any two of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC, and/or
the mean expression level of expression of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC, and/or
the relative difference of the mean expression levels of expression of any two of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC, and/or
the peak expression level of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC over the day, and/or
the relative difference of the peak expression levels of any two of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC, and/or
the time of the peak expression level of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC,
the relative difference of the times of the peak expression level of any two of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC,

wherein the amplitude, period and phase expression level of expression of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC are extracted from the determined expression levels and/or the respectively fitted periodic function.

In a further embodiment the present invention provides a method wherein from the characteristic data only the timing of the peak expression level of PER2 and the mean expression level of BMAL1 are used in said computational step.

In a further embodiment the present invention provides a method wherein the computational step further comprises
fitting a network computational model to the derived characteristic data that comprises a representation of the periodic time course of the expression levels for each of at least two members of the group comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, as well as a representation of the periodic time course of the expression level for at least one, preferably a plurality of further gene(s) included in a gene regulatory network that includes said at least two members the group comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2; and/or
training a machine learning algorithm on the derived characteristic data of the network computational model, particularly optimize in terms of the representation of the periodic time course of the expression level for the at least one further gene.

In another embodiment the present invention provides a method which allows assessing and/or predicting the individual diurnal athletic performance times comprises in the computational step fitting a prediction computational model on data obtained from said fitted periodic functions and/or said network computational model, wherein the prediction computational model is based on machine learning, including at least one classification method and/or at least one clustering method wherein said method(s) are preferably selected from the group comprising: K-nearest neighbor algorithm, unsupervised clustering, deep neural networks, random forest algorithm, and support vector machines.

In a further embodiment the present invention provides a method wherein additional physiological data of the subject are provided for fitting the prediction computational model.

In a further embodiment the present invention provides a method wherein the oscillation amplitude and/or peak time of the individual diurnal athletic performance during the day are assessed and/or predicted, wherein predicting the peak time of the individual diurnal athletic performance preferably comprises selecting at least one period of time from at least two distinct periods of time during the day as the peak time.

In another embodiment the present invention provides a method wherein the network computational model and/or the prediction computational model form a personalized model for said subject.

In a further embodiment the present invention provides a method wherein in addition the expression levels of at least one gene selected from the group comprising AKT1, MYOD1, ACE, PPARGC1A, Elov15 and Sl2a4 g is determined or predicted base on a model of the underlying genetic network and used for said assessment and/or prediction.

In a further embodiment the present invention provides a method wherein samples of at least two consecutive days of said subject are provided and the amount of gene expression is determined and used for said assessment and/or prediction, preferably at least four samples per day.

In a further embodiment the present invention provides a method of predicting the individual diurnal athletic performance time(s) of a subject according to any of claims 1 to 15, wherein each of the time points at which said samples are obtained are at least 2-4 hours apart, and/or wherein the time points span a time period of at least 12 hours of the day, wherein preferably the time points are 4 hours apart, e.g. at 9 h, 13 h, 17 h and 21 h.

In a further embodiment the present invention provides a kit for sampling saliva for use in a method, comprising
sampling tubes for receiving the samples of saliva, wherein each of the sampling tubes contains RNA protect reagent and is configured to enclose one of the samples of saliva to be taken together with the reagent,
wherein preferably each of the sampling tubes is labelled with the time point at which the respective sample is to be taken and/or includes an indication about the amount of saliva for one sample.

In a further embodiment the present invention provides a kit, which further comprises at least one of:
a box,
a cool pack,
at least one form including instructions and/or information about the kit and the method for the subject.

In a further embodiment the present invention provides a kit, wherein the RNA protect reagent is selected from the group comprising EDTA disodium, dihydrate; sodium citrate trisodium salt, dihydrate; ammonium sulfate, powdered; sterile water.

In a further embodiment the present invention provides a kit, wherein said sampling tubes are configured to receive a sample of saliva of 1 mL in addition to 1 mL of the RNA protect reagent, wherein the sampling tubes preferably are at least 2 mL tubes, preferably at least 3 mL tubes, more preferably at least 4 mL tubes, still preferably at least 5 mL tubes.

In a further embodiment the present invention provides a kit, comprising at least six sampling tubes, preferably at least eight sampling tubes.

In a further embodiment the present invention provides a kit for collecting samples of saliva for providing the collected samples of saliva.

### EXAMPLES

In view of the aforementioned explanations, an exemplary embodiment for a general workflow to establish a prediction for the best time for a “behavior B” is outlined below. After that, more specific aspects of the work flow according to an exemplary embodiment for are explained for the “behavior B” being the peak time for sport performance.

1. A Priori Gene Selection:

A set of relevant genes is selected: Core-clock genes, saliva specific oscillating genes (which may show stronger oscillations than the core-clock genes in saliva), and a set of genes that should relate to the behavior B (for sports metabolic genes; for cancer treatment-related genes or drug target genes, etc.). To identify the latter, existing databases are scanned for (1) connections to the core-clock in the genetic network, (2) oscillatory behavior (at least for some tissue, potentially from mice or human), (3) expression level in saliva of healthy human samples or saliva from non-healthy people.

2. Establishment of a Data Set:

Subjects are asked to perform behavior B several times per day, and their performance is recorded. From the same subjects, saliva is sampled for two days 4 times a day.

3. Experimental Analysis:

Gene expression is measured from the saliva samples.

4. Computational Analysis (See Also Description Above):
i. The gene expression data is screen for oscillatory behavior, non-oscillating genes are excluded from the analysis.
ii. The expression data of oscillating genes is used to predict the optimal time for behavior B, as recorded for the subjects. The resulting machine learning algorithm can then be used to predict the optimal time for further subjects for which only saliva data is available.
iii. A core-clock model is supplemented with the genetic network that includes the oscillatory genes identified in step 4i. The model is fitted to the gene expression data.
iv. The model is used to generate data for the prediction of the optimal timing of behavior B. This data has three advantages: 1. The temporal resolution of the data is arbitrarily detailed. 2. In consequence, peak time, phase, amplitude and period can be extracted with more precision. 3. The data includes all genes implemented in the model, which are far more than the genes measured in step 2. With this data as input, step 4ii is repeated, and the performance is compared to the prediction based directly on the data, while accounting for the enhanced potential for overfitting due to a larger amount of processing (more parameters and potentially more data points for the prediction). Although also a simple fit of the data as in step i gives us curves with high temporal resolution, this is not the same as the model fit: A prediction based on the simple fit cannot outperform the prediction from step 4ii, because no information was added. By contrast, the model fit contains information on the biological interaction between the genes (the gene dynamics), which, when added to the prediction, can improve the prediction.
v. Even more important, the model can illuminate the mechanism behind the prediction of step 4ii and iv. This is an important step to enhance trustworthiness of the prediction—the more we understand, the more we can evaluate whether the prediction makes sense (see sports example below: The correlation between the peak times of PER2 and sport performance is likely to explain why PER2 can be used to predict sports performance. Although we have a small sample size of only 10 participants, this gives us confidence that the prediction is not happening by chance, but is really catching some salient feature in the data).
vi. With the idea about the working of the prediction mechanism from step 4v, genes with an even higher expected predictive power can be identified. Those genes can be added to the set from step 1, if the whole workflow is repeated to optimize the prediction even further.

A Posteriori Gene Selection:

Based on the computational analysis, the a priori set of genes is stripped to the genes essential for prediction, in order to minimize the cost of the analysis.

6. Commercial Application:

People provide saliva samples, and the machine learning algorithm resulting from step 4 is used to predict optimal timing of behavior B based on the restricted set of genes from step 5.

According to the present invention, an individual-based (machine learning) prediction of maximal sports performance is provided, wherein individual differences in the amplitude of circadian variation in sports performance are considered. It is shown that a low/high amplitude of ARNTL (BMAL1) gene expression could be used to predict high/low variation in sports performance based on the correlations shown in the results.

The gene expression predicted from the saliva samples can be fitted by a harmonic regression. The fits are done for two core-clock genes, ARNTL (BMAL1) and PER2, and one gene related to sports performance, AKT1. All genes show circadian variation, and AKT1 and ARNTL (BMAL1) show similar dynamics, with the same phase, period, and mean-normalized amplitude, but different overall (mean) expression levels.

Time-course measurements of unstimulated saliva show fluctuations in gene expression across 45 hours are exemplarily shown in FIG. 3. (A) Sampling schemes for saliva collection at 8 time points in two consecutive days (Day 1-9 h, 13 h, 17 h, 21 h; Day 2—as day 1). (B) Time-course RT-qPCR measurements of human saliva normalized to the mean of all time points (ΔΔCT) of ARNTL (BMAL1) (black) and PER2 (grey dashed) of 15 participants (7 female and 8 male) with a fitted linear sine-cosine function. Furthermore, table C shows the harmonic regression analysis and table D provides additional information on the participants and tests performed.

FIG. 4 illustrates that gene expression of ARNTL (BMAL1) and AKT1 covary. (A) Mean-normalized gene expression profile for the three participants for whom the gene AKT1 was measured besides ARNTL (BMAL1) and PER2. The two days were treated as repetitions. The diurnal variation of AKT1 follows ARNTL (BMAL1). (B) The data points from the mean-normalized time-series of ARNTL (BMAL1) and AKT1 correlate, linear regression with p=0.018. (C) Harmonic regression plots for the participants with at least 5 time points. Depicted values are based on individual best fitting period (20 h-28 h). Additionally, the harmonic regression results of AKT1 for the best fitting period are shown in table A.

The analysis used expression levels (2 to the power of ΔCT) of ARNTL (BMAL1) and PER2. In an RT-qPCR assay, a positive reaction is detected by accumulation of a fluorescent signal. But there is also a lot of background fluorescence which needs to be bypassed in order to glean meaningful information from the signal. The cycle threshold (Ct) (alternatively called the quantification cycle (Cq)) is defined as the number of cycles required for the fluorescent signal to cross the threshold (i.e. exceeds background level) which doubles each cycle (1 cycle=2×original sequence abundance, 2 cycles=4×original sequence abundance, etc.). Therefore, Ct levels are inversely proportional to the log 2-normalised amount of target nucleic acid in the sample (i.e. the lower the Ct level, the greater the amount of target nucleic acid in the sample).

The expression level of a gene is dependent on the amount of input RNA or cDNA. In order to get normalised expression values for the gene of interest (the target gene), it is important to choose a suitable gene for use as a reference. A reference gene is a gene whose expression level should not differ between samples, such as a housekeeping or maintenance gene. Comparison of the Ct value of a target gene with that of the reference gene (ΔCT) allows the gene expression level of the target gene to be normalised to the amount of input RNA or cDNA (Overbergh et al, 2003).

The peak time of the gene expression was identified as the time of the day of the maximum of the time series with eight data, i.e. the maximum gene expression over the two recorded days, with the reasoning that errors in the experimental measurement will rather lead to reports of too little than too high abundances.

Participants were separated into two groups that have distinguishable characteristics both on the genetic as well as on the sports level: Inspired by unsupervised clustering algorithms, the saliva data was separated into two groups with high mean ARNTL (BMAL1) expression (>0.04) and low mean ARNTL (BMAL1) expression (<0.04) or early (9 h and 13 h) and late (17 h or 21 h) ARNTL (BMAL1) peak time. For sports and Myoton data, the mean over the repetitions at each timepoint was considered; for the Myoton data the mean was taken over right and left muscles as well as different muscles within two muscle groups, hand muscles (M. adductor pollicis) and leg muscles (M. rectus femoris, M. biceps femoris, M. gastrocnemius). The sports data and the Myoton data was normalized by the mean value over all data points. Measures of standard deviations were compared between the groups with low and high ARNTL (BMAL1), respectively, and statistically significant lower values in one group compared to the other were tested for by a one-tailed Wilcoxon-Mann-Whitney-Test, as implemented in matlab as ranksum( )

The three different uncorrected sample standard deviations were calculated for the sports or Myoton data as: (i) The standard deviation of all data points, including all timepoints and all repetitions. (ii) The standard deviation between different timepoints, where the value for each timepoint results from a mean over the repetitions at this timepoint. (iii) The standard deviation was calculated over the repetitions for each timepoint individually, and then the mean was taken over all timepoints. The latter two measures are meant to separate circadian variations in the data from experimental or physiological noise; the standard deviation between timepoints is likely to be related to daily variations, while the standard deviation of the repetitions rather quantifies measurement noise.

With the aim to predict maximal sports performance from genetic data, we used the python package sklearn for classification. The timing of the maximum for the mean sports performance was labelled as early (9 h or 12 h) or late (15 h or 18 h). Advantageous for classification, the HST resulted in balanced classes with five participants each, while the other tests resulted in unbalanced classes with at least seven participants in the late class. In order to train the machine learning algorithm, participants were separated into a training set (here 9 participants) and a test set (here just one participant). The algorithm is fed with the full data of the training set, and is then tested on the participant of the test set, by feeding it with the genetic data, and comparing the predicted sports timing to the actual sports timing of this participant: if the predicted and actual timing are equal, this is counted as correct prediction.

For predicting early versus late HST performance with machine learning (this is also called a classification), the predictive power of different features of the saliva data was tested: the expression levels of ARNTL (BMAL1) and PER2 (averaged between the two days and normalized by the mean expression), the mean expression levels, the peak times (presented in a one-hot encoding, that means that a peaktime at the first sampling time was presented as 1000, at the second as 0100, at the third as 0010, and at the last as 0001) and the relative expression levels (PER2 divided by ARNTL (BMAL1)). A linear support-vector-machine (SVM, see general section on machine learning) was fitted to predict early or late maximal sports performance based on these features (sklearn.svm.LinearSVCO, the regularization constant C (see general section on machine learning) is set to 1.0 (default of the python implementation)). Using leave-one-subject-out cross-validation (see general section on machine learning), classification performance was evaluated by computing the accuracy, i.e. the number of correct predictions divided by the total number of predictions. For training, the linear SVM is fed with multi-dimensional input data (here e.g. the 8-dimensional mean-normalized gene expression data) and a binary output (early or late sport performance peak). During cross-validation, the training set consists of nine of the ten relevant participants, and the chosen input with p dimensions is denoted as xᵢ∈R^{P}, i∈[1, 2, . . . , 9]. The output yᵢ is encoded as −1 for early sports peak and +1 for a late sports peak, y∈{1,−1} 9. The predicted output for the participant not used in the training set, denoted x₁₀, is then calculated as w^{T}ϕ(x₁₀)+b with the w and b resulting from the minimization, and compared with the correct output y₁₀. Leave-one-subject-out cross-validation implies that this step is repeated 10 times, each time with another participant removed to form the training set. To calculate the accuracy, the number of correct predictions of the left-out subject of the resulting 10 training sets is divided by the number of predictions that were made.

To evaluate the potential power of the circadian molecular profile obtained from saliva to predict sports performance, a pilot analysis was carried out of the 10 participants with both molecular and sports data (5 males, 5 females). Of major interest for athletes is the time of the best sports performance (peak performance time) as well as the amplitude of the daily variation in sports performance.

The analysis suggests that peak performance time is correlated with PER2, as a linear regression can be fitted to the PER2 peak time when plotted against the peak hand-strength test (HST) performance (FIG. 5A, p=0.014). A linear regression fits a linear function to the data, such that the sum of least-squares (the squared distance between function and data point) is minimized. PER2 can also predict early (9 h or 12 h) or late (15 h or 18 h) peak HST performance, compare FIG. 16A. A more precise prediction of the actual peak time was not attempted due to the small sample size. Training a classifier ten times on nine out of ten participants in the context of a leave-one-subject-out cross validation, early or late HST performance could be predicted with an accuracy of up to 100% on the left-out participants. As input to the classifier, exclusively the normalized expression levels of PER2 resulted in a good accuracy of 90% when using individual features). The accuracy could not be improved by using the normalised expression levels of ARNTL (BMAL1), peak times of both genes or relative expression levels as additional feature. Adding the mean ARNTL (BMAL1) levels as additional feature improved the accuracy to 100%. This result is changed when for participant 5 other available saliva data is used, then the prediction accuracy is already at 100% when feeding the algorithm only with the normalized expression levels of PER2. Using as input to the machine learning the peak times of PER2 results in an accuracy of 0.8, however in this case the predictions on the training set showed errors, with one false prediction per training set of nine participants. This shows that, indeed, PER2 peak time is important for the prediction, but that the algorithm uses additional data from the mean-normalised PER2 expression that improves the prediction. Using as individual input normalised expression levels of ARNTL (BMAL1), peak times of ARNTL (BMAL1), relative expression levels or mean ARNTL (BMAL1) levels did not lead to good predictions.

For the participants, the best performance of the day is around 10% higher than the worst performance (FIG. 6B). There were found particularly strong diurnal changes in the HST for participants with an early ARNTL (BMAL1) peak, while small changes occurred for a late ARNTL (BMAL1) peak (ARNTL (BMAL1) level is color-coded in FIG. 5B, black/grey corresponds to early/late ARNTL (BMAL1) peaks as shown in FIG. 5C). To quantify this observation we compared three measures of variation: Based on the mean-normalized HST performance, we calculated the standard deviation (i) for all data, (ii) for the mean values per time point, (iii) for the repetitions at each time point (compare Methods). The standard deviation between timepoints (ii), which relates to the performance changes over the day, is significantly higher for participants with early ARNTL (BMAL1) compared to participants with late BMAL1 (FIG. 5D, p<0.01). The difference is not significant for standard deviation (iii), which rather quantifies measurement noise (FIG. 5D, p=A large performance change over the day is thus predicted by an early BMAL1 peak time, compare FIG. 16B. In addition to the here shown correlation with ARNTL (BMAL1) peak time, the amplitude of the performance changes also correlated with the mean level of ARNTL (BMAL1) expression: Repeating the analysis based on two groups with low (<0.04) and high (>0.04) ARNTL (BMAL1) mean expression levels, there were found significant higher standard deviations for the group with low ARNTL (BMAL1) levels for the mean of HST and CMJ (FIG. 6E left panel, p<0.05), as well as for the muscle tone of the hand muscles (myotonometric data for 3 males, 4 females, FIG. 5E, right panel, p(i)=0.029, p(ii)=0.11, p(iii)=0.029). The results hinted at larger diurnal changes in performance (HST and CMJ) and muscle tone (hand) for participants with low mean ARNTL (BMAL1) levels. This is partly explained by a relation between mean ARNTL (BMAL1) levels and ARNTL (BMAL1) peak time; the group with low mean ARNTL (BMAL1) levels shows significantly earlier ARNTL (BMAL1) peak times (FIG. 5F, Mann-Whitney-U test, p=0.044). No relation was found for the performance of the SRT, for which no repetitions are available, or for the muscle tone of the leg muscles, potentially due to the small sample size of seven participants (FIG. 6).

While the groups with low and high mean ARNTL (BMAL1) levels from above consisted of 3 females, 2 males and 2 females, 3 males, respectively, our data showed a trend for higher ARNTL (BMAL1) levels in males compared to females (FIG. 5G, Welch's t-test, p<0.0001). The gender difference is also visible in the PER2/ARNTL (BMAL1) ratio (FIG. 6H, Welch's t-test, p<all participants with high ratios in Supplementary FIG. 1D are females. In alternative to gender, a grouping based on sports professionalism also results in different BMAL1 expression values, see FIG. 17B. The grouping based on the peak time of ARNTL (BMAL1) does not correlate with the MEQ chronotype (FIG. 51), neither does early and late sport performance, compare also FIG. 17A. An overview of the 1 min warm-up sequence is provided in table F and an overview of detailed statistics is shown in table G.

The analysis suggests that the circadian oscillation of sports performance mainly depends in its amplitude on ARNTL (BMAL1) expression and in its phase (peak performance) on the expression of PER2.

Correlations between molecular rhythms of core-clock genes and athletic performance are shown in FIG. 5. (A) The peak time of PER2 correlates with the time of peak performance of the HST (linear regression with p=0.014). (B) Performance change over the day (max. compared to min.), colour code as in (C). (C) Black and grey groups have an early and late ARNTL (BMAL1) peak time, respectively. (D) Standard deviation calculated on the normalized HST performance for data from different (i) repetitions and time points (p=0.0095), (ii) timepoints (p=0.0095), (iii) repetitions (p=0.057). (E) Separating the groups by the mean expression level of ARNTL (BMAL1) instead of the peak time results in significant differences in the standard deviation of the sports performance of HST and CMJ (left, all p=0.0476) and of the hand muscle frequency (right, p=p=0.11, p=0.0286). (F) Histogram of the time of the day with the highest ARNTL (BMAL1) expression based on the eight saliva samples. Significantly earlier peaks are found for the group with low ARNTL (BMAL1) expression (ranksum, p=0.044). (G) Logarithm of ARNTL (BMAL1) expression levels for all sampling times ordered by male and female participants. Males show a significant higher ARNTL (BMAL1) expression compared to females (Welch's t-test, p<(H) Logarithm of the ratio of PER2 and ARNTL (BMAL1) expression levels for all sampling times ordered by male and female participants. Females show a significant higher ARNTL (BMAL1) expression compared to males (Welch's t-test, p<0.0001). (I) Early or late ARNTL (BMAL1) peaks occur in any of the three investigated MEQ chronotype.

FIG. 6 shows diagrams of standard deviations of normalized sports and muscle tone data (L: group with low ARNTL (BMAL1), H: group with high ARNTL (BMAL1)). Mean standard deviation calculated on the normalized sports performance and the normalized muscle tone data for different (i) repetitions and timepoints, (ii) timepoints, (iii) repetitions (for details see Methods). (A) HST, (B) CMJ, (C) SRT (no repetitions were measured, thus the standard deviation (i) over all data is the same as (ii) over timepoints), (D) muscle tone of the leg muscles (M. rectus femoris, M. biceps femoris, M. gastrocnemius). The performed 15 min warm-up sequence is depicted in table F an overview of detailed statistics is shown in table G.

FIG. 18 exemplifies for one subject how a circadian profile including gene expression data (FIG. 18A) can be used to predict best exercise performance, both for strength exercises and endurance exercises (FIG. 18B). FIG. 19 exemplifies for another subject that the prediction based on gene expression profiles is fitted by the circadian variation in sports performance, both for strength exercises and endurance exercises.

In one embodiment for an application of the methods and the model of the present invention, light therapy is implemented as a 5-fold increase in PER2 maximal transcription rate and a 5-fold decrease in PER2 degradation rate. Light therapy 1 h after wakeup leads to no changes in the phase, or, for longer duration, to a small phase advance of 6 minutes, see FIG. 26. Light 8 h after wakeup leads to half an hour of delay for one-hour treatment, and a bit more than an hour for two-hour treatment. Strongest responses occur for light therapy starting 14 h after wakeup, inducing delays of up to 5 h, see FIG. 26.

The present methods may be used for guidance of light therapy.

Light therapy can be used to enhance the oscillations, so if the clock is not very robust, the person might feel more tired for e.g., with light therapy one could address that. The experimental kit and mathematical model according to the present invention can also be used to show how the circadian profile of a patient looks like or any person) and then if we detect problems in the circadian profile, the model can helps to decide on the best times to apply certain therapies to induce the clock (e.g. light), to make the clock more robust, this would have immediate implications on the overall well-being, for e.g. better sleep rhythms.

If patients get their rhythms more robust, than it is possible to also better determine the time for treatment. If a patient has a very flat clock (no oscillations) it would make sense to do light therapy to enhance the clock and then to determine the best time to treat. The present model can also be used to enhance the clock (applying for e.g. light), this will also contribute for the overall well-being of the patient.

With reference to FIG. 7, modeling the genetic network associated with sports performance is explained. The model simulates gene expression of the core-clock genes and clock-regulated genes via two interconnected feedback loops (Per/Cry loop and Rev-Erb/Ror/Bmal loop). The model parameters were fitted to the measured data of gene expression. The model predicts the rhythmicity of athletic performance based on the oscillatory behaviour of Ace and Ppargcla genes.

In the genetic network for sports performance extension of the core-clock illustrated in FIG. 7, the plots show the gene expression of 2 core-clock genes and 4 clock-regulated genes crucial for athletic performance and metabolism. Dots indicate the measured gene expression of Arntl (Bmal1), Per2, Ace, and Ppargcla. Solid lines represent the in-silico gene expression generated with the mathematical model, which was fitted to the experimental data of the previously mentioned genes. The model additionally predicts the expression of Elovl5 and Sl2a4 g genes, important for metabolism.

An example of predicting the peak time for sport performance is described with reference to FIGS. 8a to 8c, in which FIG. 8a illustrates the core-clock genes. Genes important for athletic performance and metabolism are illustrated in FIGS. 8b and 8c.

The result is illustrated in FIG. 9, where the mathematical model computes the athletic performance based on the expression of Ppargcla and Ace genes. Accordingly, the predicted time window for maximum athletic performance is 11:00-15:00 hours, the peak of athletic performance occurs 5 hours since awakening, and the recommended time-window for meals is 08:00-18:00 hours.

FIG. 10 illustrates ARNTL (BMAL1) and PER2 expression display variation during the day in human blood, hair and saliva samples. (A) Three time-point comparison of ARNTL (BMAL1) and PER2 expression for the averaged data of all Participants in FIG. 1. Expression data is compared to the first time-point (Early). For hair and saliva data Early, Middle and Late time-points represent 9 h, 17 h and 21 h, respectively. For PBMCs data Early, Middle and Late time-points represent 10 h, 16 h and 19 h, respectively. Depicted are mean+SEM. (B) Time-course RT-qPCR measurements normalised to the mean of all time points (ΔΔCT) of ARNTL (BMAL1) and PER2 of Participant 1, 2, and 13 with a fitted linear sine-cosine function (period=24 h). For Participant 1, we collected one additional sample at 21 h on the 2nd day. Harmonic regression best p-values for tested periods (20-28 h): Participant 1; BMAL1 (0.517, period=21.4 h), PER2 (0.353, period=24.0 h). Participant 2; ARNTL (BMAL1) (0.038, period=20.0 h), PER2 (0.276, period=28.0 h). Participant 13; ARNTL (BMAL1) (0.014, period=20 h), PER2 (0.086, period=21.4 h). (C) Time-course RT-qPCR measurements of human PBMCs normalised to the mean of all time points (ΔΔCT) of ARNTL (BMAL1), CLOCK, NPAS2, PER2, CRY2, NR1D1, and RORB of Participant 2 and 5 with a fitted linear sine-cosine function (period=24 h). Harmonic regression best p-values: Participant 2; ARNTL (BMAL1) (3.05 E-01, period=20 h), CLOCK (6.31 E-02, period=28 h), NPAS2 (1.67 E-01, period=20 h), PER2 (4.78 E-04, period=20.8 h), CRY2 (7.17 E-01, period=20 h), NR1D1 (1.48 E-01, period=28 h) and RORB (7.58 E-01, period=20 h). Participant 5; ARNTL (BMAL1) (5.56 E-01, period=20 h), CLOCK (6.81 E-01, period=28 h), NPAS2 (9.75 E-02, period=28 h, PER2 (1.23 E-01, period=28 h), CRY2 (5.40 E-01, period=28 h), NR1D1 (6.43 E-01, period=28 h) and RORB (7.73 E-01, period=28 h). (D) Average PER2 expression compared to ARNTL (BMAL1) using saliva time-course data for each participant (mean+SEM).

FIG. 11 illustrates HST base line measurements. Depicted are mean values for three participants (9 h-18 h in one-hour intervals, N=3, mean±SEM). The HST measurements were randomly distributed across three measurement days with one day break in between. The red full circles represent the time point chosen for the subsequent training sessions. For detailed HST base line measurements see table E.

FIG. 12 illustrates Myotonometric analysis shows daily variation in muscle tone (frequency, F) for female and male participants. Only participants who completed all training sessions were included in the MyotonPRO measurements (N=12). Mean of normalized scores for the myotonometric parameter frequency [Hz] for each training session (T1-9 h, T2-12 h, T3-15 h, T4-18 h) and each muscle: M. Deltoideus, M. Triceps Brachii, M. adductor pollicis, M. rectus femoris, M. biceps femoris, M. gastrocnemius. The measurements were carried out from top to bottom on the right (Right bar) and the corresponding left (Left bar) side of the body. Corresponding statistics for intrapersonal variation between each time points can be found in Supplementary Table B. *p<0.05, compared to time point 9 h. A detailed statistical analysis is depicted in table B and H. FIG. 13 illustrates an optimized ratio between collected saliva and RNA stabilization reagent, which yealds the best RNA concentration. FIGS. 14 and 15 illustrate the saliva RNA concentration measured over time with an optimized ratio determined in FIG. 13 (1:1 with 1.5 mL saliva) and the expression of core clock genes in these samples.

Tables

TABLE A
Harmonic regression results of AKT1 for
the best fitting period in FIG. 4.

| Participant | qvals | pvals | Acrophase [h] | amplitude | Period [h] |
|---|---|---|---|---|---|
| Participant 3 | 0.249 | 0.178 | 18 | 1.506 | 28 |
| Participant 5 | 0.061 | 0.017 | 12 | 1.042 | 28 |
| Participant 6 | 0.011 | 0.001 | 11 | 1.068 | 26.6 |
| Participant 12 | 0.696 | 0.229 | 14 | 1.067 | 20 |
| Participant 21 | 0.249 | 0.167 | 14 | 1.386 | 28 |

TABLE B
Statistical analysis for FIG. 12, pairwise
comparisons (Friedmann test).

| | T1 | T2 | T3 | T4 |
|---|---|---|---|---|
| M. Deltoideus_Left (Females) | | | | |
| T1 | NA | | | |
| T2 | 0.038 | NA | | |
| T3 | 0.038 | 1 | NA | |
| T4 | 0.047 | 0.902 | 0.902 | NA |
| M. Deltoideus_Right (Females) | | | | |
| T1 | NA | | | |
| T2 | 0.629 | NA | | |
| T3 | 0.629 | 0.797 | NA | |
| T4 | 0.797 | 0.670 | 0.629 | NA |
| M. Triceps Brachii_Left (Females) | | | | |
| T1 | NA | | | |
| T2 | 0.807 | NA | | |
| T3 | 0.807 | 0.807 | NA | |
| T4 | 0.807 | 0.807 | 0.807 | NA |
| M. Triceps Brachii_Right(Females) | | | | |
| T1 | NA | | | |
| T2 | 0.741 | NA | | |
| T3 | 0.741 | 0.741 | NA | |
| T4 | 1 | 0.741 | 0.741 | NA |
| M. Rectus Femoris_Left(Females) | | | | |
| T1 | NA | | | |
| T2 | 0.922 | NA | | |
| T3 | 0.922 | 0.964 | NA | |
| T4 | 0.922 | 1 | 0.964 | NA |
| M. Rectus Femoris_Right(Females) | | | | |
| T1 | NA | | | |
| T2 | 0.210 | NA | | |
| T3 | 0.434 | 0.539 | NA | |
| T4 | 0.049 | 0.210 | 0.142 | NA |
| M. Biceps Femoris_Left(Females) | | | | |
| T1 | NA | | | |
| T2 | 0.441 | NA | | |
| T3 | 0.377 | 0.712 | NA | |
| T4 | 1 | 0.441 | 0.377 | NA |
| M. Biceps Femoris_Right(Females) | | | | |
| T1 | NA | | | |
| T2 | 0.516 | NA | | |
| T3 | 0.516 | 0.488 | NA | |
| T4 | 0.790 | 0.516 | 0.516 | NA |
| M. Adductor Pollicis_Left(Females) | | | | |
| T1 | NA | | | |
| T2 | 0.797 | NA | | |
| T3 | 0.670 | 0.629 | NA | |
| T4 | 0.629 | 0.629 | 0.797 | NA |
| M. Adductor Pollicis_Right(Females) | | | | |
| T1 | NA | | | |
| T2 | 0.473 | NA | | |
| T3 | 0.185 | 0.333 | NA | |
| T4 | 0.185 | 0.392 | 0.773 | NA |
| M. Gastrocnemius_Left(Females) | | | | |
| T1 | NA | | | |
| T2 | 0.263 | NA | | |
| T3 | 0.197 | 0.038 | NA | |
| T4 | 0.197 | 0.038 | 1 | NA |
| M. Gastrocnemius_Right(Females) | | | | |
| T1 | NA | | | |
| T2 | 0.382 | NA | | |
| T3 | 0.589 | 0.382 | NA | |
| T4 | 0.382 | 0.589 | 0.589 | NA |
| M. Deltoideus_Left (Males) | | | | |
| T1 | NA | | | |
| T2 | 0.963 | NA | | |
| T3 | 0.963 | 0.963 | NA | |
| T4 | 0.963 | 0.963 | 1 | NA |
| M. Deltoideus_Right (Males) | | | | |
| T1 | NA | | | |
| T2 | 0.481 | NA | | |
| T3 | 0.600 | 0.719 | NA | |
| T4 | 0.601 | 0.650 | 0.792 | NA |
| M. Triceps Brachii_Left (Males) | | | | |
| T1 | NA | | | |
| T2 | 0.458 | NA | | |
| T3 | 0.466 | 0.689 | NA | |
| T4 | 0.534 | 0.534 | 0.689 | NA |
| M. Triceps Brachii_Right (Males) | | | | |
| T1 | NA | | | |
| T2 | 0.037 | NA | | |
| T3 | 0.203 | 0.203 | NA | |
| T4 | 0.203 | 0.203 | 1 | NA |
| M. Rectus Femoris_Left(Males) | | | | |
| T1 | NA | | | |
| T2 | 0.652 | NA | | |
| T3 | 0.039 | 0.047 | NA | |
| T4 | 0.219 | 0.129 | 0.360 | NA |
| M. Rectus Femoris_Right(Males) | | | | |
| T1 | NA | | | |
| T2 | 0.203 | NA | | |
| T3 | 0.203 | 0.067 | NA | |
| T4 | 1 | 0.203 | 0.203 | NA |
| M. Biceps Femoris_Left(Males) | | | | |
| T1 | NA | | | |
| T2 | 0.637 | NA | | |
| T3 | 1 | 0.637 | NA | |
| T4 | 1 | 0.637 | 1 | NA |
| M. Biceps Femoris_Right(Males) | | | | |
| T1 | NA | | | |
| T2 | 0.798 | NA | | |
| T3 | 0.798 | 0.798 | NA | |
| T4 | 0.798 | 0.798 | 0.798 | NA |
| M. Adductor Pollicis_Left(Males) | | | | |
| T1 | NA | | | |
| T2 | 1 | NA | | |
| T3 | 0.801 | 0.801 | NA | |
| T4 | 0.076 | 0.076 | 0.118 | NA |
| M. Adductor Pollicis_Right(Males) | | | | |
| T1 | NA | | | |
| T2 | 0.605 | NA | | |
| T3 | 0.605 | 0.605 | NA | |
| T4 | 0.605 | 0.605 | 0.605 | NA |
| M. Gastrocnemius_Left(Males) | | | | |
| T1 | NA | | | |
| T2 | 0.228 | NA | | |
| T3 | 0.584 | 0.228 | NA | |
| T4 | 0.228 | 0.584 | 0.421 | NA |
| M. Gastrocnemius_Right(Males) | | | | |
| T1 | NA | | | |
| T2 | 1 | NA | | |
| T3 | 0.740 | 0.740 | NA | |
| T4 | 0.740 | 0.740 | 0.740 | NA |

TABLE C
Harmonic regression analysis (see FIG. 3).

| Mesor | qvals | pvals | Acrophase [h] | Acrophase [radians] | Amplitude | Period [h] | Condition |
|---|---|---|---|---|---|---|---|
| −0.50 | 0.49 | 0.18 | 15.83 | 4.14 | 1.22 | 23.6 | BMAL1_Participant 5 |
| 0.42 | 0.60 | 0.42 | 25.32 | 6.62 | 0.92 | 26.2 | PER2_Participant 5 |
| −0.12 | 0.24 | 0.01 | 6.67 | 1.74 | 1.80 | 28 | BMAL1_Participant 15 |
| −0.03 | 0.70 | 0.54 | 6.60 | 1.72 | 0.58 | 28 | PER2_Participant 15 |
| −0.03 | 0.31 | 0.05 | 0.59 | 0.15 | 1.49 | 20.9 | BMAL1_Participant 21 |
| 0.29 | 0.35 | 0.06 | 6.30 | 1.70 | 1.48 | 20 | PER2_Participant 21 |
| −2.03 | 0.52 | 0.23 | 15.08 | 3.94 | 4.38 | 25.2 | BMAL1_Participant 8 |
| −0.63 | 0.70 | 0.58 | 12.54 | 3.28 | 1.26 | 28 | PER2_Participant 8 |
| −0.71 | 0.21 | 0.01 | 18.33 | 4.79 | 3.85 | 22.9 | BMAL1_Participant 9 |
| −0.06 | 0.51 | 0.15 | 3.29 | 0.86 | 2.33 | 20 | PER2_Participant 9 |
| 0.02 | 0.15 | 0.01 | 20.33 | 5.32 | 1.78 | 20.8 | BMAL1_Participant 17 |
| 0.38 | 0.70 | 0.40 | 0.51 | 0.13 | 0.72 | 28 | PER2_Participant 17 |
| −0.01 | 0.48 | 0.14 | 21.05 | 5.51 | 0.28 | 22.1 | BMAL1_Participant 13 |
| 0.05 | 0.56 | 0.15 | 24.33 | 6.36 | 0.11 | 28 | PER2_Participant 13 |
| −0.16 | 0.70 | 0.54 | 17.04 | 4.46 | 0.42 | 28 | PER2_Participant 11 |
| −0.16 | 0.73 | 0.63 | 15.67 | 4.10 | 0.34 | 28 | BMAL1_Participant 11 |
| −1.47 | 0.45 | 0.06 | 14.34 | 3.75 | 3.50 | 26.6 | BMAL1_Participant 1 |
| 0.29 | 0.31 | 0.02 | 21.82 | 5.71 | 1.71 | 26.9 | PER2_Participant 1 |
| −0.85 | 0.21 | 0.02 | 16.80 | 4.39 | 2.65 | 23.5 | BMAL1_Participant 3 |
| −0.61 | 0.21 | 0.01 | 13.75 | 3.59 | 1.63 | 22.6 | PER2_Participant 3 |
| −0.12 | 0.10 | 0.03 | 13.36 | 3.49 | 1.43 | 20 | BMAL1_Participant 19 |
| 0.38 | 0.56 | 0.07 | 27.44 | 7.18 | 0.76 | 28 | PER2_Participant 19 |
| 0.17 | 0.88 | 0.58 | 4.98 | 1.30 | 1.07 | 20 | BMAL1_Participant 2 |
| 0.02 | 0.69 | 0.29 | 11.28 | 2.95 | 0.50 | 20.3 | PER2_Participant 2 |
| 0.00 | 0.60 | 0.31 | 21.15 | 5.53 | 0.56 | 23.4 | BMAL1_Participant 4 |
| −0.04 | 0.63 | 0.23 | 20.07 | 5.25 | 0.28 | 20.7 | PER2_Participant 4 |
| −0.01 | 0.67 | 0.28 | 1.88 | 0.49 | 0.71 | 20 | BMAL1_Participant 12 |
| −0.13 | 0.84 | 0.53 | 18.94 | 4.95 | 0.76 | 20 | PER2_Participant 12 |
| −0.60 | 0.46 | 0.09 | 12.22 | 3.19 | 1.46 | 26.7 | BMAL1_Participant 6 |
| −0.05 | 0.40 | 0.09 | 12.83 | 3.35 | 0.42 | 22 | PER2_Participant 6 |

TABLE D
List of participants and tests (MEQ, Sports tests, Molecular tests, Myotonometry)
performed (see FIG. 3). Y = Yes, participant has carried out the test.

| | | | sports tests | | | molecular tests | | | myotonometry |
|---|---|---|---|---|---|---|---|---|---|
| Participant # | gender | MEQ | # training sessions | # round of tests | HST_long | saliva | hair | blood | MyotonPRO |
| 1 | male | intermediate | — | — | — | Y | Y | — | — |
| 2 | male | intermediate | — | — | — | Y | Y | Y | — |
| 3 | female | intermediate | — | — | Y | Y | — | — | — |
| 4 | male | moderate morning | — | — | Y | Y | Y | — | — |
| 5 | female | moderate morning | 4 | 1 | Y | Y | — | Y | Y |
| 6 | female | moderate evening | 4 | 1 | — | Y | — | — | Y |
| 7 | female | intermediate | 4 | 1 | — | — | — | — | Y |
| 8 | female | intermediate | 4 | 1 | — | Y | — | — | Y |
| 9 | female | moderate evening | 4 | 1 | — | Y | — | — | Y |
| 10 | male | intermediate | 4 | 1 | — | — | — | — | Y |
| 11 | male | intermediate | 4 | 1 | — | Y | — | — | Y |
| 12 | female | intermediate | — | — | — | Y | — | — | — |
| 13 | male | intermediate | 4 | 1 | — | Y | Y | — | Y |
| 14 | male | intermediate | 4 | 1 | — | — | — | — | Y |
| 15 | male | intermediate | 4 | 1 | — | Y | — | — | Y |
| 16 | male | moderate evening | 4 | 1 | — | — | — | — | Y |
| 5 | female | moderate morning | 3 | 2 | — | — | — | — | — |
| 8 | female | intermediate | 3 | 2 | — | — | — | — | — |
| 17 | female | moderate evening | 4 | 2 | — | Y | — | — | — |
| 18 | male | intermediate | 3 | 2 | — | — | — | — | — |
| 10 | male | intermediate | 4 | 2 | — | — | — | — | — |
| 19 | male | moderate morning | 3 | 2 | — | Y | — | — | — |
| 20 | male | moderate evening | 4 | 2 | — | — | — | — | — |
| 21 | male | moderate morning | 4 | 2 | — | Y | — | — | — |

TABLE E
HST base line measurements (see FIG. 11) (9 h-18 h in
one hour intervals, N = 3, mean ± SEM)

| Time [h] | 9 | 10 | 11 | 12 | 13 | 14 | 15 | 16 | 17 | 18 |
|---|---|---|---|---|---|---|---|---|---|---|
| mean | 0.86 | 1.03 | 0.98 | 0.97 | 0.97 | 1.05 | 1.08 | 1.08 | 0.97 | 1.03 |
| SEM | 0.05 | 0.03 | 0.02 | 0.02 | 0.02 | 0.05 | 0.02 | 0.03 | 0.03 | 0.08 |

TABLE F
15 min warm-up sequence carried out before the exercises: HST, CMJ, SRT (see FIGS. 5 and 6).

| Exercise | Repetitions | Aim and muscle group used |
|---|---|---|
| Jogging - Forward and backwards | 2 × 20 m Forwards | whole body warm up |
| | 2 × 20 m Backwards | |
| “Butt kicks” | 2 × 20 m | ischiocrucal muscles |
| High knees | 2 × 20 m | lower limbs |
| Sidesteps | 1 × 20 m | abductors |
| Cross-step exercise | 1 × 20 m | foot coordination |
| Knee to chest | 10× | stretching of the hamstrings (M. biceps femoris) |
| Foot inside pull | 10× | stretching of the leg adductors |
| Lunge | 10× | stretching of M. psoas major, activating the leg muscles and stability work |
| Caterpillar | 5× | stretching the hamstrings (M. biceps femoris), core muscles activation and |
| | | activation for the muscles of the upper limbs |
| Jogging - Forward and backwards | 1 × 20 m Forwards | whole body warm up before sprinting |
| | 1 × 20 m Backwards | |
| Intensity Sprints | 30% max speed | step by step preparation for explosive workout |
| | 60% max speed | |
| | 90% max speed | |
| | 20 m sprint max speed | |

TABLE G
Statistics corresponding to FIGS. 5 & 6. Friedmann
test was used for determining the pairwise intrapersonal
variations between different training times.

| Test | Gender | Time point | T1 | T2 | T3 | T4 |
|---|---|---|---|---|---|---|
| HST | Male | T1 | NA | | | |
| HST | Male | T2 | 0.879 | NA | | |
| HST | Male | T3 | 0.879 | 1 | NA | |
| HST | Male | T4 | 0.879 | 1 | 1 | NA |
| HST | Female | T1 | NA | | | |
| HST | Female | T2 | 0.373 | NA | | |
| HST | Female | T3 | 0.373 | 0.789 | NA | |
| HST | Female | T4 | 0.514 | 0.514 | 0.514 | NA |
| CMJ | Male | T1 | NA | | | |
| CMJ | Male | T2 | 0.681 | NA | | |
| CMJ | Male | T3 | 0.015 | 0.005 | NA | |
| CMJ | Male | T4 | 0.681 | 1 | 0.005 | NA |
| CMJ | Female | T1 | NA | | | |
| CMJ | Female | T2 | 0.805 | NA | | |
| CMJ | Female | T3 | 0.805 | 1 | NA | |
| CMJ | Female | T4 | 0.622 | 0.805 | 0.805 | NA |
| SRT | Male | T1 | NA | | | |
| SRT | Male | T2 | 0.017 | NA | | |
| SRT | Male | T3 | 0.034 | 0.550 | NA | |
| SRT | Male | T4 | 0.0003 | 0.098 | 0.034 | NA |
| SRT | Female | T1 | NA | | | |
| SRT | Female | T2 | 0.481 | NA | | |
| SRT | Female | T3 | 0.481 | 0.893 | NA | |
| SRT | Female | T4 | 0.481 | 0.827 | 0.827 | NA |

TABLE H
Statistical analysis with Friedmann test corresponding to FIG. 12.

| Muscle_Name | | T1 | T2 | T3 | T4 |
|---|---|---|---|---|---|
| m_deltoideus_L | T1 | NA | | | |
| m_deltoideus_L | T2 | 0.131 | NA | | |
| m_deltoideus_L | T3 | 0.060 | 0.629 | NA | |
| m_deltoideus_L | T4 | 0.060 | 0.707 | 0.786 | NA |
| m_deltoideus_R | T1 | NA | | | |
| m_deltoideus_R | T2 | 0.655 | NA | | |
| m_deltoideus_R | T3 | 1 | 0.655 | NA | |
| m_deltoideus_R | T4 | 0.655 | 1 | 0.655 | NA |
| m_triceps_brachii_L | T1 | | | | |
| m_triceps_brachii_L | T2 | 0.718 | NA | | |
| m_triceps_brachii_L | T3 | 0.718 | 0.766 | NA | |
| m_triceps_brachii_L | T4 | 0.718 | 0.792 | 0.792 | NA |
| m_triceps_brachii_R | T1 | NA | | | |
| m_triceps_brachii_R | T2 | 0.585 | NA | | |
| m_triceps_brachii_R | T3 | 0.649 | 0.649 | NA | |
| m_triceps_brachii_R | T4 | 0.585 | 0.792 | 0.720 | NA |
| m_add_pollicis_L | T1 | NA | | | |
| m_add_pollicis_L | T2 | 0.674 | NA | | |
| m_add_pollicis_L | T3 | 0.008 | 0.012 | NA | |
| m_add_pollicis_L | T4 | 0.062 | 0.113 | 0.255 | NA |
| m_add_pollicis_R | T1 | NA | | | |
| m_add_pollicis_R | T2 | 1 | NA | | |
| m_add_pollicis_R | T3 | 0.051 | 0.051 | NA | |
| m_add_pollicis_R | T4 | 0.028 | 0.028 | 0.699 | NA |
| m_rectus_femoris_L | T1 | NA | | | |
| m_rectus_femoris_L | T2 | 1 | NA | | |
| m_rectus_femoris_L | T3 | 0.241 | 0.241 | NA | |
| m_rectus_femoris_L | T4 | 1 | 1 | 0.241 | NA |
| m_rectus_femoris_R | T1 | NA | | | |
| m_rectus_femoris_R | T2 | 0.127 | NA | | |
| m_rectus_femoris_R | T3 | 0.214 | 0.014 | NA | |
| m_rectus_femoris_R | T4 | 1 | 0.127 | 0.214 | NA |
| m_biceps_femoris_L | T1 | NA | | | |
| m_biceps_femoris_L | T2 | 0.777 | NA | | |
| m_biceps_femoris_L | T3 | 0.250 | 0.190 | NA | |
| m_biceps_femoris_L | T4 | 0.003 | 0.003 | 0.059 | NA |
| m_biceps_femoris_R | T1 | NA | | | |
| m_biceps_femoris_R | T2 | 0.780 | NA | | |
| m_biceps_femoris_R | T3 | 0.017 | 0.025 | NA | |
| m_biceps_femoris_R | T4 | 0.017 | 0.017 | 0.780 | NA |
| m_gastr_cm_L | T1 | NA | | | |
| m_gastr_cm_L | T2 | 0.005 | NA | | |
| m_gastr_cm_L | T3 | 0.474 | 0.0009 | NA | |
| m_gastr_cm_L | T4 | 0.775 | 0.007 | 0.388 | NA |
| m_gastr_cm_R | T1 | NA | | | |
| m_gastr_cm_R | T2 | 0.436 | NA | | |
| m_gastr_cm_R | T3 | 0.518 | 0.518 | NA | |
| m_gastr_cm_R | T4 | 0.518 | 0.518 | 0.792 | NA |

1. Method of assessing the circadian rhythm or circadian profile of a subject and/or assessing and predicting the athletic performance of said subject, wherein said method comprises the steps of:
Providing at least three samples of saliva, more preferably four samples of saliva, from said subject, wherein said samples have been taken at different time points over the day,
Determining gene expression of at least two members of genes for the core-clock network, in particular of at least two members of the group comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, in each of said samples, and
Assessing and predicting by means of a computational step based on said expression levels of at least two members of the groups comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, over the day the circadian rhythm of said subject and/or the individual diurnal athletic performance times, both for strength exercises and endurance exercises.

2. Method according to claim 1, wherein gene expression is determined using a method selected from quantitative PCR (RT-qPCR), NanoString, sequencing and microarray.

3. Method according to claim 1, wherein assessing the circadian rhythm of said subject comprises determining a periodic function for each of at least two members of the groups comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, that approximates said expression levels for each of at least two members of the groups comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, preferably comprising curve fitting of a non-linear periodic model function to the respective expression levels, wherein the curve fitting is preferably carried out by means of harmonic regression.

4. Method according to claim 1, wherein the computational step comprises
processing the determined expression levels and/or the respectively fitted periodic functions to derive characteristic data for each of at least two members of the groups comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, said processing comprising determining the mean expression level of expression of at least two members of the groups comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, and normalizing the expression levels using the mean expression level.

5. Method according to claim 4, wherein said characteristic data comprise:
the amplitude of change of expression of a gene, and/or the amplitude relative to one of the other genes, and/or
the mean expression level of expression of a gene, and/or and/or the mean relative to one of the other genes, and/or
the peak expression level of a gene, and/or the peak relative to one of the other genes, and/or
the amplitude of change of expression of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC over the day, and/or
the relative difference of the amplitudes of change of expression of any two of ARNTL (BMAL1) and/or ARNTL2 and/or CLOCK, and/or NPAS2 and/or PER1 and/or PER2 and/or PER3 and/or CRY1 and/or CRY2 and/or NR and/or NR and/or RORA and/or RORB and/or RORC, and/or
the mean expression level of expression of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC, and/or
the relative difference of the mean expression levels of expression of any two of ARNTL (BMAL1) and/or ARNTL2 and/or CLOCK, and/or NPAS2 and/or PER1 and/or PER2 and/or PER3 and/or CRY1 and/or CRY2 and/or NR and/or NR and/or RORA and/or RORB and/or RORC, and/or
the peak expression level of ARNTL (BMAL1) and/or ARNTL2 and/or CLOCK, and/or NPAS2 and/or PER1 and/or PER2 and/or PER3 and/or CRY1 and/or CRY2 and/or NR and/or NR1D2 and/or RORA and/or RORB and/or RORC over the day, and/or
the relative difference of the peak expression levels of any two of ARNTL (BMAL1) and/or ARNTL2 and/or CLOCK, and/or NPAS2 and/or PER1 and/or PER2 and/or PER3 and/or CRY1 and/or CRY2 and/or NR and/or NR and/or RORA and/or RORB and/or RORC, and/or
the time of the peak expression level of ARNTL (BMAL1) and/or ARNTL2 and/or CLOCK, and/or NPAS2 and/or PER1 and/or PER2 and/or PER3 and/or CRY1 and/or CRY2 and/or NR1D1 and/or NR1D2 and/or RORA and/or RORB and/or RORC,
the relative difference of the times of the peak expression level of any two of ARNTL (BMAL1) and/or ARNTL2 and/or CLOCK, and/or NPAS2 and/or PER1 and/or PER2 and/or PER3 and/or CRY1 and/or CRY2 and/or NR and/or NR and/or RORA and/or RORB and/or RORC,
wherein the amplitude, period and phase expression level of expression of ARNTL (BMAL1) and/or ARNTL2 and/or CLOCK, and/or NPAS2 and/or PER1 and/or PER2 and/or PER3 and/or CRY1 and/or CRY2 and/or NR and/or NR and/or RORA and/or RORB and/or RORC are extracted from the determined expression levels and/or the respectively fitted periodic function.

6. Method according to claim 1, wherein the computational step further comprises
fitting a network computational model to the derived characteristic data that comprises a representation of the periodic time course of the expression levels for each of at least two members of the group comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2, as well as a representation of the periodic time course of the expression level for at least one, preferably a plurality of further gene(s) included in a gene regulatory network that includes said at least two members the group comprising ARNTL (BMAL1), ARNTL2, CLOCK, PER1, PER2, PER3, NPAS2, CRY1, CRY2, NR1D1, NR1D2, RORA, RORB, RORC, in particular ARNTL (BMAL1) and PER2; and/or
training a machine learning algorithm on the derived characteristic data of the network computational model, particularly optimize in terms of the representation of the periodic time course of the expression level for the at least one further gene.

7. Method according to claim 4, wherein assessing and/or predicting the individual diurnal athletic performance times comprises in the computational step
fitting a prediction computational model on data obtained from said fitted periodic functions and/or said network computational model, wherein the prediction computational model is based on machine learning, including at least one classification method and/or at least one clustering method wherein said method(s) are preferably selected from the group comprising:
K-nearest neighbor algorithm, unsupervised clustering, deep neural networks, random forest algorithm, and support vector machines.

8. Method according to claim 1, wherein the network computational model and/or the prediction computational model form a personalized model for said subject.

9. Method according to claim 1, wherein in addition the expression levels of at least one gene selected from the group comprising AKT1, MYOD1, ACE, PPARGC1A, Elov15 and Sl2a4 g is determined or predicted base on a model of the underlying genetic network and used for said assessment and/or prediction.

10. Method of predicting the individual diurnal athletic performance time(s) of a subject according to claim 1, wherein each of the time points at which said samples are obtained are at least 2-4 hours apart, and/or wherein the time points span a time period of at least 12 hours of the day, wherein preferably the time points are 4 hours apart, e.g. at 9 h, 13 h, 17 h and 21 h.

11. Kit for sampling saliva for use in a method according to claim 1, comprising
sampling tubes for receiving the samples of saliva, wherein each of the sampling tubes contains RNA protect reagent and is configured to enclose one of the samples of saliva to be taken together with the reagent,
wherein preferably each of the sampling tubes is labelled with the time point at which the respective sample is to be taken and/or includes an indication about the amount of saliva for one sample.

12. Kit according to claim 11, further comprising at least one of:
a box,
a cool pack,
at least one form including instructions and/or information about the kit and the method for the subject.

13. Kit according to claim 11, wherein the RNA protect reagent is selected from the group comprising EDTA disodium, dihydrate; sodium citrate trisodium salt, dihydrate; ammonium sulfate, powdered; sterile water.

14. Kit according to claim 11, wherein said sampling tubes are configured to receive a sample of saliva of 1 mL in addition to 1 mL of the RNA protect reagent, wherein the sampling tubes preferably are at least 2 mL tubes, preferably at least 3 mL tubes, more preferably at least 4 mL tubes, still preferably at least 5 mL tubes.

15. A method for collecting samples of saliva for providing the collected samples of saliva, said method being performed by a kit of claim 11.


SEQUENCE LISTING (20 sequences)

SEQ ID NO: 1 - DNA, 1878 bp, Human
atggcagaccagagaatggacatttcttcaaccatcagtgatttcatgtccccgggccccaccgacctgctttccagctctcttggtaccagtggtgtggattgcaaccgcaaacggaaaggcagctccactgactaccaagaaagcatggacacagacaaagatgaccctcatggaaggttagaatatacagaacaccaaggaaggataaaaaatgcaagggaagctcacagtcagattgaaaagcggcgtcgggataaaatgaacagttttatagatgaattggcttctttggtaccaacatgcaacgcaatgtccaggaaattagataaacttactgtgctaaggatggctgttcagcacatgaaaacattaagaggtgccaccaatccatacacagaagcaaactacaaaccaacttttctatcagacgatgaattgaaacacctcattctcagggcagcagatggatttttgtttgtcgtaggatgtgaccgagggaagatactctttgtctcagagtctgtcttcaagatcctcaactacagccagaatgatctgattggtcagagtttgtttgactacctgcatcctaaagatattgccaaagtcaaggagcagctctcctcctctgacaccgcaccccgggagcggctcatagatgcaaaaactggacttccagttaaaacagatataacccctgggccatctcgattatgttctggagcacgacgttctttcttctgtaggatgaagtgtaacaggccttcagtaaaggttgaagacaaggacttcccctctacctgctcaaagaaaaaagatcgaaaaagcttctgcacaatccacagcacaggctatttgaaaagctggccacccacaaagatggggctggatgaagacaacgaaccagacaatgaggggtgtaacctcagctgcctcgtcgcaattggacgactgcattctcatgtagttccacaaccagtgaacggggaaatcagggtgaaatctatggaatatgtttctcggcacgcgatagatggaaagtttgtttttgtagaccagagggcaacagctattttggcatatttaccacaagaacttctaggcacatcgtgttatgaatattttcaccaagatgacataggacatcttgcagaatgtcataggcaagttttacagacgagagaaaaaattacaactaattgctataaatttaaaatcaaagatggttcttttatcacactacggagtcgatggttcagtttcatgaacccttggaccaaggaagtagaatatattgtctcaactaacactgttgttttagccaacgtcctggaaggcggggacccaaccttcccacagctcacagcatccccccacagcatggacagcatgctgccctctggagaaggtggcccaaagaggacccaccccactgttccagggattccagggggaacccgggctggggcaggaaaaataggccgaatgattgctgaggaaatcatggaaatccacaggataagagggtcatcgccttctagctgtggctccagcccattgaacatcacgagtacgcctccccctgatgcctcttctccaggaggcaagaagattttaaatggagggactccagacattccttccagtggcctactatcaggccaggctcaggagaacccaggttatccatattctgatagttcttctattcttggtgagaacccccacataggtatagacatgattgacaacgaccaaggatcaagtagtcccagtaatgatgaggcagcaatggctgtcatcatgagcctcttggaagcagatgctggactgggtggccctgttgactttagtgacttgccatggccgctgtaa

SEQ ID NO: 2 - DNA, 1911 bp, Human
atggcggcggaagaggaggctgcggcgggaggtaaagtgttgagagaggagaaccagtgcattgctcctgtggtttccagccgcgtgagtccagggacaagaccaacagctatggggtctttcagctcacacatgacagagtttccacgaaaacgcaaaggaagtgattcagacccatcccagtcaggaatcatgacagaaaaagtggtggaaaagctttctcagaatccccttacctatcttctttcaacaaggatagaaatatcagcctccagtggcagcagagtggaagatggtgaacaccaagttaaaatgaaggccttcagagaagctcatagccaaactgaaaagcggaggagagataaaatgaataacctgattgaagaactgtctgcaatgatccctcagtgcaaccccatggcgcgtaaactggacaaacttacagttttaagaatggctgttcaacacttgagatctttaaaaggcttgacaaattcttatgtgggaagtaattatagaccatcatttcttcaggataatgagctcagacatttaatccttaagactgcagaaggcttcttatttgtggttggatgtgaaagaggaaaaattctcttcgtttctaagtcagtctccaaaatacttaattatgatcaggctagtttgactggacaaagcttatttgacttcttacatccaaaagatgttgccaaagtaaaggaacaactttcttcttttgatatttcaccaagagaaaagctaatagatgccaaaactggtttgcaagttcacagtaatctccacgctggaaggacacgtgtgtattctggctcaagacgatcttttttctgtcggataaagagttgtaaaatctctgtcaaagaagagcatggatgcttacccaactcaaagaagaaagagcacagaaaattctatactatccattgcactggttacttgagaagctggcctccaaatattgttggaatggaagaagaaaggaacagtaagaaagacaacagtaattttacctgccttgtggccattggaagattacagccatatattgttccacagaacagtggagagattaatgtgaaaccaactgaatttataacccggtttgcagtgaatggaaaatttgtctatgtagatcaaagggcaacagcgattttaggatatctgcctcaggaacttttgggaacttcttgttatgaatattttcatcaagatgaccacaataatttgactgacaagcacaaagcagttctacagagtaaggagaaaatacttacagattcctacaaattcagagcaaaagatggctcttttgtaactttaaaaagccaatggtttagtttcacaaatccttggacaaaagaactggaatatattgtatctgtcaacactttagttttgggacatagtgagcctggagaagcatcatttttaccttgtagctctcaatcatcagaagaatcctctagacagtcctgtatgagtgtacctggaatgtctactggaacagtacttggtgctggtagtattggaacagatattgcaaatgaaattctggatttacagaggttacagtcttcttcataccttgatgattcgagtccaacaggtttaatgaaagatactcatactgtaaactgcaggagtatgtcaaataaggagttgtttccaccaagtccttctgaaatgggggagctagaggctaccaggcaaaaccagagtactgttgctgtccacagccatgagccactcctcagtgatggtgcacagttggatttcgatgccctatgtgacaatgatgacacagccatggctgcatttatgaattacttagaagcagaggggggcctgggagaccctggggacttcagtgacatccagtggaccctctag

SEQ ID NO: 3 - DNA, 3872 bp, Human
atgagtggccccctagaaggggctgatgggggaggggaccccaggcctggggaatcatttgtcctgggggcgtcccatcccctgggcccccacagcaccggccttgcccaggccccagcctggccgatgacaccgatgccaacagcaatggttcaagtggcaatgagtccaacgggcatgagtctagaggcgcatctcagcggagctcacacagctcctcctcaggcaacggcaaggactcagccctgctggagaccactgagagcagcaagagcacaaactctcagagcccatccccacccagcagttccattgcctacagcctcctgagtgccagctcagagcaggacaacccgtccaccagtggctgcagcagtgaacagtcagcccgggcaaggactcagaaggaactcatgacagcacttcgagagctcaagcttcgactgccgccagagcgccggggcaagggccgctctgggaccctggccacgctgcagtacgcactggcctgtgtcaagcaggtgcaggccaaccaggaatactaccagcagtggagcctggaggagggcgagccttgctccatggacatgtccacctataccctggaggagctggagcacatcacgtctgagtacacacttcagaaccaggataccttctcagtggctgtctccttcctgacgggccgaatcgtctacatttcggagcaggcagccgtcctgctgcgttgcaagcgggacgtgttccggggtacccgcttctctgagctcctggctccccaggatgtgggagtcttctatggttccactgctccatctcgcctgcccacctggggcacaggggcctcagcaggttcaggcctcagggactttacccaggagaagtccgtcttctgccgtatcagaggaggtcctgaccgggatccagggcctcggtaccagccattccgcctaaccccgtatgtgaccaagatccgggtctcagatggggcccctgcacagccgtgctgcctgctgattgcagagcgcatccattcgggttacgaagctccccggataccccctgacaagaggattttcactacgcggcacacacccagctgcctcttccaggatgtggatgaaagggctgcccccctgctgggctacctgccccaggacctcctgggggccccagtgctcctgttcctgcatcctgaggaccgacccctcatgctggctatccacaagaagattctgcagttggcgggccagccctttgaccactcccctatccgcttctgtgcccgcaacggggagtatgtcaccatggacaccagctgggctggctttgtgcacccctggagccgcaaggtagccttcgtgttgggccgccacaaagtacgcacggcccccctgaatgaggacgtgttcactcccccggcccccagcccagctccctccctggacactgatatccaggagctgtcagagcagatccaccggctgctgctgcagcccgtccacagccccagccccacgggactctgtggagtcggcgccgtgacatccccaggccctctccacagccctgggtcctccagtgatagcaacgggggtgatgcagaggggcctgggcctcctgcgccagtgactttccagcagatctgtaaggatgtgcatctggtgaagcaccagggccagcagctttttattgagtctcgggcccggcctcagtcccggccccgcctccctgctacaggcacgttcaaggccaaggcccttccctgccaatccccagacccagagctggaggcgggttctgctcccgtccaggccccactagccttggtccctgaggaggccgagaggaaagaagcctccagctgctcctaccagcagatcaactgcctggacagcatcctcaggtacctggagagctgcaacctccccagcaccactaagcgtaaatgtgcctcctcctcctcctataccacctcctcagcctctgacgacgacaggcagaggacaggtccagtctctgtggggaccaagaaagatccgccgtcagcagcgctgtctggggagggggccaccccacggaaggagccagtggtgggaggcaccctgagcccgctcgccctggccaataaggcggagagtgtggtgtccgtcaccagtcagtgtagcttcagctccaccatcgtccatgtgggagacaagaagcccccggagtcggacatcatcatgatggaggacctgcctggcctagccccaggcccagcccccagcccagcccccagccccacagtagcccctgacccagccccagacgcctaccgtccagtggggctgaccaaggccgtgctgtccctgcacacacagaaggaagagcaagccttcctcagccgcttccgagacctgggcaggctgcgtggactcgacagctcttccacagctccctcagcccttggcgagcgaggctgccaccacggccccgcacccccaagccgccgacaccactgccgatccaaagccaagcgctcacgccaccaccagaaccctcgggctgaagcgccctgctatgtctcacacccctcacccgtgccaccctccaccccctggcccaccccaccagccactacccccttcccagcggttgtccagccctaccctctcccagtgttctctcctcgaggaggcccccagcctcttccccctgctcccacatctgtgcccccagctgctttccccgcccctttggtgaccccaatggtggccttggtgctccctaactatctgttcccaaccccatccagctatccttatggggcactccagacccctgctgaagggcctcccactcctgcctcgcactccccttctccatccttgcccgccctcgccccgagtcctcctcaccgcccggactctccactgttcaactcgagatgcagctctccactccagctcaatctgctgcagctggaggagctcccccgtgctgagggggctgctgttgcaggaggccctgggagcagtgccgggcccccacctcccagtgcggaggctgctgagccagaggccagactggcggaggtcactgagtcctccaatcaggacgcactttccggctccagtgacctgctcgaacttctgctgcaagaggactcgcgctccggcacaggctccgcagcctcgggctccttgggctctggcttgggctctgggtctggttcaggctcccatgaagggggcagcacctcagccagcatcactcgcagcagccagagcagccacacaagcaaatactttggcagcatcgactcttccgaggctgaggctggggctgctcggggcggggctgagcctggggaccaggtgattaagtacgtgctccaggatcccatttggctgctcatggccaatgctgaccagcgcgtcatgatgacctaccaggtgccctccagggacatgacctctgtgctgaagcaggatcgggagcggctccgagccatgcagaagcagcagcctcggttttctgaggaccagcggcgggaactgggtgctgtgcactcctgggtccggaagggccaactgcctcgggctcttgatgtgatggcctgtgtggactgtgggagcagcacccaagatcctggtcaccctgatgacccactcttctcagagctggatggactggggctggagcccatggaagagggtggaggcgagcagggcagcagcggtggcggcagtggtgagggagagggctgcgaggaggcccaaggcggggccaaggcttcaagctctcaggacttggctatggaggaggaggaagaaggcaggagctcatccagtccagccttacctacagcaggaaactgcaccagctag

SEQ ID NO: 4 - DNA, 3768 bp, Human
atgaatggatacgcggaatttccgcccagccccagtaaccccaccaaggagcccgtggagccccagcccagccaggtcccactgcaggaagatgtggacatgagcagtggctccagtggacatgagaccaacgaaaactgctccacggggcgggactcgcagggcagtgactgtgacgacagtgggaaggagctggggatgctggtggagccaccggatgcccgccagagtccagatacctttagcctgatgatggcaaaatctgaacacaacccatctacaagtggctgcagtagcgaccagtcttcgaaagtggacacacacaaagaactgataaaaacactaaaggagctgaaggtccacctccctgcagacaagaaggccaagggcaaggccagtacgctggccaccttgaagtacgccctcaggagcgtgaagcaggtgaaagccaatgaagagtattaccagctgctgatgtccagcgagggtcacccctgtggagcagacgtgccctcctacaccgtggaggagatggagagcgttacctctgagcacattgtgaagaatgccgatatgtttgcggtggccgtgtccctggtgtctgggaagatcctgtacatctctgaccaggttgcatccatatttcactgtaaaagagatgccttcagcgatgccaagtttgtggagttcctggcgcctcacgatgtgggcgtgttccacagtttcacctccccgtacaagcttcccttgtggagcatgtgcagtggagcagattcttttactcaagaatgcatggaggagaaatctttcttttgccgtgtcagtgtccggaaaagccacgagaatgaaatccgctaccaccccttccgcatgacgccctacctggtcaaggtgcgggaccaacaaggtgctgagagtcagctttgctgccttctgctggcagagagagtgcactctggttatgaagcccctagaattcctcctgaaaagagaatttttacaaccacccatacaccaaattgtttgttccaggatgtggatgaaagggcggtccctctcctgggctacctacctcaggacctgattgaaaccccagtgctcgtgcagctccaccctagtgacaggcccttgatgctggccatccacaaaaagatcctgcagtcaggcgggcagcctttcgactattctcccattcggtttcgcgcccggaacggagagtacatcacgttggacaccagctggtccagcttcatcaacccatggagcaggaaaatctccttcatcattgggaggcacaaagtcagggtgggccctttgaatgaggacgtgtttgcagcccacccctgcacagaggagaaggccctgcaccccagcattcaggagctcacagagcagatccaccggctcctgctgcagcccgtcccccacagcggctccagtggctacgggagtctgggcagcaacgggtcccacgagcaccttatgagccagacctcctccagcgacagcaacggccatgaggactcacgccggaggagagccgaaatttgtaaaaatggtaacaagaccaaaaatagaagtcattattctcatgaatctggagaacaaaagaaaaaatccgttacagaaatgcaaactaatcccccagctgagaagaaagctgtccctgccatggaaaaggacagcctgggggtcagcttccccgaggagttggcctgcaagaaccagcccacctgctcctaccagcagatcagctgcttggacagcgtcatcaggtacttggagagctgcaatgaggctgccaccctgaagaggaaatgcgagttcccagcaaacgtcccagcgctaaggtccagtgataagcggaaggccacagtcagcccagggccacacgctggagaggcagagccgccctccagggtgaacagccgcacgggagtaggtacgcacctgacctcgctggcactgccgggcaaggcagagagtgtggcgtcgctcaccagccagtgcagctacagcagcaccatcgtccatgtgggagacaagaagccgcagccggagttagagatggtggaagatgctgcgagtgggccagaatccctggactgcctggcgggccctgccctggcctgtggtctcagccaagagaaggagcccttcaagaagctgggcctcaccaaggaggtactcgctgcacacacacagaaggaggagcagagcttcctgcagaagttcaaagaaataagaaaactcagcattttccagtcccactgccattactacttgcaagaaagatccaaggggcagccaagtgaacgaactgcccctggactaagaaatacttccggaatagattcaccttggaaaaaaacaggaaagaacagaaaattgaagtccaagcgggtcaaacctcgagactcatctgagagcaccggatctggggggcccgtgtccgcccggcccccgctggtgggcttgaacgccacagcctggtcaccctcagacacgtcccagtccagctgcccagccgtgccctttcccgccccagtgccagcagcttattcactgcccgtgtttccagcgccagggactgtggcagcacccccggcacctccccacgccagcttcacagtgcctgctgtgcccgtggacctccagcaccagtttgcagtccagcccccacctttccctgcccctttggcgcctgtcatggcattcatgctacccagttattccttcccctcggggaccccaaacctgccccaggccttcttccccagccagcctcagtttccgagccaccccacactcacatccgagatggcctctgcctcacagcctgagttccccagccggacctcgatccccagacagccatgtgcttgtccagccacccgggccaccccaccatcggccatgggtagggcctccccaccgctctttcagtcccgcagcagctcgcccctgcagctcaacctgctgcagctggaggaagcccctgagggtggcactggagccatggggaccacaggggccacagagacagcagctgtaggggcggactgcaaacctggcacttctcgggaccagcagccgaaggcgcctctgacccgtgatgaaccctcagacacacagaacagtgacgccctttccacgtcaagcggcctcctaaacctcctgctgaatgaggacctctgctcagcctcgggctctgctgcttcggagtctctgggctccggctcactgggctgcgacgcctccccgagtggggcaggcagtagtgacacaagtcataccagcaaatattttggaagcattgactcctcagagaataatcacaaagcaaaaatgaacactggtatggaagaaagtgagcatttcattaagtgcgtcctgcaggatcccatctggctgctgatggcagatgcggacagcagcgtcatgatgacgtaccagctgccttcccgaaatttagaagcggttttgaaggaggacagagagaagctgaagctcctacagaaactccagcccaggttcacggagagtcagaagcaggagctgcgcgaggtccaccagtggatgcagacgggcggcctgcccgcagccatcgacgtggcagaatgtgtttactgtgaaaacaaggaaaaaggtaatatttgcataccatatgaggaagatattccttctctgggactcagcgaagtgtcggacaccaaagaagacgaaaatggatcccccttgaatcacaggatcgaagagcagacgtaa

SEQ ID NO: 5 - DNA, 3633 bp, Human
atgccccgcggggaagctcctggccccgggagacggggggctaaggacgaggccctgggcgaagaatcgggggagcggtggagccccgagttccatctgcagaggaaattggcggacagcagccacagtgaacagcaagatcgaaacagagtttctgaagaacttatcatggttgtccaagaaatgaaaaaatacttcccctcggagagacgcaataaaccaagcactctagatgccctcaactatgctctccgctgtgtccacagcgttcaagcaaacagtgagtttttccagattctcagtcagaatggagcacctcaggcagatgtgagcatgtacagtcttgaggagctggccactatcgcttcagaacacacttccaaaaacacagatacctttgtggcagtattttcatttctgtctggaaggttagtgcacatttctgaacaggctgctttgatcctgaatcgtaagaaagatgtcctggcgtcttctcactttgttgacctgcttgcacctcaagacatgagggtattctacgcgcacactgccagagctcagcttcctttctggaacaactggacccaaagagcagctgcacggtatgaatgtgctccggtgaaaccttttttctgcaggatccgtggaggtgaagacagaaagcaagagaagtgtcactccccattccggatcatcccctatctgattcatgtacatcaccctgcccagccagaattggaatcggaaccttgctgtctcactgtggttgaaaagattcactctggttatgaagctcctcggatcccagtgaataaaagaatcttcaccaccacacacaccccagggtgtgtttttcttgaagtagatgaaaaagcagtgcctttgctgggttacctacctcaggacctgattggaacatcgatcctaagctacctgcaccctgaagatcgttctctgatggttgccatacaccaaaaagttttgaagtatgcagggcatcctccctttgaacattctcccattcgattttgtactcaaaacggagactacatcatactggattccagttggtccagctttgtgaatccctggagccggaagatttctttcatcattggtcggcataaagttcgaacgagcccactaaatgaggatgtttttgctaccaaaattaaaaagatgaacgataatgacaaagacataacagaattacaagaacaaatttacaaacttctcttacagccagttcacgtgagcgtgtccagcggctacgggagcctggggagcagcgggtcgcaggagcagcttgtcagcatcgcctcctccagtgaggccagtgggcaccgtgtggaggagacgaaggcggagcagatgaccttgcagcaggtctatgccagtgtgaacaaaattaaaaatctgggtcagcagctctacattgagtcaatgaccaaatcatcattcaagccagtgacggggacacgcacagaaccgaatggtggtggtgagtcagcgaatggtggtggtgaatgtaagacctttacttccttccaccaaacactgaaaaacaatagtgtgtacactgagccctgtgaggatttgaggaacgatgagcacagcccatcctatcaacagatcaactgtatcgacagtgtcatcagatacctgaagagctacaacattccagctttgaaaagaaagtgtatctcctgtacaaatacaacttcttcctcctcagaagaagacaaacagaaccacaaggcagatgatgtccaagccttacaagctggtttgcaaatcccagccatacctaaatcagaaatgccaacaaatggacggtccatagacacaggaggaggagctccacagatcctgtccacggcgatgctgagcttggggtcgggcataagccaatgcggttacagcagcaccattgtccatgtcccacccccagagacagccagggatgctaccctcttctgtgagccctggaccctgaacatgcagccagcccctttgacctcggaagaatttaaacacgtggggctcacagcggctgttctgtcagcgcacacccagaaggaagagcagaattatgttgataaattccgagaaaagatcctgtcatcaccctacagctcctatcttcagcaagaaagcaggagcaaagctaaatattcatattttcaaggagattctacttccaagcagacgcggtcggccggctgcaggaaagggaagcacaagcggaagaagctgccggagccgccagacagcagcagctcgaacaccggctctggtccccgcaggggagcgcatcagaacgcacagccctgctgcccctccgcggcctcctctccgcacacctcgagcccgaccttcccacctgccgccatggtgcccagccaggccccttacctcgtcccagcttttcccctcccagccgcgacctcacccggaagagaatacgcagcccccggaactgcaccggaaggcctgcatgggctgcccttgtccgagggcttgcagccttacccagctttcccttttccttacttggatacttttatgaccgttttcctgcctgacccccctgtctgtcctctgttgtcgccatcgtttttgccatgtccattcctgggggcgacagcctcttctgcgatatcaccctcaatgtcgtcagcaatgagtccaactctggacccacccccttcagtcaccagccaaaggagagaggaggaaaagtgggaggcacaaagcgaggggcacccgttcattacttcgagaagcagctcacccttgcagttaaacttacttcaggaagagatgcccagaccctctgaatctccagatcagatgagaaggaacacgtgcccacaaactgagtatcagtgtgttacaggcaacaatggcagtgagagcagtcctgctactaccggtgcactgtccacggggtcacctcccagggagaatccatcccatcctactgccagcgctctgtccacaggatcgcctcccatgaagaatccatcccatcctactgccagcgctctgtccacaggatcgcctcccatgaagaatccatcccatcctactgccagcacactgtccatgggattgcctcccagcaggactccatcccatcctactgccactgttctgtccacggggtcacctcccagcgaatccccatccagaactggttcagcagcatcaggaagcagcgacagcagtatataccttactagtagtgtttattcttctaaaatctcccaaaatgggcagcaatctcaggacgtacagaaaaaagaaacatttcctaatgtcgccgaagagcccatctggagaatgatacggcagacacctgagcgcattctcatgacataccaggtacctgagagggttaaagaagttgtactaaaagaagacctggaaaagctagaaagtatgaggcagcagcagccccagttttctcatgggcaaaaggaggagctggctaaggtgtataattggattcaaagccagactgtcactcaagaaatcgacattcaagcctgtgtcacttgtgaaaatgaagattcagctgatggtgcggccacatcctgtggtcaggttctggtagaagacagctgttga

SEQ ID NO: 6 - DNA, 2541 bp, Human
atgttgtttaccgtaagctgtagtaaaatgagctcgattgttgacagagatgacagtagtatttttgatgggttggtggaagaagatgacaaggacaaagcgaaaagagtatctagaaacaaatctgaaaagaaacgtagagatcaatttaatgttctcattaaagaactgggatccatgcttcctggtaatgctagaaagatggacaaatctactgttctgcagaaaagcattgattttttacgaaaacataaagaaatcactgcacagtcagatgctagtgaaattcgacaggactggaaacctacattccttagtaatgaagagtttacacaattaatgttagaggctcttgatggtttttttttagcaatcatgacagatggaagcataatatatgtgtctgagagtgtaacttcattacttgaacatttaccatctgatcttgtggatcaaagtatatttaattttatcccagaaggggaacattcagaggtttataaaatactctctactcatctgctggaaagtgattcattaaccccagaatatttaaaatcaaaaaatcagttagaattctgttgtcacatgctgcgaggaacaatagacccaaaggagccatctacctatgaatatgtaaaatttataggaaatttcaaatctttaaacagtgtatcctcttcagcacacaatggttttgaaggaactatacaacgcacacataggccatcttatgaagatagagtttgttttgtagctactgtcaggttagctacacctcagttcatcaaggaaatgtgcactgttgaagaacccaatgaagagtttacatctagacatagtttagaatggaagtttctgtttctagatcacagggcaccacccataatagggtatttgccatttgaagttctgggaacatcaggctatgattactatcatgtggatgacctagaaaatttggcaaaatgtcatgagcacttaatgcaatatgggaaaggcaaatcatgttattataggttcctgactaaggggcaacagtggatttggcttcagactcattattatatcacttaccatcagtggaattcaaggccagagtttattgtttgtactcacactgtagtaagttatgcagaagttagggctgaaagacgacgagaacttggcattgaagagtctcttcctgagacagctgctgacaaaagccaagattctgggtcagataatcgtataaacacagtcagtctcaaggaagcattggaaaggtttgatcacagcccaaccccttctgcctcttctcggagttcaagaaaatcatctcacacggccgtctcagacccttcctcaacaccaaccaagatcccgacggatacgagcactccacccaggcagcatttaccagctcatgagaagatggtgcaaagaaggtcatcatttagtagtcagtccataaattcccagtctgttggttcatcattaacacagccagtgatgtctcaagctacaaatttaccaattccacaaggcatgtcccagtttcagttttcagctcaattaggagccatgcaacatctgaaagaccaattggaacaacggacacgcatgatagaagcaaatattcatcggcaacaagaagaactaagaaaaattcaagaacaacttcagatggtccatggtcaggggctgcagatgtttttgcaacaatcaaatcctgggttgaattttggttccgttcaactttcttctggaaattcatctaatatccagcaacttgcacctataaatatgcaaggccaagttgttcctactaaccagattcaaagtggaatgaatactggacacattggcacaactcagcacatgatacaacaacagactttacagagtacatcaactcagagtcaacaaaatgtactgagtgggcacagtcagcaaacatctctacccagtcagacacagagcactcttacagccccactgtataacactatggtgatttctcagcctgcagccggaagcatggtccagattccatctagtatgccacaaaacagcacccagagtgctgcagtaactacattcactcaggacaggcagataagattttctcaaggtcaacaacttgtgaccaaattagtgactgctcctgtagcttgtggggcagtcatggtacctagtactatgcttatgggccaggtggtgactgcatatcctacttttgctacacaacagcaacagtcacagacattgtcagtaacgcagcagcagcagcagcagagctcccaggagcagcagctcacttcagttcagcaaccatctcaggctcagctgacccagccaccgcaacaatttttacagacttctaggttgctccatgggaatccctcaactcaactcattctctctgctgcatttcctctacaacagagcaccttccctcagtcacatcaccagcaacatcagtctcagcaacagcagcaactcagccggcacaggactgacagcttgcccgacccttccaaggttcaaccacagtag

SEQ ID NO: 7 - DNA, 2475 bp, Human
atggatgaagatgagaaagacagagccaagagagcttctcgaaacaagtctgagaagaagcgtcgggaccagttcaatgttctcatcaaagagctcagttccatgctccctggcaacacgcggaaaatggacaaaaccaccgtgttggaaaaggtcatcggatttttgcagaaacacaatgaagtctcagcgcaaacggaaatctgtgacattcagcaagactggaagccttcattcctcagtaatgaagaattcacccagctgatgttggaggcattagatggcttcattatcgcagtgacaacagacggcagcatcatctatgtctctgacagtatcacgcctctccttgggcatttaccgtcggatgtcatggatcagaatttgttaaatttcctcccagaacaagaacattcagaagtttataaaatcctttcttcccatatgcttgtgacggattccccctccccagaatacttaaaatctgacagcgatttagagttttattgccatcttctcagaggcagcttgaacccaaaggaatttccaacttatgaatacataaaatttgtaggaaattttcgctcttacaacaatgtgcctagcccctcctgtaatggttttgacaacaccctttcaagaccttgccgggtgccactaggaaaggaggtttgcttcattgccaccgttcgtctggcaacaccacaattcttaaaggaaatgtgcatagttgacgaacctttagaggaattcacttcaaggcatagcttggaatggaaatttttatttctggatcacagagcacctccaatcataggatacctgccttttgaagtgctgggaacctcaggctatgactactaccacattgatgacctggagctcctggccaggtgtcaccagcacctgatgcagtttggcaaagggaagtcgtgttgctaccggtttctgaccaaaggtcagcagtggatctggctgcagactcactactacatcacctaccatcagtggaactccaagcccgagttcatcgtgtgcacacactcggtggtcagttacgcagatgtccgggtggaaaggaggcaggagctggctctggaagacccgccatccgaggccctccactcctcagcactaaaggacaagggctcaagcctggaacctcggcagcactttaacacactcgacgtgggtgcctcgggccttaataccagtcattcgccatcggcgtcctcaagaagttcccacaaatcctcgcacacagccatgtcagaacccacctccactcccaccaagctgatggcagaggccagcaccccggctttgccaagatcagccaccctgccccaagagttacctgtccccgggctcagccaggcagccaccatgccggcccctctgccttccccatcgtcctgcgacctcacacagcagctcctgcctcagaccgttctgcagagcacgcccgctcccatggcacagttttcggcacagttcagcatgttccagaccatcaaagaccagctagagcagcggacgcggatcctgcaggccaatatccggtggcaacaggaagagctccacaagatccaggagcagctctgcctggtccaggactccaacgtccagatgttcctgcagcagccagctgtatccctgagcttcagcagcacccagcgacctgaggctcagcagcagctacagcaaaggtcagctgcagtgactcagccccagctcggggcgggcccccaacttccagggcagatctcctctgcccaggtcacaagccagcacctgctcagagaatcaagtgtgatatcaacccagggtccaaagccaatgagaagctcacagctaatgcagagcagcggccgctctggaagcagcctagtgtccccgttcagcagcgccacagctgcgctcccgccaagtctgaatctgaccacacctgcttccacctcccaggatgccagccagtgccagcccagcccagacttcagccatgatcggcagctcaggctgttgctgagccagcccatccagcccatgatgcccgggtcctgtgacgcaaggcagccctcggaagtcagcaggacgggacggcaagtcaagtacgcccagagccagaccgtgtttcaaaatccagacgcacaccccgccaacagcagcagcgccccgatgcccgtcctgctgatggggcaggcggtgctccaccccagcttccctgcctcccaaccatcgcccctgcagcctgcacaggcccggcagcagccaccgcagcactacctgcaggtacaggcaccaacctctttgcacagtgagcagcaggactcgctacttctctccacctactcacaacagccagggaccctgggctacccccaaccacccccagcacagccccagcccctacgtcctccccgaagggtcagcagtctgtctgagtcgtcaggcctccagcagccgccccgataa

SEQ ID NO: 8 - DNA, 1761 bp, Human
atgggggtgaacgccgtgcactggttccgaaaggggctccggctccacgacaaccccgccctgaaggagtgcattcagggcgccgacaccatccgctgcgtctacatcctggacccctggttcgccggctcctccaatgtgggcatcaacaggtggcgatttttgcttcagtgtcttgaggatcttgatgccaatctacgaaaattaaactcccgtctgtttgtgattcgtggacaaccagcagatgtgtttcccaggcttttcaaggaatggaacattactaaactttcaattgagtatgattctgagccctttggaaaggaacgagacgcagctattaagaaactggcaactgaagctggagtagaagtcattgtaagaatttcacatacattatatgacctagacaagatcatagaactcaatggtggacaaccgcctctaacttataaaagattccagactctcatcagcaaaatggaaccactagagataccagtagagacaattacttcagaagtgatagaaaagtgcacaactcctctgtctgatgaccatgatgagaaatatggagtcccttcactggaagagctaggttttgatacagatggcttatcctctgcagtgtggccaggtggagaaactgaagcacttactcgtttggaaaggcatttggaaagaaaagcttgggtggcaaattttgaaagacctcgaatgaatgcgaattctctgcttgcaagccctactggacttagtccttatctccgatttggttgtttgtcatgtcgactgttttacttcaaactaacagatctctacaaaaaggtaaagaagaacagttcccctcccctttccctttatgggcaactgttatggcgtgaatttttctatacagcagcaacaaataatccacgctttgataaaatggaaggaaaccctatctgtgttcagattccttgggataaaaatcctgaggctttagccaaatgggcggaaggccggacaggctttccatggattgatgccatcatgacacagcttcgtcaggagggttggattcatcatctagccaggcatgcagttgcttgcttcctgacacgaggggacctgtggattagttgggaagaaggaatgaaggtatttgaagaattattgcttgatgcagattggagcataaatgctggaagttggatgtggctgtcttgtagttccttttttcaacagttttttcactgctattgccctgttggttttggtaggagaacagatcccaatggagactatatcaggcgttatttgcctgtcctaagaggcttccctgcaaaatatatctatgatccctggaatgcaccagaaggtatccaaaaggtagccaaatgtttgataggagttaattatcctaaaccaatggtgaaccatgctgaggcaagccgtttgaatatcgaaaggatgaaacagatctatcagcagctttcacgatatagaggactaggtcttctggcatcagtaccttctaatcctaatgggaatggaggcttcatgggatattctgcagaaaatatcccaggttgtagcagcagtggaagttgctctcaagggagtggtattttacactatgctcatggcgacagtcagcaaactcacctgttgaagcaaggaagaagctccatgggcactggtctcagtggtgggaaacgtcctagtcaggaagaggacacacagagtattggtcctaaagtccagagacagagcactaattag

SEQ ID NO: 9 - DNA, 1845 bp, Human
atgggcggggtccacgtcgcctaccggggcggagcgggggtggctggagcagtctggacagtcatggcggcgactgtggcgacggcggcagctgtggccccggcgccagcgcccggcacggacagcgcctcttcggtgcactggttccgcaaagggctgcgactccacgacaacccggcgttgctggcggccgtgcgcggggcgcgctgcgtgcgctgcgtttacattctcgacccgtggttcgcggcctcctcctcagtcgggatcaaccgatggaggttcctacttcagtctctggaagatttggacacaagtttaaggaaactgaactcccgcctgtttgtagtccggggacagccagccgacgtgttcccaaggctgttcaaggaatggggagtgacccgcttgacctttgaatatgactctgaaccctttgggaaagaacgggatgcagccatcatgaagatggccaaggaggctggtgtggaagtagtgacggagaattctcataccctctatgacctggacaggatcattgagctgaatgggcagaagccaccccttacatacaagcgctttcaggccatcatcagccgcatggagctgcccaagaagccagtgggcttggtgaccagccagcagatggagagctgcagggccgagatccaggagaaccacgacgagacctacggcgtgccctccctggaggagctggggttccccactgaaggacttggtccagctgtctggcagggaggagagacagaagctctggcccgcctggataagcacttggaacggaaggcctgggttgccaactatgagagaccccgaatgaacgccaactccctcctggccagccccacaggcctcagcccctacctgcgctttggttgtctctcctgccgcctcttctactaccgcctgtgggacctgtataaaaaggtgaagcggaacagcacacctcccctctccctatttgggcaactcctatggcgagagttcttctacacggcagctaccaacaaccccaggtttgaccgcatggaggggaaccccatctgcatccagatcccctgggaccgcaatcctgaggccctggccaagtgggctgagggcaagacaggcttcccttggattgatgccatcatgacccaactgaggcaggagggctggatccaccacctggcccggcatgccgtggcctgcttcctgacccgcggggacctctgggtcagctgggagagcggggtccgggtatttgatgagctgctcctggatgcagatttcagcgtgaacgcaggcagctggatgtggctgtcctgcagtgctttcttccagcagttcttccactgctactgccctgtgggctttggccgtcgcacggaccccagtggggactacatcaggcgatacctgcccaaattgaaagcgttcccctctcgatacatctatgagccctggaatgccccagagtcaattcagaaggcagccaagtgcatcattggtgtggactacccacggcccatcgtcaaccatgccgagaccagccggcttaacattgaacgaatgaagcagatttaccagcagctttcgcgctaccggggactctgtctactggcatctgtcccttcctgtgtggaagacctcagtcaccctgtggcagagcccagctcgagccaggctggcagcatgagcagtgcaggcccaagaccactacccagtggcccagcatcccccaaacgcaagctggaagcagccgaggaaccacctggtgaagaactcagcaaacgggcccgggtggcagagttgccaaccccagagctgccgagcaaggatgcctga

SEQ ID NO: 10 - DNA, 1845 bp, Human
atgacgaccctggactccaacaacaacacaggtggcgtcatcacctacattggctccagtggctcctccccaagccgcaccagccctgaatccctctatagtgacaactccaatggcagcttccagtccctgacccaaggctgtcccacctacttcccaccatcccccactggctccctcacccaagacccggctcgctcctttgggagcattccacccagcctgagtgatgacggctccccttcttcctcatcttcctcgtcgtcatcctcctcctccttctataatgggagcccccctgggagtctacaagtggccatggaggacagcagccgagtgtcccccagcaagagcaccagcaacatcaccaagctgaatggcatggtgttactgtgtaaagtgtgtggggacgttgcctcgggcttccactacggtgtgcacgcctgcgagggctgcaagggctttttccgtcggagcatccagcagaacatccagtacaaaaggtgtctgaagaatgagaattgctccatcgtccgcatcaatcgcaaccgctgccagcaatgtcgcttcaagaagtgtctctctgtgggcatgtctcgagacgctgtgcgttttgggcgcatccccaaacgagagaagcagcggatgcttgctgagatgcagagtgccatgaacctggccaacaaccagttgagcagccagtgcccgctggagacttcacccacccagcaccccaccccaggccccatgggcccctcgccaccccctgctccggtcccctcacccctggtgggcttctcccagtttccacaacagctgacgcctcccagatccccaagccctgagcccacagtggaggatgtgatatcccaggtggcccgggcccatcgagagatcttcacctacgcccatgacaagctgggcagctcacctggcaacttcaatgccaaccatgcatcaggtagccctccagccaccaccccacatcgctgggaaaatcagggctgcccacctgcccccaatgacaacaacaccttggctgcccagcgtcataacgaggccctaaatggtctgcgccaggctccctcctcctaccctcccacctggcctcctggccctgcacaccacagctgccaccagtccaacagcaacgggcaccgtctatgccccacccacgtgtatgcagccccagaaggcaaggcacctgccaacagtccccggcagggcaactcaaagaatgttctgctggcatgtcctatgaacatgtacccgcatggacgcagtgggcgaacggtgcaggagatctgggaggatttctccatgagcttcacgcccgctgtgcgggaggtggtagagtttgccaaacacatcccgggcttccgtgacctttctcagcatgaccaagtcaccctgcttaaggctggcacctttgaggtgctgatggtgcgctttgcttcgttgttcaacgtgaaggaccagacagtgatgttcctaagccgcaccacctacagcctgcaggagcttggtgccatgggcatgggagacctgctcagtgccatgttcgacttcagcgagaagctcaactccctggcgcttaccgaggaggagctgggcctcttcaccgcggtggtgcttgtctctgcagaccgctcgggcatggagaattccgcttcggtggagcagctccaggagacgctgctgcgggctcttcgggctctggtgctgaagaaccggcccttggagacttcccgcttcaccaagctgctgctcaagctgccggacctgcggaccctgaacaacatgcattccgagaagctgctgtccttccgggtggacgcccagtga

SEQ ID NO: 11 - DNA, 1740 bp, Human
atggaggtgaatgcaggaggtgtgattgcctatatcagttcttccagctcagcctcaagccctgcctcttgtcacagtgagggttctgagaatagtttccagtcctcctcctcttctgttccatcttctccaaatagctctaattctgataccaatggtaatcccaagaatggtgatctcgccaatattgaaggcatcttgaagaatgatcgaatagattgttctatgaaaacaagcaaatcgagtgcacctgggatgacaaaaagtcatagtggtgtgacaaaatttagtggcatggttctactgtgtaaagtctgtggggatgtggcgtcaggattccactatggagttcatgcttgcgaaggctgtaagggtttctttcggagaagtattcaacaaaacatccagtacaagaagtgcctgaagaatgaaaactgttctataatgagaatgaataggaacagatgtcagcaatgtcgcttcaaaaagtgtctgtctgttggaatgtcaagagatgctgttcggtttggtcgtattcctaagcgtgaaaaacagaggatgctaattgaaatgcaaagtgcaatgaagaccatgatgaacagccagttcagtggtcacttgcaaaatgacacattagtagaacatcatgaacagacagccttgccagcccaggaacagctgcgacccaagccccaactggagcaagaaaacatcaaaagctcttctcctccatcttctgattttgcaaaggaagaagtgattggcatggtgaccagagctcacaaggatacctttatgtataatcaagagcagcaagaaaactcagctgagagcatgcagccccagagaggagaacggattcccaagaacatggagcaatataatttaaatcatgatcattgcggcaatgggcttagcagccattttccctgtagtgagagccagcagcatctcaatggacagttcaaagggaggaatataatgcattacccaaatggtcatgccatttgtattgcaaatggacattgtatgaacttctccaatgcttatactcaaagagtatgtgatagagttccgatagatggattttctcagaatgagaacaagaatagttacctgtgcaacactggaggaagaatgcatctggtttgtccaatgagtaagtctccatatgtggatcctcataaatcaggacatgaaatctgggaagaattttcgatgagcttcactccagcagtgaaagaagtggtggaatttgcaaagcgtattcctgggttcagagatctctctcagcatgaccaggtcaaccttttaaaggctgggacttttgaggttttaatggtacggttcgcatcattatttgatgcaaaggaacgtactgtcacctttttaagtggaaagaaatatagtgtggatgatttacactcaatgggagcaggggatctgctaaactctatgtttgaatttagtgagaagctaaatgccctccaacttagtgatgaagagatgagtttgtttacagctgttgtcctggtatctgcagatcgatctggaatagaaaacgtcaactctgtggaggctttgcaggaaactctcattcgtgcactaaggaccttaataatgaaaaaccatccaaatgaggcctctatttttacaaaactgcttctaaagttgccagatcttcgatctttaaacaacatgcactctgaggagctcttggcctttaaagttcacccttaa

SEQ ID NO: 12 - DNA, 1572 bp, Human
atggagtcagctccggcagcccccgaccccgccgccagcgagccaggcagcagcggcgcggacgcggccgccggctccagggagaccccgctgaaccaggaatccgcccgcaagagcgagccgcctgccccggtgcgcagacagagctattccagcaccagcagaggtatctcagtaacgaagaagacacatacatctcaaattgaaattattccatgcaagatctgtggagacaaatcatcaggaatccattatggtgtcattacatgtgaaggctgcaagggctttttcaggagaagtcagcaaagcaatgccacctactcctgtcctcgtcagaagaactgtttgattgatcgaaccagtagaaaccgctgccaacactgtcgattacagaaatgccttgccgtagggatgtctcgagatgctgtaaaatttggccgaatgtcaaaaaagcagagagacagcttgtatgcagaagtacagaaacaccggatgcagcagcagcagcgcgaccaccagcagcagcctggagaggctgagccgctgacgcccacctacaacatctcggccaacgggctgacggaacttcacgacgacctcagtaactacattgacgggcacacccctgaggggagtaaggcagactccgccgtcagcagcttctacctggacatacagccttccccagaccagtcaggtcttgatatcaatggaatcaaaccagaaccaatatgtgactacacaccagcatcaggcttctttccctactgttcgttcaccaacggcgagacttccccaactgtgtccatggcagaattagaacaccttgcacagaatatatctaaatcgcatctggaaacctgccaatacttgagagaagagctccagcagataacgtggcagacctttttacaggaagaaattgagaactatcaaaacaagcagcgggaggtgatgtggcaattgtgtgccatcaaaattacagaagctatacagtatgtggtggagtttgccaaacgcattgatggatttatggaactgtgtcaaaatgatcaaattgtgcttctaaaagcaggttctctagaggtggtgtttatcagaatgtgccgtgcctttgactctcagaacaacaccgtgtactttgatgggaagtatgccagccccgacgtcttcaaatccttaggttgtgaagactttattagctttgtgtttgaatttggaaagagtttatgttctatgcacctgactgaagatgaaattgcattattttctgcatttgtactgatgtcagcagatcgctcatggctgcaagaaaaggtaaaaattgaaaaactgcaacagaaaattcagctagctcttcaacacgtcctacagaagaatcaccgagaagatggaatactaacaaagttaatatgcaaggtgtctaccttaagagccttatgtggacgacatacagaaaagctaatggcatttaaagcaatatacccagacattgtgcgacttcattttcctccattatacaaggagttgttcacttcagaatttgagccagcaatgcaaattgatgggtaa

SEQ ID NO: 13 - DNA, 1380 bp, Human
atgcgagcacaaattgaagtgataccatgcaaaatttgtggcgataagtcctctgggatccactacggagtcatcacatgtgaaggctgcaagggattctttaggaggagccagcagaacaatgcttcttattcctgcccaaggcagagaaactgtttaattgacagaacgaacagaaaccgttgccaacactgccgactgcagaagtgtcttgccctaggaatgtcaagagatgctgtgaagtttgggaggatgtccaagaagcaaagggacagcctgtatgctgaggtgcagaagcaccagcagcggctgcaggaacagcggcagcagcagagtggggaggcagaagcccttgccagggtgtacagcagcagcattagcaacggcctgagcaacctgaacaacgagaccagcggcacttatgccaacgggcacgtcattgacctgcccaagtctgagggttattacaacgtcgattccggtcagccgtcccctgatcagtcaggacttgacatgactggaatcaaacagataaagcaagaacctatctatgacctcacatccgtacccaacttgtttacctatagctctttcaacaatgggcagttagcaccagggataaccatgactgaaatcgaccgaattgcacagaacatcattaagtcccatttggagacatgtcaatacaccatggaagagctgcaccagctggcgtggcagacccacacctatgaagaaattaaagcatatcaaagcaagtccagggaagcactgtggcaacaatgtgccatccagatcactcacgccatccaatacgtggtggagtttgcaaagcggataacaggcttcatggagctctgtcaaaatgatcaaattctacttctgaagtcaggttgcttggaagtggttttagtgagaatgtgccgtgccttcaacccattaaacaacactgttctgtttgaaggaaaatatggaggaatgcaaatgttcaaagccttaggttctgatgacctagtgaatgaagcatttgactttgcaaagaatttgtgttccttgcagctgaccgaggaggagatcgctttgttctcatctgctgttctgatatctccagaccgagcctggcttatagaaccaaggaaagtccagaagcttcaggaaaaaatttattttgcacttcaacatgtgattcagaagaatcacctggatgatgagaccttggcaaagttaatagccaagataccaaccatcacggcagtttgcaacttgcacggggagaagctgcaggtatttaagcaatctcatccagagatagtgaatacactgtttcctccgttatacaaggagctctttaatcctgactgtgccaccggctgcaaatga

SEQ ID NO: 14 - DNA, 1557 bp, Human
atggacagggccccacagagacagcaccgagcctcacgggagctgctggctgcaaagaagacccacacctcacaaattgaagtgatcccttgcaaaatctgtggggacaagtcgtctgggatccactacggggttatcacctgtgaggggtgcaagggcttcttccgccggagccagcgctgtaacgcggcctactcctgcacccgtcagcagaactgccccatcgaccgcaccagccgaaaccgatgccagcactgccgcctgcagaaatgcctggcgctgggcatgtcccgagatgctgtcaagttcggccgcatgtccaagaagcagagggacagcctgcatgcagaagtgcagaaacagctgcagcagcggcaacagcagcaacaggaaccagtggtcaagacccctccagcaggggcccaaggagcagataccctcacctacaccttggggctcccagacgggcagctgcccctgggctcctcgcctgacctgcctgaggcttctgcctgtccccctggcctcctgaaagcctcaggctctgggccctcatattccaacaacttggccaaggcagggctcaatggggcctcatgccaccttgaatacagccctgagcggggcaaggctgagggcagagagagcttctatagcacaggcagccagctgacccctgaccgatgtggacttcgttttgaggaacacaggcatcctgggcttggggaactgggacagggcccagacagctacggcagccccagtttccgcagcacaccggaggcaccctatgcctccctgacagagatagagcacctggtgcagagcgtctgcaagtcctacagggagacatgccagctgcggctggaggacctgctgcggcagcgctccaacatcttctcccgggaggaagtgactggctaccagaggaagtccatgtgggagatgtgggaacggtgtgcccaccacctcaccgaggccattcagtacgtggtggagttcgccaagaggctctcaggctttatggagctctgccagaatgaccagattgtgcttctcaaagcaggagcaatggaagtggtgctggttaggatgtgccgggcctacaatgctgacaaccgcacggtcttttttgaaggcaaatacggtggcatggagctgttccgagccttgggctgcagcgagctcatcagctccatctttgacttctcccactccctaagtgccttgcacttttccgaggatgagattgccctctacacagcccttgttctcatcaatgcccatcggccagggctccaagagaaaaggaaagtagaacagctgcagtacaatctggagctggcctttcatcatcatctctgcaagactcatcgccaaagcatcctggcaaagctgccacccaaggggaagcttcggagcctgtgtagccagcatgtggaaaggctgcagatcttccagcacctccaccccatcgtggtccaagccgctttccctccactctacaaggagctcttcagcactgaaaccgagtcacctgtggggctgtccaagtga

SEQ ID NO: 15 - DNA, 1443 bp, Human
atgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctga

SEQ ID NO: 16 - DNA, 963 bp, Human
atggagctactgtcgccaccgctccgcgacgtagacctgacggcccccgacggctctctctgctcctttgccacaacggacgacttctatgacgacccgtgtttcgactccccggacctgcgcttcttcgaagacctggacccgcgcctgatgcacgtgggcgcgctcctgaaacccgaagagcactcgcacttccccgcggcggtgcacccggccccgggcgcacgtgaggacgagcatgtgcgcgcgcccagcgggcaccaccaggcgggccgctgcctactgtgggcctgcaaggcgtgcaagcgcaagaccaccaacgccgaccgccgcaaggccgccaccatgcgcgagcggcgccgcctgagcaaagtaaatgaggcctttgagacactcaagcgctgcacgtcgagcaatccaaaccagcggttgcccaaggtggagatcctgcgcaacgccatccgctatatcgagggcctgcaggctctgctgcgcgaccaggacgccgcgccccctggcgccgcagccgccttctatgcgccgggcccgctgcccccgggccgcggcggcgagcactacagcggcgactccgacgcgtccagcccgcgctccaactgctccgacggcatgatggactacagcggccccccgagcggcgcccggcggcggaactgctacgaaggcgcctactacaacgaggcgcccagcgaacccaggcccgggaagagtgcggcggtgtcgagcctagactgcctgtccagcatcgtggagcgcatctccaccgagagccctgcggcgcccgccctcctgctggcggacgtgccttctgagtcgcctccgcgcaggcaagaggctgccgcccccagcgagggagagagcagcggcgaccccacccagtcaccggacgccgccccgcagtgccctgcgggtgcgaaccccaacccgatataccaggtgctctga

SEQ ID NO: 17 - DNA, 3921 bp, Human
atgggggccgcctcgggccgccgggggccggggctgctgctgccgctgccgctgctgttgctgctgccgccgcagcccgccctggcgttggaccccgggctgcagcccggcaacttttctgctgacgaggccggggcgcagctcttcgcgcagagctacaactccagcgccgaacaggtgctgttccagagcgtggccgccagctgggcgcacgacaccaacatcaccgcggagaatgcaaggcgccaggaggaagcagccctgctcagccaggagtttgcggaggcctggggccagaaggccaaggagctgtatgaaccgatctggcagaacttcacggacccgcagctgcgcaggatcatcggagctgtgcgcaccctgggctctgccaacctgcccctggctaagcggcagcagtacaacgccctgctaagcaacatgagcaggatctactccaccgccaaggtctgcctccccaacaagactgccacctgctggtccctggacccagatctcaccaacatcctggcttcctcgcgaagctacgccatgctcctgtttgcctgggagggctggcacaacgctgcgggcatcccgctgaaaccgctgtacgaggatttcactgccctcagcaatgaagcctacaagcaggacggcttcacagacacgggggcctactggcgctcctggtacaactcccccaccttcgaggacgatctggaacacctctaccaacagctagagcccctctacctgaacctccatgccttcgtccgccgcgcactgcatcgccgatacggagacagatacatcaacctcaggggacccatccctgctcatctgctgggagacatgtgggcccagagctgggaaaacatctacgacatggtggtgcctttcccagacaagcccaacctcgatgtcaccagtactatgctgcagcagggctggaacgccacgcacatgttccgggtggcagaggagttcttcacctccctggagctctcccccatgcctcccgagttctgggaagggtcgatgctggagaagccggccgacgggcgggaagtggtgtgccacgcctcggcttgggacttctacaacaggaaagacttcaggatcaagcagtgcacacgggtcacgatggaccagctctccacagtgcaccatgagatgggccatatacagtactacctgcagtacaaggatctgcccgtctccctgcgtcggggggccaaccccggcttccatgaggccattggggacgtgctggcgctctcggtctccactcctgaacatctgcacaaaatcggcctgctggaccgtgtcaccaatgacacggaaagtgacatcaattacttgctaaaaatggcactggaaaaaattgccttcctgccctttggctacttggtggaccagtggcgctggggggtctttagtgggcgtacccccccttcccgctacaacttcgactggtggtatcttcgaaccaagtatcaggggatctgtcctcctgttacccgaaacgaaacccactttgatgctggagctaagtttcatgttccaaatgtgacaccatacatcaggtactttgtgagttttgtcctgcagttccagttccatgaagccctgtgcaaggaggcaggctatgagggcccactgcaccagtgtgacatctaccggtccaccaaggcaggggccaagctccggaaggtgctgcaggctggctcctccaggccctggcaggaggtgctgaaggacatggtcggcttagatgccctggatgcccagccgctgctcaagtacttccagccagtcacccagtggctgcaggagcagaaccagcagaacggcgaggtcctgggctggcccgagtaccagtggcacccgccgttgcctgacaactacccggagggcatagacctggtgactgatgaggctgaggccagcaagtttgtggaggaatatgaccggacatcccaggtggtgtggaacgagtatgccgaggccaactggaactacaacaccaacatcaccacagagaccagcaagattctgctgcagaagaacatgcaaatagccaaccacaccctgaagtacggcacccaggccaggaagtttgatgtgaaccagttgcagaacaccactatcaagcggatcataaagaaggttcaggacctagaacgggcagcactgcctgcccaggagctggaggagtacaacaagatcctgttggatatggaaaccacctacagcgtggccactgtgtgccacccgaatggcagctgcctgcagctcgagccagatctgacgaatgtgatggccacgtcccggaaatatgaagacctgttatgggcatgggagggctggcgagacaaggcggggagagccatcctccagttttacccgaaatacgtggaactcatcaaccaggctgcccggctcaatggctatgtagatgcaggggactcgtggaggtctatgtacgagacaccatccctggagcaagacctggagcggctcttccaggagctgcagccactctacctcaacctgcatgcctacgtgcgccgggccctgcaccgtcactacggggcccagcacatcaacctggaggggcccattcctgctcacctgctggggaacatgtgggcgcagacctggtccaacatctatgacttggtggtgcccttcccttcagccccctcgatggacaccacagaggctatgctaaagcagggctggacgcccaggaggatgtttaaggaggctgatgatttcttcacctccctggggctgctgcccgtgcctcctgagttctggaacaagtcgatgctggagaagccaaccgacgggcgggaggtggtctgccacgcctcggcctgggacttctacaacggcaaggacttccggatcaagcagtgcaccaccgtgaacttggaggacctggtggtggcccaccacgaaatgggccacatccagtatttcatgcagtacaaagacttacctgtggccttgagggagggtgccaaccccggcttccatgaggccattggggacgtgctagccctctcagtgtctacgcccaagcacctgcacagtctcaacctgctgagcagtgagggtggcagcgacgagcatgacatcaactttctgatgaagatggcccttgacaagatcgcctttatccccttcagctacctcgtcgatcagtggcgctggagggtatttgatggaagcatcaccaaggagaactataaccaggagtggtggagcctcaggctgaagtaccagggcctctgccccccagtgcccaggactcaaggtgactttgacccaggggccaagttccacattccttctagcgtgccttacatcaggtactttgtcagcttcatcatccagttccagttccacgaggcactgtgccaggcagctggccacacgggccccctgcacaagtgtgacatctaccagtccaaggaggccgggcagcgcctggcgaccgccatgaagctgggcttcagtaggccgtggccggaagccatgcagctgatcacgggccagcccaacatgagcgcctcggccatgttgagctacttcaagccgctgctggactggctccgcacggagaacgagctgcatggggagaagctgggctggccgcagtacaactggacgccgaactccgctcgctcagaagggcccctcccagacagcggccgcgtcagcttcctgggcctggacctggatgcgcagcaggcccgcgtgggccagtggctgctgctcttcctgggcatcgccctgctggtagccaccctgggcctcagccagcggctcttcagcatccgccaccgcagcctccaccggcactcccacgggccccagttcggctccgaggtggagctgagacactcctga

SEQ ID NO: 18 - DNA, 2397 bp, Human
atggcgtgggacatgtgcaaccaggactctgagtctgtatggagtgacatcgagtgtgctgctctggttggtgaagaccagcctctttgcccagatcttcctgaacttgatctttctgaactagatgtgaacgacttggatacagacagctttctgggtggactcaagtggtgcagtgaccaatcagaaataatatccaatcagtacaacaatgagccttcaaacatatttgagaagatagatgaagagaatgaggcaaacttgctagcagtcctcacagagacactagacagtctccctgtggatgaagacggattgccctcatttgatgcgctgacagatggagacgtgaccactgacaatgaggctagtccttcctccatgcctgacggcacccctccaccccaggaggcagaagagccgtctctacttaagaagctcttactggcaccagccaacactcagctaagttataatgaatgcagtggtctcagtacccagaaccatgcaaatcacaatcacaggatcagaacaaaccctgcaattgttaagactgagaattcatggagcaataaagcgaagagtatttgtcaacagcaaaagccacaaagacgtccctgctcggagcttctcaaatatctgaccacaaacgatgaccctcctcacaccaaacccacagagaacagaaacagcagcagagacaaatgcacctccaaaaagaagtcccacacacagtcgcagtcacaacacttacaagccaaaccaacaactttatctcttcctctgaccccagagtcaccaaatgaccccaagggttccccatttgagaacaagactattgaacgcaccttaagtgtggaactctctggaactgcaggcctaactccacccaccactcctcctcataaagccaaccaagataacccttttagggcttctccaaagctgaagtcctcttgcaagactgtggtgccaccaccatcaaagaagcccaggtacagtgagtcttctggtacacaaggcaataactccaccaagaaagggccggagcaatccgagttgtatgcacaactcagcaagtcctcagtcctcactggtggacacgaggaaaggaagaccaagcggcccagtctgcggctgtttggtgaccatgactattgccagtcaattaattccaaaacagaaatactcattaatatatcacaggagctccaagactctagacaactagaaaataaagatgtctcctctgattggcaggggcagatttgttcttccacagattcagaccagtgctacctgagagagactttggaggcaagcaagcaggtctctccttgcagcacaagaaaacagctccaagaccaggaaatccgagccgagctgaacaagcacttcggtcatcccagtcaagctgtttttgacgacgaagcagacaagaccggtgaactgagggacagtgatttcagtaatgaacaattctccaaactacctatgtttataaattcaggactagccatggatggcctgtttgatgacagcgaagatgaaagtgataaactgagctacccttgggatggcacgcaatcctattcattgttcaatgtgtctccttcttgttcttcttttaactctccatgtagagattctgtgtcaccacccaaatccttattttctcaaagaccccaaaggatgcgctctcgttcaaggtccttttctcgacacaggtcgtgttcccgatcaccatattccaggtcaagatcaaggtctccaggcagtagatcctcttcaagatcctgctattactatgagtcaagccactacagacaccgcacgcaccgaaattctcccttgtatgtgagatcacgttcaagatcgccctacagccgtcggcccaggtatgacagctacgaggaatatcagcacgagaggctgaagagggaagaatatcgcagagagtatgagaagcgagagtctgagagggccaagcaaagggagaggcagaggcagaaggcaattgaagagcgccgtgtgatttatgtcggtaaaatcagacctgacacaacacggacagaactgagggaccgttttgaagtttttggtgaaattgaggagtgcacagtaaatctgcgggatgatggagacagctatggtttcattacctaccgttatacctgtgatgcttttgctgctcttgaaaatggatacactttgcgcaggtcaaacgaaactgactttgagctgtacttttgtggacgcaagcaatttttcaagtctaactatgcagacctagattcaaactcagatgactttgaccctgcttccaccaagagcaagtatgactctctggattttgatagtttactgaaagaagctcagagaagcttgcgcaggtaa

SEQ ID NO: 19 - DNA, 900 bp, Human
atggaacattttgatgcatcacttagtacctatttcaaggcattgctaggccctcgagatactagagtaaaaggatggtttcttctggacaattatatacccacatttatctgctctgtcatatatttactaattgtatggctgggaccaaaatacatgaggaataaacagccattctcttgccgggggattttagtggtgtataaccttggactcacactgctgtctctgtatatgttctgtgagttagtaacaggagtatgggaaggcaaatacaacttcttctgtcagggcacacgcaccgcaggagaatcagatatgaagattatccgtgtcctctggtggtactacttctccaaactcatagaatttatggacactttcttcttcatcctgcgcaagaacaaccaccagatcacggtcctgcacgtctaccaccatgcctcgatgctgaacatctggtggtttgtgatgaactgggtcccctgcggccactcttattttggtgccacacttaatagcttcatccacgtcctcatgtactcttactatggtttgtcgtcagtcccttccatgcgtccatacctctggtggaagaagtacatcactcaggggcagctgcttcagtttgtgctgacaatcatccagaccagctgcggggtcatctggccgtgcacattccctcttggttggttgtatttccagattggatacatgatttccctgattgctctcttcacaaacttctacattcagacctacaacaagaaaggggcctcccgaaggaaagaccacctgaaggaccaccagaatgggtccatggctgctgtgaatggacacaccaacagcttttcacccctggaaaacaatgtgaagccaaggaagctgcggaaggattga

SEQ ID NO: 20 - DNA, 1530 bp, Human
atgccgtcgggcttccaacagataggctccgaagatggggaaccccctcagcagcgagtgactgggaccctggtccttgctgtgttctctgcggtgcttggctccctgcagtttgggtacaacattggggtcatcaatgcccctcagaaggtgattgaacagagctacaatgagacgtggctggggaggcaggggcctgagggacccagctccatccctccaggcaccctcaccaccctctgggccctctccgtggccatcttttccgtgggcggcatgatttcctccttcctcattggtatcatctctcagtggcttggaaggaaaagggccatgctggtcaacaatgtcctggcggtgctggggggcagcctcatgggcctggccaatgctgctgcctcctatgaaatgctcatccttggacgattcctcattggcgcctactcagggctgacatcagggctggtgcccatgtacgtgggggagattgctcccactcacctgcggggcgccctggggacgctcaaccaactggccattgttatcggcattctgatcgcccaggtgctgggcttggagtccctcctgggcactgccagcctgtggccactgctcctgggcctcacagtgctacctgccctcctgcagctggtcctgctgcccttctgtcccgagagcccccgctacctctacatcatccagaatctcgaggggcctgccagaaagagtctgaagcgcctgacaggctgggccgatgtttctggagtgctggctgagctgaaggatgagaagcggaagctggagcgtgagcggccactgtccctgctccagctcctgggcagccgtacccaccggcagcccctgatcattgcggtcgtgctgcagctgagccagcagctctctggcatcaatgctgttttctattattcgaccagcatcttcgagacagcaggggtaggccagcctgcctatgccaccataggagctggtgtggtcaacacagtcttcaccttggtctcggtgttgttggtggagcgggcggggcgccggacgctccatctcctgggcctggcgggcatgtgtggctgtgccatcctgatgactgtggctctgctcctgctggagcgagttccagccatgagctacgtctccattgtggccatctttggcttcgtggcattttttgagattggccctggccccattccttggttcatcgtggccgagctcttcagccagggaccccgcccggcagccatggctgtggctggtttctccaactggacgagcaacttcatcattggcatgggtttccagtatgttgcggaggctatggggccctacgtcttccttctatttgcggtcctcctgctgggcttcttcatcttcaccttcttaagagtacctgaaactcgaggccggacgtttgaccagatctcagctgccttccaccggacaccctctcttttagagcaggaggtgaaacccagcacagaacttgagtatttagggccagatgagaacgactga